(19) United States
(12) Patent Application Publication
(10) Pub. No.: US 2022/0257435 A1
(43) Pub. Date: Aug. 18, 2022

(54) COMPOSITE HAVING IMPROVED IN-PLANE PERMEABILITY AND ABSORBENT ARTICLE HAVING IMPROVED FLUID MANAGEMENT

(71) Applicant: INTERNATIONAL PAPER COMPANY, Memphis, TN (US)

(72) Inventors: CHARLES E. MILLER, FEDERAL WAY, WA (US); RYAN JOEL ENG, SEATTLE, WA (US); JUN ZHANG, BELLEVUE, WA (US); JANE MARICEL DIEGO-DEGUZMAN, LAKEWOOD, WA (US)

(21) Appl. No.: 17/678,588

(22) Filed: Feb. 23, 2022

Related U.S. Application Data
(63) Continuation-in-part of application No. 17/674,965, filed on Feb. 18, 2022.
Application No. 17/674,965 is a continuation of application No. PCT/US2021/047342, filed on Aug. 24, 2021.
(60) Provisional application No. 63/158,471, filed on Mar. 9, 2021.
(60) Provisional application No. 63/069,678, filed on Aug. 24, 2020.
(60) Provisional application No. 63/158,471, filed on Mar. 9, 2021.

(51) Int. Cl. A61F 13/531; A61L 15/22; A61F 13/515

(52) U.S. Cl. CPC A61F 13/531; A61F 13/472; A61F 13/515; A61L 15/225

(57) ABSTRACT

The present disclosure features a feminine hygiene product, including a nonwoven layer including polymeric fibers and/or filaments; a crosslinked cellulose layer including crosslinked cellulose fibers; wherein the crosslinked cellulose layer is positioned opposed to the nonwoven layer (e.g., without an intervening layer different from the crosslinked cellulose layer and the nonwoven layer; in some embodiments, the crosslinked cellulose layer is immediately adjacent to the nonwoven layer); and an interfacial region between the nonwoven layer and the crosslinked cellulose layer, the interfacial region including physically entangled polymeric fibers and/or filaments from the nonwoven layer and crosslinked cellulose fibers from the crosslinked cellulose layer. The nonwoven layer and the crosslinked cellulose layer of the composite fabric are mechanically inseparable in a dry state.

### CROSS-REFERENCE TO RELATED APPLICATION

This application claims the benefit of U.S. Patent Application No. 63/158,471, filed Mar. 9, 2021 and U.S. patent application Ser. No. 17/674,965, filed Feb. 18, 2022, the disclosure of each of which is incorporated herein by reference in its entirety.

### BACKGROUND

Personal care absorbent products, such as baby diapers, adult incontinent pads and undergarments, and feminine care products, typically contain a fluid absorbent core. Many absorbent articles include the fluid absorbent core disposed between a topsheet and a backsheet. The topsheet is typically formed from a fluid-permeable material adapted to promote fluid transfer into the absorbent core, such as upon a liquid insult, usually with minimal fluid retention by the topsheet. U.S. southern pine fluff pulp is commonly used in the absorbent core, generally in the form of a fibrous matrix, and sometimes in conjunction with a superabsorbent polymer (SAP) dispersed throughout the fibrous matrix. This fluff pulp is recognized worldwide as the preferred fiber for absorbent products, based on factors such as the fluff pulp's high fiber length, fiber coarseness, and its relative ease of processing from a wet-laid and dried pulp sheet to an air-laid web. The raw material for this type of cellulosic fluff pulp is Southern Pine (e.g., Loblolly Pine, Pinus taeda L.). The raw material is renewable, and the pulp is easily biodegradable. Compared to SAP, these fibers are inexpensive on a per mass basis but tend to be more expensive on per unit of liquid held basis. These fluff pulp fibers mostly absorb within the interstices between fibers.

SAPs are water-swellable, generally water-insoluble absorbent materials having a high absorbent capacity for fluids. SAP, upon absorption of fluids, swells and becomes a gel holding more than its weight of such fluids. The SAPs in common use are mostly derived from acrylic acid. Acrylic acid based polymers also include a meaningful portion of the cost structure of diapers, incontinent pads and undergarment. SAPs are designed to have high gel strength (as demonstrated by high absorbency under load or AUL). The high gel strength (upon swelling) of currently used SAP particles helps them to retain significant void space between particles, which is helpful for rapid fluid uptake. However, this high “void volume” simultaneously results in significant interstitial (between particles) liquid in the product in the saturated state.

While fluff pulp fibers and SAP can store very large amounts of liquid, they are often not able to distribute the liquid from the point of insult to more remote areas of the absorbent article and to acquire the liquid as fast as it may be received by the article. For this reason, acquisition members are used, which provide for the interim acquisition of large amounts of liquid and which often also allow for the distribution of liquid. Thereby the acquisition member plays an important role in using the whole absorbent capacity provided by the storage member.

Materials suitable to meet the above outlined requirements for a liquid acquisition layer must meet these requirements not only in standard or ideal conditions, but in a variety of conditions, namely at different temperatures and pressures, occurring in use, but also during storage and handling.

Some absorbent articles, such as diapers or adult incontinence pads, include an acquisition and distribution layer (ADL) for the collection and uniform and timely distribution of fluid from a fluid insult to the absorbent core. An ADL is usually placed between the topsheet and the absorbent core, and can, for example, take the form of composite fabric with the top-one third of the fabric having higher denier fiber with relatively large voids and higher void volume for the effective acquisition of the presented fluid, even at relatively higher discharge rates. The middle one-third of the composite fabric of the ADL can be made of low denier fibers with smaller voids, while the lower one-third of the fabric can be made of even lower and smaller denier fibers and yet with finer voids. The higher density portions of the composite have more and finer capillaries and hence develop greater capillary pressure, thus moving greater volumes of fluid to the outer regions of the structure thus enabling the proper channelization and distribution of the fluid in an even fashion to allow the absorbent core to take up all of the liquid insult in a time bound manner to allow SAP within the absorbent core to hold and to gel the insult neither too slow nor too fast. The ADL provides for more rapid liquid acquisition (minimizing flooding in the target zone) and ensures more rapid transport and thorough distribution of the fluid into the absorbent core.

There is a need for a fluid distribution layer or a core-wrap material having improved liquid handling characteristics as compared to the above-disclosed articles. There is a need for an absorbent article, which is more comfortable to wear, and which in particular provides superior dryness. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a composite fabric, including: a nonwoven layer including polymeric fibers and/or filaments; a crosslinked cellulose layer including crosslinked cellulose fibers; wherein the crosslinked cellulose layer is positioned opposed to the nonwoven layer (e.g., without an intervening layer different from the crosslinked cellulose layer and the nonwoven layer; in some embodiments, the crosslinked cellulose layer is immediately adjacent to the nonwoven layer); and an interfacial region between the nonwoven layer and the crosslinked cellulose layer, including physically entangled polymeric fibers and/or filaments from the nonwoven layer and crosslinked cellulose fibers from the crosslinked cellulose layer, wherein the nonwoven layer and the crosslinked cellulose layer are mechanically inseparable in a dry state, and wherein the composite fabric has a density of from 0.06 g/cm³ to 0.15 g/cm³ (e.g., 0.06 g/cm³, 0.12 g/cm³, 0.08 g/cm³, or 0.06-0.08 g/cm³).

In another aspect, the present disclosure features an absorbent article, including the composite fabric described herein.

In yet another aspect, the present disclosure features an absorbent article, including: a liquid-impermeable backsheet defining an inner surface and an outer surface; an absorbent core, disposed on the inner surface of the backsheet, and a topsheet overlying the upper surface of the absorbent core and contacting the inner surface of the backsheet. The absorbent core includes: an absorbent material defining an upper surface and a lower surface of the absorbent core; and a composite fabric surrounding at least a portion of the upper surface and the lower surface, including: a nonwoven layer including polymeric fibers and/or filaments; a crosslinked cellulose layer including crosslinked cellulose fibers, wherein the crosslinked cellulose layer is positioned opposed to the nonwoven layer; and an interfacial region between the nonwoven layer and the crosslinked cellulose layer, including physically entangled polymeric fibers and/or filaments from the nonwoven layer and crosslinked cellulose fibers from the crosslinked cellulose layer, wherein the nonwoven layer and the crosslinked cellulose layer are mechanically inseparable in a dry state.

In yet a further aspect, the present disclosure features a feminine hygiene product, including: a composite fabric including a nonwoven layer including polymeric fibers and/or filaments; a crosslinked cellulose layer including crosslinked cellulose fibers, wherein the crosslinked cellulose layer is positioned opposed to the nonwoven layer; and an interfacial region between the nonwoven layer and the crosslinked cellulose layer, including physically entangled polymeric fibers and/or filaments from the nonwoven layer and crosslinked cellulose fibers from the crosslinked cellulose layer, wherein the nonwoven layer and the crosslinked cellulose layer are mechanically inseparable in a dry state.

In yet a further aspect, the present disclosure features a method of making a composite fabric of the present disclosure, including supplying polymeric fibers and/or filaments; supplying crosslinked cellulose fibers; air-laying or wet-laying the crosslinked cellulose fibers to provide a crosslinked cellulose layer on a nonwoven layer of polymeric fibers and/or filaments, wherein the crosslinked cellulose layer is positioned opposed to the nonwoven layer; and physically entangling the polymeric fibers and/or filaments from the nonwoven layer and the crosslinked cellulose fibers from the crosslinked cellulose layer to provide the composite fabric, wherein the composite fabric includes an interfacial region between the nonwoven layer and the crosslinked cellulose layer, wherein the nonwoven layer and the crosslinked cellulose layer are mechanically inseparable in a dry state.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a schematic representation of a hydro-entangling process of the present disclosure.

FIG. 2A is a schematic representation of an embodiment of a fluid acquisition and distribution layer (ADL) of the present disclosure.

FIG. 2B is schematic cross-sectional representation of an embodiment of a core-wrap of the present disclosure.

FIG. 3 is a schematic cross-sectional representation of an embodiment of a core-wrap of the present disclosure.

FIG. 4 is a schematic cross-sectional representation of an embodiment of an absorbent article of the present disclosure.

FIG. 5A is a schematic cross-sectional representation of an embodiment of an absorbent article of the present disclosure.

FIG. 5B is a schematic cross-sectional representation of an embodiment of an absorbent article of the present disclosure.

FIG. 5C is a schematic cross-sectional representation of an embodiment of an absorbent article of the present disclosure.

FIG. 5D is a schematic cross-sectional representation of an embodiment of an absorbent article of the present disclosure.

FIG. 5E is a schematic cross-sectional representation of an embodiment of an absorbent article of the present disclosure.

FIG. 6A is a schematic cross-sectional representation of an embodiment of an absorbent article of the present disclosure.

FIG. 6B is a schematic cross-sectional representation of an embodiment of an absorbent article of the present disclosure.

FIG. 6C is a schematic cross-sectional representation of an embodiment of an absorbent article of the present disclosure.

FIG. 6D is a schematic cross-sectional representation of an embodiment of an absorbent article of the present disclosure.

FIG. 7A is a schematic cross-sectional representation of an embodiment of an absorbent article of the present disclosure.

FIG. 7B is a schematic cross-sectional representation of an embodiment of an absorbent article of the present disclosure.

FIG. 8A is a schematic cross-sectional representation of an embodiment of an absorbent article of the present disclosure.

FIG. 8B is a schematic cross-sectional representation of an embodiment of an absorbent article of the present disclosure.

FIG. 8C is a schematic cross-sectional representation of an embodiment of an absorbent article of the present disclosure.

FIG. 9 is a bar graph showing a comparison of wicking distance from insult point of an embodiment of an ADL diaper construct of the present disclosure vs. a commercial diaper in a no load saddle wicking test.

FIG. 10 is a bar graph showing a comparison of intake times of an embodiment of an ADL diaper construct of the present disclosure vs. a commercial diaper in a flat acquisition under load test.

FIG. 11 is a bar graph showing a comparison of rewet values of an embodiment of an ADL diaper construct of the present disclosure vs. a commercial diaper in a flat acquisition under load test.

FIG. 12 is a bar graph showing a comparison of wicking distances of an embodiment of an ADL diaper constructs of the present disclosure vs. a commercial diaper in a flat acquisition under load test.

FIG. 13 is a bar graph showing a comparison of intake times of embodiments of a core-wrap diaper construct of the present disclosure vs. a commercial diaper in a no load saddle wicking test.

FIG. 14 is a bar graph showing a comparison of wicking distances from insult point of an embodiment of a core-wrap diaper construct of the present disclosure vs. a commercial diaper in a no load saddle wicking test.

FIG. 15 is a bar graph showing a comparison of intake times of an embodiment of a core-wrap diaper construct of the present disclosure vs. a commercial diaper in a flat acquisition under load test.

FIG. 16 is a bar graph showing a comparison of rewet values of an embodiment of a core-wrap diaper construct of the present disclosure vs. a commercial diaper in a flat acquisition under load test.

FIG. 17 is a bar graph showing a comparison of wicking distances of an embodiment of a core-wrap diaper construct of the present disclosure vs. a commercial diaper in a flat acquisition under load test.

FIG. 18 is a bar graph showing intake times of an embodiment of a core-wrap diaper construct of the present disclosure vs. an average of commercial fluffless diapers in a no load saddle wicking test.

FIG. 19 is a bar graph showing a comparison of wicking distance from insult point of an embodiment of a core-wrap diaper construct of the present disclosure vs. an average of commercial fluffless diapers in a no load saddle wicking test.

FIG. 20 is a bar graph showing intake times of an embodiment of a core-wrap diaper construct of the present disclosure vs. an average of commercial fluffless diapers in a flat acquisition under load test.

FIG. 21 is a bar graph showing a comparison of rewet values of an embodiment of a core-wrap diaper construct of the present disclosure vs. an average of commercial fluffless diapers in a flat acquisition under load test.

FIG. 22 is a bar graph showing a comparison of wicking distances of an embodiment of a core-wrap diaper construct of the present disclosure vs. an average of commercial fluffless diapers in a flat acquisition under load test.

FIG. 23 is a bar graph showing a comparison of wicking distances from insult point of an embodiment of an ADL diaper construct of the present disclosure vs. an average of commercial fluff core diapers in a no load saddle wicking test.

FIG. 24 is a bar graph showing a comparison of wicking distances from insult point of an embodiment of an ADL diaper construct of the present disclosure vs. an average of commercial fluff core diapers in a flat acquisition under load test.

FIG. 25 is a photograph of an embodiment of a feminine hygiene absorbent core of the present disclosure following a rewet and distribution test.

FIG. 26A is a schematic diagram illustrating an example feminine hygiene product 2600A including an absorbent composite 2610A, in accordance with embodiments of the present disclosure.

FIG. 26B is a schematic diagram illustrating a multilayer configuration where an absorbent composite includes one or more stacked layers of the physically entangled composite fabric described in reference to FIG. 1, in accordance with embodiments of the present disclosure.

FIG. 27A illustrates a comparative example feminine hygiene product 2700 as available in the North American market.

FIG. 27B illustrates a comparative example feminine hygiene product 2750 as available in the East Asian market.

FIG. 28A is a schematic diagram of a cross section of example feminine hygiene product of FIG. 26A illustrating material layers of absorbent composite material, in accordance with embodiments of the present disclosure.

FIG. 28B is a schematic diagram illustrating a cross section of example feminine hygiene product of FIG. 26B, including absorbent composite material formed of multiple stacked layers of composite formed of crosslinked cellulose layer physically entangled with nonwoven layer, with interfacial region formed therebetween, in accordance with embodiments of the present disclosure.

FIG. 29 is a graph of intake time data demonstrating improvement of a shared property between a negative control product without cellulosic materials, comparative example 1, comparative example 2, and example 1 and example 2 in accordance with embodiments of the present disclosure.

FIG. 30 is a graph of performance assay data of intake time for comparative example 1 and example 1, highlighting improved intake time performance of example feminine hygiene products relative to comparative products from the North American market, in accordance with embodiments of the present disclosure.

FIG. 31 is a graph of data from rewet mass assays for the same set of five products. As previously described, rewet mass measures a quantity of liquid that is exuded from an absorbent article following application of a set pressure, in accordance with embodiments of the present disclosure.

FIG. 32 is a graph of data from wicking distance assays for the same set of five products. As previously described, wicking distance measures the performance of feminine hygiene products to accept and distribute a liquid insult during a prescribed period of time, in accordance with embodiments of the present disclosure.

### DETAILED DESCRIPTION

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It is appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below” and words of similar import, when used in this application, shall refer to this application as a whole and not to any particular portions of the application.

As used herein, “absorbent article” refers to products that absorb and contain liquid, and more specifically, refers to products that are placed against or in proximity to the body of the wearer to absorb and contain the various exudates discharged from the body. Absorbent articles include but are not limited to diapers, adult incontinent briefs, training pants, diaper holders and liners, sanitary napkins and the like. These articles can include a topsheet, a backsheet, an absorbent core, and optionally a receiving layer and/or a distribution layer, and other components, wherein the absorbent core is normally disposed between the backsheet and the receiving system or the topsheet. Absorbent articles also include wipes, such as household cleaning wipes, baby wipes, and the like.

As used herein, the term “absorbent core” refers to a single component that is disposed or disposed in an absorbent article and that includes an absorbent material encased in a core-wrap.

The core-wrap can be a sheet that envelops the absorbent material and can, for example, include the composite fabric of the present disclosure. The term “absorbent core” does not extend to a receiving or distribution layer or any other component of an absorbent article that is not an integral part of the core-wrap or that is not disposed within the core-wrap. The absorbent core can have the highest absorbency in the absorbent article and can include superabsorbent polymers (SAP) and/or fluff pulp.

As used herein, the term “disposable” refers to articles that are generally not intended to be laundered or otherwise restored or reused, i.e., they are intended to be discarded after a single use and, possibly, to be recycled, composted or otherwise disposed of in an environmentally compatible manner.

As used herein, the term “disposed” refers to an element(s) that is formed (joined and positioned) in a particular place or position as a unitary structure with other elements or as a separate element joined to another element.

As used herein, the term “diaper” refers to an absorbent article generally worn by infants and incontinent persons about the lower torso.

The terms “thickness” and “caliper” are used herein interchangeably.

As used herein, the terms “nonwoven,” “nonwoven fabric,” and “nonwoven web” are interchangeable and refer to a sheet, web or mat product made of directionally or randomly disposed fibers and/or filaments bonded together by friction and/or by cohesion and/or adhesion. The fibers can be of natural (e.g., cotton) or regenerated (e.g., regenerated cellulose) or synthetic origin and can be staple or continuous fibers or formed in situ. The fibers can have diameters ranging from less than about 0.001 mm to more than 0.2 mm, and can be available in several different forms, for example, as short fibers (so-called staple or cut fibers), continuous single fibers (filaments or monofilaments), untwisted bundles of continuous filaments (cables) and twisted bundles of continuous fibers (yarn). Nonwoven webs can be formed by various processes, such as meltblowing, spunbonding, solvent spinning, electrospinning, carding and aerodynamic laying or air-laying, or any combination thereof. The basis weight of nonwoven webs is usually expressed in grams per square meter (g/m², G, or gsm), respectively. Synthetic fibers and/or filaments include but are not limited to polyolefins such as polypropylene, polyethylene, and polyester (e.g., polyethylene terephthalate), and any combination thereof (e.g., a bicomponent fiber).

As used herein, Helix™ is a crosslinked cellulose fiber based on untreated fluff pulp (such as SuperSoft® from International Paper Company). Methods of manufacturing Helix™ are described, for example, in U.S. Pat. Nos. 5,399,240, 5,437,418, and 6,436,231, each of which is herein incorporated by reference in its entirety.

As used herein, Helix™ Air®+ is a crosslinked fiber based on a treated or debonded fluff grade (such as SuperSoft® Air® and/or SuperSoft® Air®+). Methods of manufacturing Helix™ are described, for example, in U.S. Pat. Nos. 5,399,240, 5,437,418, and 6,436,231, each of which is herein incorporated by reference in its entirety. Debonded pulp is described, for example, in U.S. Pat. No. 6,306,251, herein incorporated in its entirety.

In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

As used herein, with respect to measurements, “about” means+/−5%. As used herein, a recited range includes the end points, such that from 0.5 mole percent to 99.5 mole percent includes both 0.5 mole percent and 99.5 mole percent.

The principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

### Composite Fabric

Absorbent products are increasingly thin and flexible. Consequently, a loss of void volume in the absorbent core has occurred, which in turn requires more powerful absorbent systems for fluid management to deliver acceptable leakage protection for the consumer.

The present disclosure describes a composite fabric that includes crosslinked cellulose fiber and a nonwoven that can be used in an absorbent article, such as in an acquisition and distribution layer (“ADL”) and/or in a core-wrap of the absorbent article. Crosslinked cellulose fiber has unique properties such as excellent wet bulk and resiliency that are advantageous in absorbent articles. However, commercially available crosslinked cellulose fiber is in a compressed bale format that limits its application in most manufacturing facilities due to the lack of bale openers in many commercial operations. A rolled format of crosslinked cellulose fiber can increase convenience and simplify manufacturing processes. As will be described in more detail below, a web composed of crosslinked cellulose fibers can be formed by an air-laid or wet-laid process, and subsequently entangled into a nonwoven fabric, such as bonded carded web (BCW) to form a composite fabric. The cellulosic fiber penetration into the nonwoven fabric can be controlled (e.g., by controlling water jet pressure in a hydroentangling process), and the composite fabric can have a dual layer structure with little crosslinked cellulose fiber penetration in the nonwoven to a completely interpenetrated network of crosslinked cellulose fiber in the nonwoven.

Thus, the present disclosure features a composite fabric, including a nonwoven layer including polymeric fibers and/or filaments; a crosslinked cellulose layer including crosslinked cellulose fibers; wherein the crosslinked cellulose layer is positioned opposed to the nonwoven layer; and an interfacial region between the nonwoven layer and the crosslinked cellulose layer, the interfacial region including physically entangled polymeric fibers and/or filaments from the nonwoven layer and crosslinked cellulose fibers from the crosslinked cellulose layer. The nonwoven layer and the crosslinked cellulose layer of the composite fabric are mechanically inseparable in a dry state. The composite fabric has a density of from 0.06 g/cm³ to 0.15 g/cm³ (e.g., 0.06 g/cm³, 0.12 g/cm³, 0.08 g/cm³, or 0.06-0.08 g/cm³). The density is measured according to the method “Thickness, Bulk, and Density Measurement” described below. Average density is the average of at least 5 density values measured in a sample. The crosslinked cellulose layer is position opposed to the nonwoven layer without an intervening layer different from the crosslinked cellulose layer and the nonwoven layer. In some embodiments, the crosslinked cellulose layer is immediately adjacent to and entangled in the nonwoven layer. In some embodiments, the composite fabric consists essentially of the nonwoven layer and the crosslinked cellulose layer, and an interfacial region between the nonwoven layer and the crosslinked cellulose layer. In some embodiments, the composite fabric consists of the nonwoven layer and the crosslinked cellulose layer, and an interfacial region between the nonwoven layer and the crosslinked cellulose layer.

In some embodiments, the polymeric fibers and/or filaments of the nonwoven layer include synthetic polymer fibers and/or filaments, such as polyolefin and/or polyester fibers and/or filaments. The nonwoven layer can include webs, which can be produced by a melt spun process.

In some embodiments, the nonwoven layer is a bonded carded web. In some embodiments, the nonwoven layer includes a bonded carded web fabric, a carded web, a spunbond fabric, a melt blown fabric, an unbonded synthetic fiber, or any combination thereof.

In some embodiments, the nonwoven layer and the crosslinked cellulose layer overlap (i.e., overlay one another) and interpenetrate at the interfacial region. In some embodiments, the crosslinked cellulose layer and the nonwoven layer fully interpenetrate.

The composite fabric can have an “x” dimension and a “y” dimension corresponding to the width and length of the composite fabric. The composite fabric can further have a “z” dimension, corresponding to the thickness of the composite fabric. In some embodiments, the nonwoven layer has a first thickness, the crosslinked cellulose layer has a second thickness, and the interfacial region has a thickness that is less than or equal to the thickness of the first or the second thickness. In some embodiments, the interfacial region can have a thickness that spans the entire thickness of the nonwoven layer, when the crosslinked cellulose layer is fully entangled in the nonwoven layer. In some embodiments, the interfacial region can have a thickness that is less than the thickness of the nonwoven layer and/or the crosslinked cellulose layer when the crosslinked cellulose layer is partially entangled in the nonwoven layer.

In some embodiments, the composite fabric has regions where the crosslinked cellulose layer has greater entanglement into the nonwoven layer than other regions, such that the interfacial region can vary in thickness. Without wishing to be bound by theory, it is believed that when the composite fabric has interfacial regions of greater entanglement, pathways or channels can form in the composite fabric to guide the flow of liquids through the composite fabric.

In some embodiments, the nonwoven layer can include a bonded carded web fabric (e.g., a resin bonded carded web fabric), a carded web, a spunbond fabric, a melt directionally or blown fabric, an unbonded synthetic fiber, staple fibers (e.g., synthetic fibers laid down as a mat and not bonded by any mechanism), or any combination thereof. A nonwoven fabric can include a manufactured sheet, web or batt of randomly orientated fibers and/or filaments, bonded by friction, and/or cohesion and/or adhesion, excluding paper and products which are woven, knitted, tufted, stitch-bonded incorporating binding yarns or filaments, or felted by wet-milling, whether or not additionally needled. The fibers and/or filaments in the nonwoven fabric layer can be synthetic or of natural origin, such as polyolefins (e.g., polypropylene, polyethylene), polyesters, or any combination thereof (e.g., a bicomponent fiber).

Commercially available fibers can have diameters ranging from less than about 0.001 mm to more than about 0.2 mm and take the form of short fibers (staple or chopped), continuous single fibers (filaments or monofilaments), untwisted bundles of continuous filaments (tow), and twisted bundles of continuous filaments (yarn). Fibers are classified according to their origin, chemical structure, or both.

Nonwoven webs can be formed by direct extrusion processes during which the fibers and webs are formed at about the same point in time, or by preformed fibers, which can be laid into webs at a distinctly subsequent point in time. Example direct extrusion processes include but are not limited to: spunbonding, meltblowing, solvent spinning, electrospinning, and combinations thereof typically forming layers.

All of the above-described fibers and manufacturing techniques can be useful for providing the composite fabric according to the present disclosure.

The crosslinked cellulose fibers can include polyacrylic acid crosslinked cellulose fibers. Crosslinked cellulose fibers are described, for example, in U.S. Pat. Nos. 7,513,973, 8,722,797, 6,716,306, 6,736,933, 6,748,671, 7,018,508, 6,782,637, 6,865,822; 7,290,353, 6,769,199, 7,147,446, 7,399,377, 6,306,251, 5,183,707, and 5,998,511, each of which is incorporated herein in its entirety. Example crosslinking mechanisms include esterification reactions, etherification, ionic reactions, and radical reactions. As example, the crosslinked cellulose fibers include bleached polyacrylic acid crosslinked cellulosic fibers, where polyacrylic acid crosslinked cellulosic fibers are treated with one or more bleaching agents to provide crosslinked cellulosic fibers having high bulk and improved whiteness. In another example, the crosslinked cellulose fibers can include polyacrylic acid crosslinking agent that includes a polyacrylic acid, having phosphorous incorporated into the polymer chain (as a phosphinate) by introduction of sodium hypophosphite during the polymerization process.

For example, individualized, chemically crosslinked cellulosic fibers can be intrafiber crosslinked with a polymeric polycarboxylic acid crosslinking agent. As used herein, the term “polymeric polycarboxylic acid” refers to a polymer having multiple carboxylic acid groups available for forming ester bonds with cellulose (i.e., crosslinks). Suitable crosslinking agents useful in forming the crosslinked fibers of the present disclosure include polyacrylic acid polymers, polymaleic acid polymers, copolymers of acrylic acid, copolymers of maleic acid, and mixtures thereof. Other suitable polymeric polycarboxylic acids include commercially available polycarboxylic acids such as polyaspartic, polyglutamic, poly(3-hydroxy)butyric acids, and polyitaconic acids. Polyacrylic acid polymers include polymers formed by polymerizing acrylic acid, acrylic acid esters, and mixtures thereof. Polymaleic acid polymers include polymers formed by polymerizing maleic acid, maleic acid esters, maleic anhydride, and mixtures thereof. Examples of suitable polyacrylic acid copolymers include poly(acrylamide-co-acrylic acid), poly(acrylic acid-co-maleic acid), poly(ethylene-co-acrylic acid), and poly(1-vinylpyrolidone-co-acrylic acid), as well as other polyacrylic acid derivatives such as poly(ethylene-co-methacrylic acid) and poly(methyl methacrylate-co-methacrylic acid). Suitable polymaleic acid copolymers include poly(methyl vinyl ether-co-maleic acid), poly(styrene-co-maleic acid), and poly(vinyl chloride-co-vinyl acetate-co-maleic acid). Suitable comonomers for forming polyacrylic and polymaleic acid copolymers include any comonomer that, when copolymerized with acrylic acid or maleic acid (or their esters), provides a polycarboxylic acid copolymer crosslinking agent that produces crosslinked cellulose fibers having the advantageous properties of bulk, absorbent capacity, liquid acquisition rate, and stable intrafiber crosslinks. Representative comonomers include, for example, ethyl acrylate, vinyl acetate, acrylamide, ethylene, vinyl pyrrolidone, methacrylic acid, methylvinyl ether, styrene, vinyl chloride, itaconic acid, and tartrate monosuccinic acid. Preferred comonomers include vinyl acetate, methacrylic acid, methylvinyl ether, and itaconic acid. Polyacrylic and polymaleic acid copolymers prepared from representative comonomers noted above are available in various molecular weights and ranges of molecular weights from commercial sources. In a preferred embodiment, the polycarboxylic acid copolymer is a copolymer of acrylic and maleic acids.

The polycarboxylic acid polymers useful in forming the crosslinked cellulose fibers include self-catalyzing polycarboxylic acid polymers. For example, self-catalyzing polycarboxylic acid crosslinking agent can include copolymers of acrylic acid or maleic acid and low molecular weight monoalkyl substituted phosphinates and phosphonates. These copolymers can be prepared with hypophosphorous acid and its salts, for example, sodium hypophosphite, and/or phosphorus acids as chain transfer agents. The polycarboxylic acid polymers and copolymers can be used alone, in combination, or in combination with other crosslinking agents known in the art.

In some embodiments, the polymeric polycarboxylic acid crosslinking agents can be used with a crosslinking catalyst to accelerate the bonding reaction between the crosslinking agent and the cellulose fiber to provide the crosslinked cellulose fibers. Suitable crosslinking catalysts include any catalyst that increases the rate of ester bond formation between the polycarboxylic acid crosslinking agent and cellulose fibers. For example, crosslinking catalysts include alkali metal salts of phosphorous containing acids such as alkali metal hypophosphites, alkali metal phosphites, alkali metal polyphosphonates, alkali metal phosphates, and alkali metal sulfonates.

In some embodiments, suitable crosslinking agents for making crosslinked cellulose fibers are bifunctional which are capable of bonding with the hydroxyl groups, and create covalently bonded bridges between hydroxyl groups on the cellulose molecules within the fiber. The crosslinking agents include polycarboxylic acids or selected from urea derivatives such as methylolated urea, methylolated cyclic ureas, methylolated lower alkyl substituted cyclic ureas, methylolated dihydroxy cyclic ureas. Preferred urea derivative crosslinking agents would be dimethyloldihydroxyethylene urea (DMDHEU), dimethyldihydroxyethylene urea. Mixtures of the urea derivatives may also be used. Preferred polycarboxylic acid crosslinking agents are citric acid, tartaric acid, malic acid, succinic acid, glutaric acid, or citraconic acid. These polycarboxylic crosslinking agents are particularly useful when the proposed use of the paperboard is food packaging. Other polycarboxylic crosslinking agents that may be used are poly(acrylic acid), poly(methacrylic acid), poly(maleic acid), poly(methylvinylether-co-maleate) copolymer, poly(methylvinylether-co-itaconate) copolymer, maleic acid, itaconic acid, and tartrate monosuccinic acid. Mixtures of the polycarboxylic acids may also be used. The crosslinking agent can include a catalyst to accelerate the bonding reaction between the crosslinking agent and the cellulose molecule, but most crosslinking agents do not require a catalyst. Suitable catalysts include acidic salts that can be useful when urea-based crosslinking substances are used. Such salts include ammonium chloride, ammonium sulfate, aluminum chloride, magnesium chloride, or mixtures of these or other similar compounds. Alkali metal salts of phosphorus containing acids may also be used.

Other crosslinking agents are described in Chung U.S. Pat. No. 3,440,135; Lash et al. U.S. Pat. No. 4,935,022; Herron et al. U.S. Pat. No. 4,889,595; Shaw et al. U.S. Pat. No. 3,819,470; Steijer et al. U.S. Pat. No. 3,658,613; Dean et al. U.S. Pat. No. 4,822,453; and Graef et al. U.S. Pat. No. 4,853,086, all of which are in their entirety incorporated herein by reference.

In some embodiments, polyacrylic acid crosslinked cellulosic fibers can be prepared by applying polyacrylic acid to the cellulosic fibers in an amount sufficient to effect intrafiber crosslinking. The amount applied to the cellulosic fibers can be from about 1 to about 10 percent by weight based on the total weight of fibers. In one embodiment, crosslinking agent in an amount from about 4 to about 6 percent by weight based on the total weight of dry fibers. In some embodiments, polyacrylic acid crosslinked cellulosic fibers can be prepared using a crosslinking catalyst. Suitable catalysts can include acidic salts, such as ammonium chloride, ammonium sulfate, aluminum chloride, magnesium chloride, magnesium nitrate, and more preferably alkali metal salts of phosphorous-containing acids, like phosphoric, polyphosphoric, phosphorous and hypophosphorous acids. In one embodiment, the crosslinking catalyst is sodium hypophosphite. The amount of catalyst used can vary from about 0.1 to about 5 percent by weight based on the total weight of dry fibers.

In certain embodiments, the crosslinked cellulosic fibers can include crosslinked rayon or lyocell derivatives.

The cellulosic fibers useful for crosslinked cellulosic fibers can be derived primarily from wood pulp. Suitable wood pulp fibers can be obtained from well-known chemical processes such as the kraft and sulfite processes, with or without subsequent bleaching. The pulp fibers may also be processed by thermomechanical, chemithermomechanical methods, or combinations thereof. The preferred pulp fiber is produced by chemical methods. Ground wood fibers, recycled or secondary wood pulp fibers, and bleached and unbleached wood pulp fibers can be used. A preferred starting material is prepared from long-fiber coniferous wood species, such as southern pine, Douglas fir, spruce, and hemlock. Details of the production of wood pulp fibers are well-known to those skilled in the art. Suitable fibers are commercially available from a number of companies, including International Paper Company. For example, suitable cellulose fibers produced from southern pine that are usable in making the present disclosure are available from International Paper Company under the designations SuperSoft®, SuperSoft® Air®, and SuperSoft® Air®+.

In some embodiments, the nonwoven layer has a dry basis weight of from 15 g/m² (e.g., from 20 g/m², from 25 g/m², from 30 g/m², from 35 g/m², from 40 g/m², or from 45 g/m²) to 50 g/m² (e.g., to 45 g/m², to 40 g/m², to 35 g/m², to 30 g/m², to 25 g/m², or 20 g/m²) in the composite fabric. The composite fabric can be used, for example, as an acquisition distribution layer in an absorbent article.

In some embodiments, the crosslinked cellulose layer includes a dry basis weight of from 20 g/m² (e.g., from 40 g/m², from 60 g/m², from 80 g/m², from 100 g/m², from 120 g/m², from 140 g/m², or from 160 g/m²) to 185 g/m² (e.g., to 160 g/m², 140 g/m², 120 g/m², 100 g/m², 80 g/m², 60 g/m², or 40 g/m²) in the composite fabric. The composite fabric can be used, for example, to envelop an absorbent material in an absorbent core of an absorbent article (e.g., as a core-wrap). In some embodiments, the composite fabric can be used to sandwich an absorbent material, such that a first layer of composite fabric overlies an absorbent material, and a second layer of composite fabric underlies the absorbent material.

In some embodiments, the composite fabric in the absorbent article has a nonwoven layer at a dry basis weight of 20 g/m² or more (e.g., 30 g/m² or more, 40 g/m² or more) and/or 50 g/m² or less (e.g., 40 g/m² or less, or 30 g/m² or less), such as a dry basis weight of from 20 g/m² to 50 g/m² (e.g., from 30 g/m² to 40 g/m²) and a crosslinked cellulose layer at a dry basis weight of 70 g/m² or more (e.g., 80 g/m² or more, 90 g/m² or more, 100 g/m² or more, 110 g/m² or more) and/or 120 g/m² or less (e.g., 110 g/m² or less, 100 g/m² or less, 90 g/m² or less, or 80 g/m² or less), such as a dry basis of from 70 g/m² to 120 g/m² (e.g., from 80 g/m² to 110 g/m²). The absorbent article can include a fluid acquisition distribution layer that includes the composite fabric. For example, the composite fabric can be disposed over an absorbent core or a superabsorbent polymer. The crosslinked cellulose layer of the composite fabric can face the surface of the absorbent core. The absorbent article can have a wicking distance percentage of at least 60% after a third fluid exposure in a no load saddle wicking test when the absorbent article includes a fluid acquisition distribution layer including the composite fabric. In some embodiments, the absorbent article is a diaper or an incontinence product.

In certain embodiments, the composite fabric includes the nonwoven layer at a dry basis weight of 20 g/m² or more (e.g., 30 g/m² or more, or 40 g/m² or more) to 50 g/m² or less (e.g., 40 g/m² or less, or 30 g/m² or less) and the crosslinked cellulose layer at a dry basis weight of 40 g/m² or more (e.g., 50 g/m² or more, 60 g/m² or more) and/or 70 g/m² or less (e.g., 60 g/m² or less, or 50 g/m² or less). In some embodiments, the composite fabric includes the nonwoven layer at a dry basis weight of 20 g/m² to 50 g/m² (e.g., 30 g/m² to 40 g/m²) and the crosslinked cellulose layer at a dry basis weight of 40 g/m² to less than 70 g/m² (e.g., 40 g/m² to 60 g/m², or 50 g/m²). The absorbent article can include the composite fabric, which can envelop an absorbent material in an absorbent core (e.g., as a core-wrap). For example, the composite fabric can envelop an absorbent material, such as a superabsorbent polymer, in an absorbent core. In some embodiments, the composite fabric fully envelops the absorbent material (e.g., the bulk absorbent material, such as a bulk superabsorbent polymer) in the absorbent core. In some embodiments, the composite fabric can be used to sandwich an absorbent material, such that a first layer of composite fabric overlies an absorbent material, and a second layer of composite fabric underlies the absorbent material. The crosslinked cellulose layer of the composite fabric can contact the surface of the absorbent material. The absorbent article can have a wicking distance percentage of at least 60% after a third fluid exposure in a no load saddle wicking test when the absorbent article includes the composite fabric enveloping the absorbent material. In some embodiments, the absorbent article is a diaper or an incontinence product.

Absorbent cores are described, for example, in U.S. Pat. No. 8,674,169 and PCT publication no. WO2020/046627, each of which is incorporated herein in its entirety. In some embodiments, the absorbent core can include a traditional fluff core, channeled fluff core, a complex core (e.g., a multilayered core), and/or an SAP. In some embodiments, the SAP is in the form of particles, which can be contained inside the absorbent article with the aid of an adhesive.

The composite fabric of the present disclosure can be embossed, folded, and/or perforated with one or more patterns. When used in an absorbent article, the embossing, folds, and/or perforation can physically distribute, channel, or otherwise influence the flow of a liquid insult. For example, the composite fabric can be embossed with a pattern, such as a repeated pattern. For example, the composite fabric can be pleated, folded, or otherwise have a textured surface, such that a cross section of the composite fabric has hills and valleys formed by the pleats or folds. An absorbent material, such as SAP, can be present in the valleys of the composite fabric. When the composite fabric is pleated, folded, or otherwise has a textured surface, either the nonwoven layer or the crosslinked cellulose layer can face an absorbent material of an absorbent core of the absorbent article. In some embodiments, the composite fabric can be perforated with through-openings, such as slits, channels, and/or holes.

In some embodiments, the composite fabric neutralizes odor when subjected to (e.g., wetted with) biological fluids.

In any one of the above-described embodiments, the composite fabric can be devoid of latex, latex-bonded fibers, a hydroengorged layer, a pretreated nonwoven layer, lyocell, and/or rayon.

The composite fabric of the present disclosure can be incorporated into an absorbent article, such as a personal care absorbent product, as will be described below. The personal care absorbent product can include, a diaper, an incontinence product, a feminine hygiene product, a wipe, a towel, and a tissue.

### Methods of Making the Composite Fabric

In some embodiments, the crosslinked cellulose layer is air-laid or dry-laid onto the nonwoven layer to provide the composite fabric of the present disclosure. In some embodiments, the crosslinked cellulose layer is wet-laid onto the nonwoven layer. The crosslinked cellulose fibers from the crosslinked cellulose layer can be hydro-entangled into polymeric fibers and/or filaments from the nonwoven layer in the interfacial region. For example, in a hydro-entangling process, the hydro-entanglement water jets first contact the cellulosic fibers and drive the cellulosic fibers into the nonwoven polymeric fibers. Hydro-entangling processes are described, for example, in U.S. Publication No. 2018/0326699 and CA patent no. 841,938, each of which is incorporated herein by reference in its entirety.

The hydroentangling step causes the different fiber types to be entangled by the action of a plurality of thin jets of high-pressure water impinging on the fibers. The fine mobile spun-laid filaments are twisted around and entangled with themselves and with the other fibers, which gives a material with a very high strength in which all fiber types are intimately mixed and integrated. Entangling water is drained off through the forming fabric, and can be recycled, if desired after purification. The energy supply needed for the hydroentangling is relatively low, i.e., the material is easy to entangle.

A hydroentangling process for forming a fabric occurs by mechanically wrapping and knotting fibers in a web about each other through the use of high velocity jets of water. The process uses fine, high velocity jets of water to impact a fibrous web and cause the fibers to curl and entangle about each other. The water jets perforate the web and entangle the fibers, producing fabrics that reflect the pattern of a forming belt which carries the web under the water jets. This produces a fabric with a textile fabric appearance and good drapability. A binder can be added to some hydroentangled fabrics to increase their strength and dimensional stability to make them liquid repellant. The process can be used on dry-laid webs and on wet laid webs. A lower energy hydroentangling process, using lower velocity water jets, can provide a product that has less entanglement, which can optionally include a binder. The hydroentangling process is described, for example, in The Nonwovens Fabric Handbook published by Association of the Nonwoven Fabrics Industry (INDA), Cary N.C. 1999, herein incorporated by reference in its entirety.

Examples of “laying” processes include wet-laying and air-laying (the latter occasionally also referred to as dry-laying). Example dry-laying processes include but are not limited to air-laying, carding, and combinations thereof typically forming layers. Examples of combinations include but are not limited to spunbond-meltblown-spunbond (SMS), spunbond-carded (SC), spunbond-airlaid (SA), meltblown-airlaid (MA), and combinations thereof, typically in layers. Combinations which include direct extrusion can be combined at about the same point in time as the direct extrusion process (e.g., spinform and coform for SA and MA), or at a subsequent point in time. In the above examples, one or more individual layers can be created by each process. For instance, SMS can mean a three layer, ‘sms’ web, a five layer ‘ssmms’ web, or any reasonable variation thereof wherein the lower case letters designate individual layers and the upper case letters designate the compilation of similar, adjacent layers.

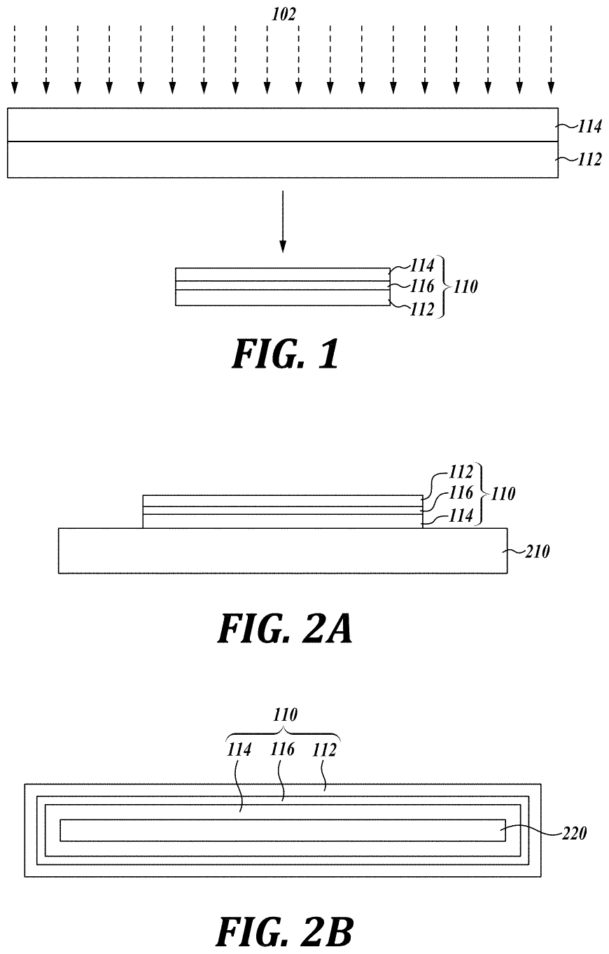

FIG. 1 shows a hydro-entangling process for entangling crosslinked cellulose fibers into a nonwoven material, which can be in the form of a fabric or fibers. Referring to FIG. 1, crosslinked cellulose fibers 114 is provided onto a nonwoven material 112, and water jets 102 are directed toward the crosslinked cellulose fibers to push the cellulose fibers into the nonwoven material, thereby providing composite fabric 110. The water jet pressure can be varied, such that at higher water pressures, the degree of crosslinked cellulose fiber penetration into the nonwoven material increases, and interfacial region 116 can increase in thickness.

In some embodiments, the present disclosure features a method of making a composite fabric, including supplying polymeric fibers and/or filaments; supplying crosslinked cellulose fibers; air-laying or wet-laying the crosslinked cellulose fibers to provide a crosslinked cellulose layer on a nonwoven layer of polymeric fibers and/or filaments, wherein the crosslinked cellulose layer is positioned opposed to the nonwoven layer (e.g., without an intervening layer different from the crosslinked cellulose layer and the nonwoven layer; in some embodiments, the crosslinked cellulose layer is immediately adjacent to the nonwoven layer); and physically entangling the polymeric fibers and/or filaments from the nonwoven layer and the crosslinked cellulose fibers from the crosslinked cellulose layer to provide an interfacial region between the nonwoven layer and the crosslinked cellulose layer, wherein the nonwoven layer and the crosslinked cellulose layer are mechanically inseparable in a dry state.

In some embodiments, physically entangling the polymeric fibers and/or filaments from the nonwoven layer and the crosslinked cellulose fibers from the crosslinked cellulose layer includes hydro-entangling the crosslinked cellulose fibers into the polymeric fibers and/or filaments. The polymeric fibers and/or filaments can be in the form of a bonded carded web fabric, a carded web, a spunbond fabric, a melt blown fabric, or any combination thereof. In some embodiments, the polymeric fibers are synthetic.

In some embodiments, the nonwoven layer is a top layer, and the crosslinked cellulose layer is a bottom layer. In certain embodiments, the nonwoven layer is a bottom layer, and the crosslinked cellulose layer is a top layer. The crosslinked cellulose layer can pre-formed prior to entangling with the nonwoven layer. In some embodiments, the crosslinked cellulose layer is not pre-formed prior to entangling with the nonwoven layer, and/or the nonwoven layer is not pre-formed prior to entangling with the crosslinked cellulose layer. In certain embodiments, the nonwoven layer can be pre-formed, or formed in situ, during the entangling process.

This present disclosure combines the integrity of nonwovens and absorbency of crosslinked cellulose fiber together to offer both excellent fluid management capability and physical characteristics such as resiliency/bunching free.

### Absorbent Articles

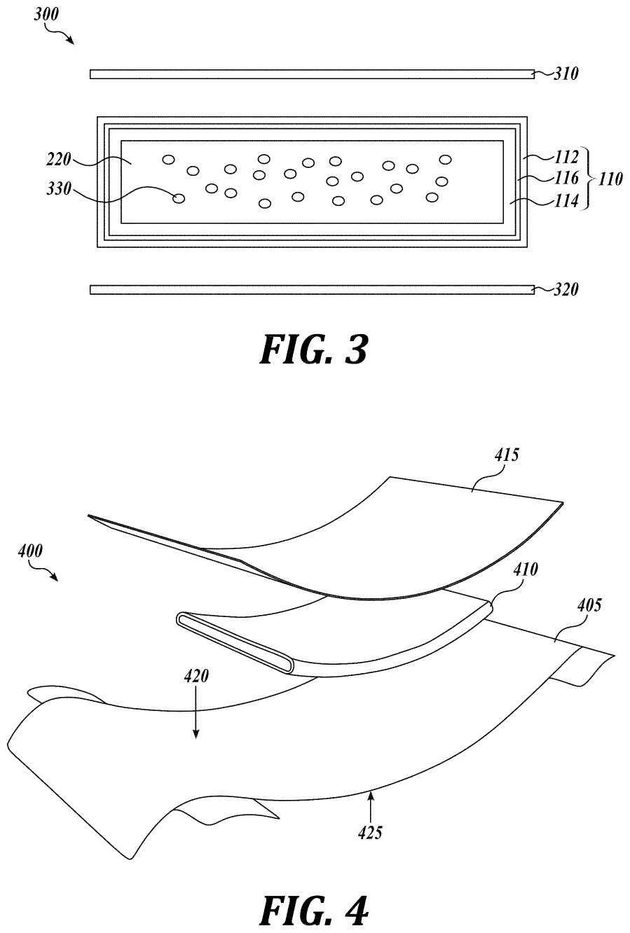

The composite fabric of the present disclosure can be used in an absorbent article. Referring to FIG. 2A, composite fabric 110 can be used in an absorbent article as a fluid acquisition and distribution layer (ADL) over absorbent material 210 that can include, for example, fluff or an SAP. The composite fabric 110 can be disposed over an absorbent core that includes fluff or a superabsorbent polymer, and the crosslinked cellulose layer 114 faces and/or contacts the surface of the absorbent material. In some embodiments, referring to FIG. 2B and FIG. 3, the composite fabric 110 of the present disclosure can be used to envelop absorbent material 220 (e.g., as a core-wrap around absorbent material 220), where the crosslinked cellulose layer 114 faces and/or contacts the surface of absorbent material 220. In some embodiments, the composite fabric can be used to sandwich an absorbent material, such that a first layer of composite fabric overlies an absorbent material, and a second layer of composite fabric underlies the absorbent material. Absorbent material 220 can include a fluff pulp (i.e., fluff), high-loft through air bonded carded web (TABCW), and/or an SAP 330. In some embodiments, absorbent material 220 can include a highly densified fluff pulp and SAP. As shown in FIG. 3, the enveloped absorbent material can be sandwiched between a liquid permeable topsheet 310 and a backsheet 320 to provide absorbent article 300. Backsheet 320 can be liquid-impermeable.

In some embodiments, the absorbent material includes 30% or more (e.g., 40% or more, 50% or more, 60% or more, 70% or more, 80% or more) and/or 90% or less (e.g., 80% or less, 70% or less, 60% or less, 50% or less, or 40% or less) by weight of the absorbent synthetic polymer and 10% or more (e.g., 20% or more, 30% or more, 40% or more, 50% or more, 60% or more) and/or 70% or less (e.g., 60% or less, 50% or less, 40% or less, 30% or less, or 20% or less) by weight of the fluff pulp. In some embodiments, the absorbent material can include a highly densified mixture of fluff pulp and SAP.

When the composite fabric is used as the ADL, as an envelope around, or otherwise sandwiches an absorbent material, improved fluid management can be observed in the absorbent articles, compared to an absorbent article that includes conventional ADL or core-wrap materials, or compared to an absorbent article having one of the nonwoven layer or the crosslinked cellulose layer, or a combination of a non-entangled nonwoven layer and crosslinked cellulose layer.

In some embodiments, when the absorbent material includes an SAP, the SAP can be in the form of particles held inside the absorbent article by the fabric with the aid, for example, of an adhesive.

When the composite fabric wraps around an absorbent material (e.g., fluff and/or the SAP) to provide an absorbent core (e.g., FIGS. 2B and 3), the absorbent material can be fully wrapped or partially wrapped by composite wrap. In function, the composite fabric can also serve as the fluid acquisition distribution layer in this simplified design.

The absorbent article can include a personal care absorbent product. For example, the personal care absorbent product can include a diaper, an incontinence product, a feminine hygiene product (e.g., a sanitary napkin, a panty liner), a wipe, a towel, and/or a tissue. In certain embodiments, the absorbent article is a diaper, an incontinence product, or a feminine hygiene product.

In some embodiments, the absorbent article of the present disclosure has an intake time decrease of at least 23% from a first fluid exposure to a second subsequent fluid exposure in a flat acquisition under load test, when the absorbent article includes a fluid acquisition distribution layer including the composite fabric.

In some embodiments, the absorbent article has an intake time decrease of at least 25% from a first fluid exposure to a second subsequent fluid exposure in a flat acquisition under load test, when the absorbent article includes the composite fabric enveloping the absorbent material.

In some embodiments, the absorbent article has an intake time decrease of at least 8% from a second fluid exposure to a third subsequent fluid exposure in a flat acquisition under load test, when the absorbent article includes a fluid acquisition distribution layer including the composite fabric.

In some embodiments, the absorbent article has an intake time decrease of at least 12% from a second fluid exposure to a third subsequent fluid exposure in a flat acquisition under load test, when the absorbent article includes the composite fabric enveloping the absorbent material.

In some embodiments, the absorbent article has a wicking distance percentage of at least 60% after a third fluid exposure in a no load saddle wicking test when the absorbent article includes a fluid acquisition distribution layer including the composite fabric.

In some embodiments, the absorbent article has a wicking distance percentage of at least 60% after a third fluid exposure in a no load saddle wicking test when the absorbent article includes the composite fabric enveloping the absorbent material.

In some embodiments, the absorbent article has a rewet amount less than 0.5 g from a first fluid exposure in a flat acquisition under load test when the absorbent article includes a fluid acquisition distribution layer including the composite fabric, or the composite fabric enveloping the absorbent material.

In some embodiments, the absorbent article has a rewet amount less than 0.5 g from a second fluid exposure in a flat acquisition under load test when the absorbent article includes a fluid acquisition distribution layer including the composite fabric.

In some embodiments, the absorbent article has a rewet amount less than or equal to 0.8 g from a second fluid exposure in a flat acquisition under load test when the absorbent article includes the composite fabric enveloping the absorbent material.

In some embodiments, the absorbent article has a rewet amount increase of less than 11.9 g from a second fluid exposure to a third subsequent fluid exposure in a flat acquisition under load test when the absorbent article includes a fluid acquisition distribution layer including the composite fabric.

In some embodiments, the absorbent article has a rewet amount increase of less than 0.35 g from a first fluid exposure to a second subsequent fluid exposure in a flat acquisition under load test when the absorbent article includes a fluid acquisition distribution layer including the composite fabric.

In some embodiments, the absorbent article has a rewet amount increase of less than 4.42 g from a second fluid exposure to a third subsequent fluid exposure in a flat acquisition under load test when the absorbent article includes the composite fabric enveloping the absorbent material.

In some embodiments, the absorbent article has a rewet amount increase of less than 0.73 g from a first fluid exposure to a second subsequent fluid exposure in a flat acquisition under load test when the absorbent article includes the composite fabric enveloping the absorbent material.

An exemplary rewet amount range per fluid exposure for some embodiments of diapers including an ADL or the composite fabric enveloping the absorbent material is shown in Table 1.

TABLE 1
Rewet amount per fluid exposure for diapers including an ADL
or core-wrap composite fabric.

| | | Composite as |
|---|---|---|
| | Composite as ADL | Core-Wrap |
| Fluid Exposure (#) | Rewet (grams) | Rewet (grams) |
| 1 | 0.09-0.28 | 0.07-0.4 |
| 2 | 0.1-0.41 | 0.08-0.8 |
| 3 | 0.1-12 | 0.09-4.5 |

Feminine Hygiene Product

The composite fabric of the present disclosure can be used in an absorbent article, such as a feminine hygiene product (e.g., a sanitary napkin, a panty liner). The feminine hygiene product can include a composite fabric including a nonwoven layer including polymeric fibers and/or filaments; a crosslinked cellulose layer including crosslinked cellulose fibers, wherein the crosslinked cellulose layer is positioned opposed to the nonwoven layer; and an interfacial region between the nonwoven layer and the crosslinked cellulose layer, including physically entangled polymeric fibers and/or filaments from the nonwoven layer and crosslinked cellulose fibers from the crosslinked cellulose layer, wherein the nonwoven layer and the crosslinked cellulose layer are mechanically inseparable in a dry state.

The feminine hygiene product can include an absorbent core including an absorbent material. In some embodiments, the composite fabric is disposed over the absorbent core. In some embodiments, the composite fabric envelops at least a portion of the absorbent material. In some embodiments, the composite fabric can be used to sandwich the absorbent material, such that a first layer of composite fabric overlies an absorbent material, and a second layer of composite fabric underlies the absorbent material.

When subjected to a fluid insult, the composite fabric distributes the fluid to a front portion, a middle portion, and a back portion of the feminine hygiene product. In some embodiments, when subjected to fluid insult, the front portion, middle portion, and back portion of the composite fabric of the feminine hygiene product each includes an amount of fluid within 20 wt % to 45 wt % of each portion. As used herein, the middle portion is 7.5 cm in length and is situated between the front and back portions, with the remaining length equally divided between the front and back portions.

Absorbent Article Configurations—Absorbent Core

The composite fabric of the present disclosure can be included in absorbent articles and can serve, among other purposes, as acquisition-distribution layers (ADL), or used to wrap at least partially around an absorbent material, which can be or include one or more of a number of absorbent materials. Various exemplary configurations of the “core-wrap” absorbent articles are described in reference to FIGS. 4-8C in the forthcoming paragraphs.

FIG. 4 is a schematic diagram illustrating an example absorbent article 400, in accordance with embodiments of the present disclosure. In some embodiments, the example absorbent article 400 includes: a backsheet 405, an absorbent core 410, and a topsheet 415. The example absorbent article 400 is structured to receive a liquid insult via the topsheet 415, to distribute the liquid through the absorbent core 410, and to absorb the liquid, while inhibiting the liquid from circumventing the backsheet 405, thereby reducing or eliminating wetness, discomfort, and/or irritation from being experienced by a wearer of the absorbent article 400. Example absorbent article 400 is an example of absorbent article 300 described in reference to FIG. 3.

In some embodiments, the backsheet 405 includes constituent materials that are impermeable to liquids, such as one or more layers of polymeric, elastomeric, and/or metallic material creating a liquid-impermeable barrier. Conversely, the topsheet 415 can include materials that are permeable to liquids, such that a liquid insult incident on the topsheet 415 can be wicked, channeled, or otherwise pass through the topsheet 415 to the absorbent core 410 with negligible physical resistance. When assembled, the topsheet 415 can overlie the absorbent core 410 and can contact the inner surface of the backsheet 405. In this way, contacting the inner surface of the backsheet 405 can include contacting the backsheet 405 at one or more points, around the periphery of the absorbent core 410 and/or coextensive with the backsheet 405. The various configurations can permit the absorbent article to bend or twist without significant bunching or squeezing of the absorbent core 410.

The backsheet 405 can define an inner surface 420 and an outer surface 425. The inner surface 420 can be or include physical clasps, latches, tabs, adhesives or another configuration whereby the backsheet 405 can mechanically couple with the absorbent core 410 and/or the topsheet 415, and whereby the backsheet 405 can removably couple with a garment of the wearer. In some embodiments, the absorbent article can be in a pant form, without any fasteners. For example, the absorbent core 410 can be disposed on the inner surface 420 of the backsheet 405, and can be retained, held, fixed, or otherwise mechanically coupled with the backsheet 405. In some embodiments, the backsheet 405 and the topsheet 415 together define a pocket into which the absorbent core 410 can be removably disposed. In this way, the absorbent article 400 can be reusable or can be disassembled to facilitate disposal of compostable materials and recycling of plastic components.

In some embodiments, the backsheet 405, the topsheet 415, the absorbent core 410, and the composite fabric of the present disclosure can be embossed folded, pleated, and/or perforated to physically distribute, channel, or otherwise influence the flow of a liquid insult incident on the topsheet 415, wherein the folded or pleated composite fabric optionally includes an absorbent material within the folds or pleats. When the composite fabric is pleated, folded, or otherwise has a textured surface, either the nonwoven layer or the crosslinked cellulose layer can face an absorbent material of an absorbent core of the absorbent article.

In some embodiments, the topsheet 415 is textured to improve the sensation of the wearer while donning the absorbent article. In an illustrative example, the texture and/or pattern can include one or more pores to improve circulation of air through the absorbent article 400, thereby reducing humidity near the surface of the skin of the wearer and sequestering and/or denaturing odiferous gases. Similarly, the topsheet 415 can include a micro-textured surface to impart a soft feeling to the surface, without altering the liquid permeability or porosity of the topsheet 415.

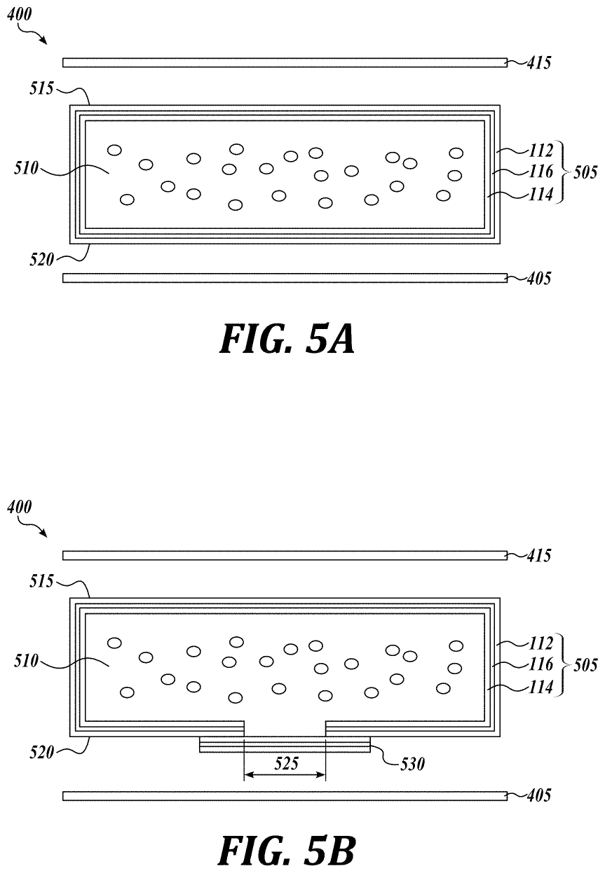

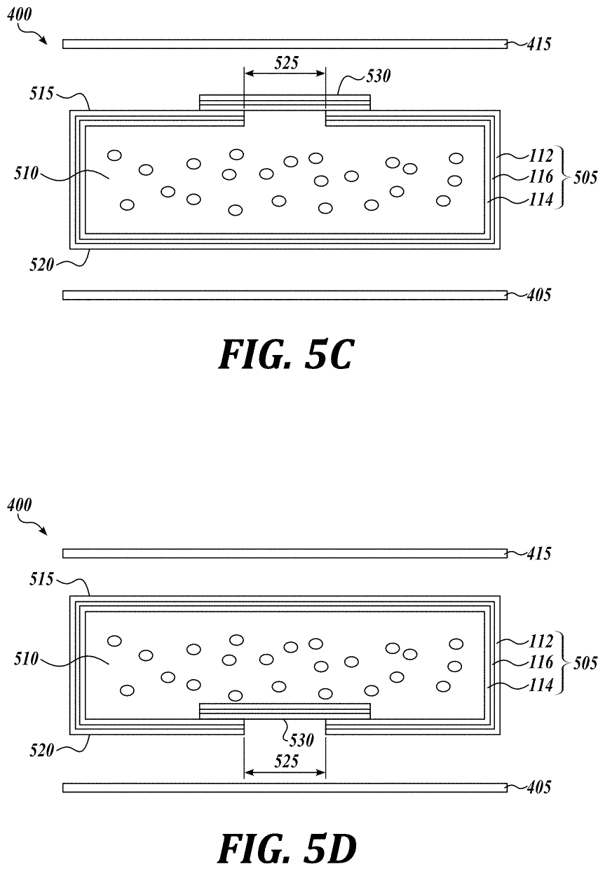

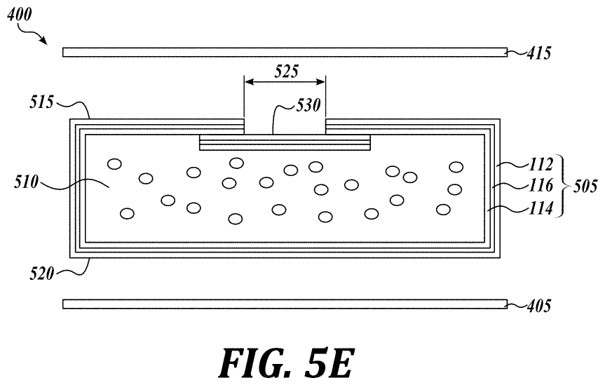

Referring to FIGS. 5A-5E, various configurations of core-wrap absorbent articles are described. FIG. 5A illustrates one example of the constituent materials and configurations contemplated. FIGS. 5B-8C illustrate additional and/or alternative configurations and/or materials that can be included in embodiments of the absorbent articles.

FIG. 5A is a schematic diagram illustrating internal structures of the example absorbent article 400 of FIG. 4, in accordance with embodiments of the present disclosure. The example absorbent article 400 includes, as constituents of the absorbent core 410, a distribution layer 505, which can include or be formed of the composite fabric of the present disclosure, disposed surrounding at least a portion of an absorbent material 510. The distribution layer 505 and the absorbent material 510 can together act to distribute and absorb a liquid insult incident on the topsheet 415 and to reduce rewetting subsequent initial absorption.

In some embodiments, the absorbent material 510 defines an upper surface 515 and a lower surface 520 of the absorbent core 410. The distribution layer 505, in turn, surrounds at least a portion of the upper surface 515 and the lower surface 520. The distribution layer 505 can fully surround the upper surface 515 and the lower surface 520 of the absorbent core 410. For example, the distribution layer 505 can be or include a rectangular-planar material having four edges that is wrapped around the absorbent material 510 such that two edges contact each other along the lower surface 520 or along the upper surface 515 of the absorbent core 410.

The distribution layer 505 can be or include composite fabric 110 including two or more constituent layers. The constituent layers can include nonwoven layer 112 and a crosslinked cellulose layer 114. The nonwoven layer 112 can be or include polymeric fibers and/or filaments, as described in more detail in reference to the preceding figures. In contrast, the crosslinked cellulose layer 114 can be or include crosslinked cellulose fibers.

The crosslinked cellulose layer 114 can be positioned opposed to the nonwoven layer 112 and can define the interfacial region 116 between the nonwoven layer 112 and the crosslinked cellulose layer 114, as described in more detail in reference to FIGS. 1-3. The interfacial region 116 can include physically entangled polymeric fibers and/or filaments from the nonwoven layer 112 and crosslinked cellulose fibers from the crosslinked cellulose layer 114. In this way, the nonwoven layer 112 and the crosslinked cellulose layer 114 can be mechanically inseparable in a dry state.

Referring to FIG. 5B and FIG. 5C, alternatively, the distribution layer 505 can define a gap 525 on the upper surface 515 or the lower surface 520 of the absorbent core 410. Where the gap 525 can retain liquid or can otherwise impair the distribution of liquid through the distribution layer 505, the absorbent core 410 can further include a cover distribution layer 530 disposed over the gap 525. The cover distribution layer 530 can overlie at least a portion of the distribution layer 505, such that the distribution layer 505 is disposed between at least a portion of the cover distribution layer 530 and the absorbent material 510. In terms of assembly, the distribution layer 505 can be wrapped around the portion of the absorbent material 510, defining the gap 525 on the upper surface 515 or the lower surface 520, and can be coupled by pressure, adhesive, physical closures, or other approaches, over which the cover distribution layer 530 can be physically coupled with the distribution layer 505 by similar techniques. In some embodiments, the cover distribution layer 530 is or includes the composite fabric 110, such that where the cover distribution layer 530 contacts the absorbent material 510, it serves to distribute liquid in a manner similar to the distribution layer 505.

Referring to FIG. 5D and FIG. 5E, the cover distribution layer 530 can underlie at least a portion of the distribution layer 505, such that the cover distribution layer 530 is disposed between at least a portion of the distribution layer 505 and the absorbent material 510. In terms of assembly, the cover distribution layer 530 can be physically coupled with the absorbent material 510 by pressure, adhesive, physical closures, or other approaches, over which the distribution layer 505 can be wrapped around the portion of the absorbent material 510, defining the gap 525 on the upper surface 515 or the lower surface 520, and thereby can be coupled with the cover distribution layer 530 and the absorbent material 510.

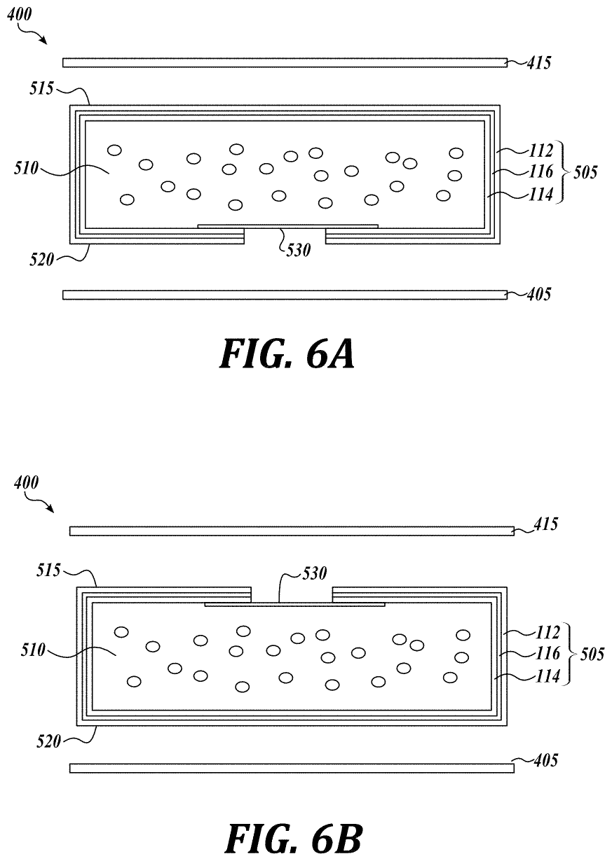

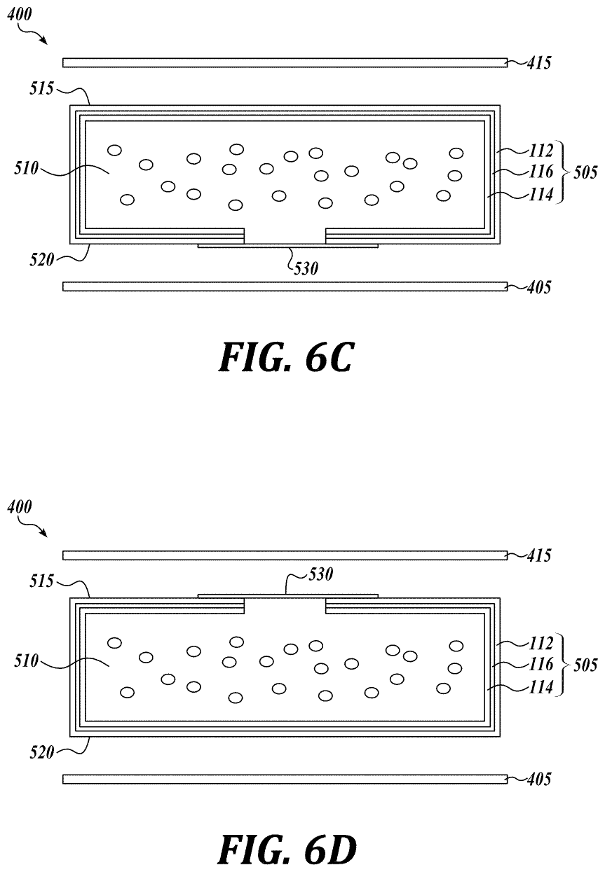

Referring to FIGS. 6A-6D, the cover distribution layer 530 can be or include a spunbond meltblown spunbond (SMS) material, a spunbound (SB) material, spunbond-carded (SC), spunbond-airlaid (SA), meltblown-airlaid (MA), or combinations thereof, as described previously. As described in reference to FIGS. 5B-5E, the SMS and SB materials can be disposed overlying at least a portion of the distribution layer 505 or underlying the distribution layer 505, and can be disposed on the upper surface 515 or the lower surface 520, corresponding to the position of the gap 525 on the absorbent core 410.

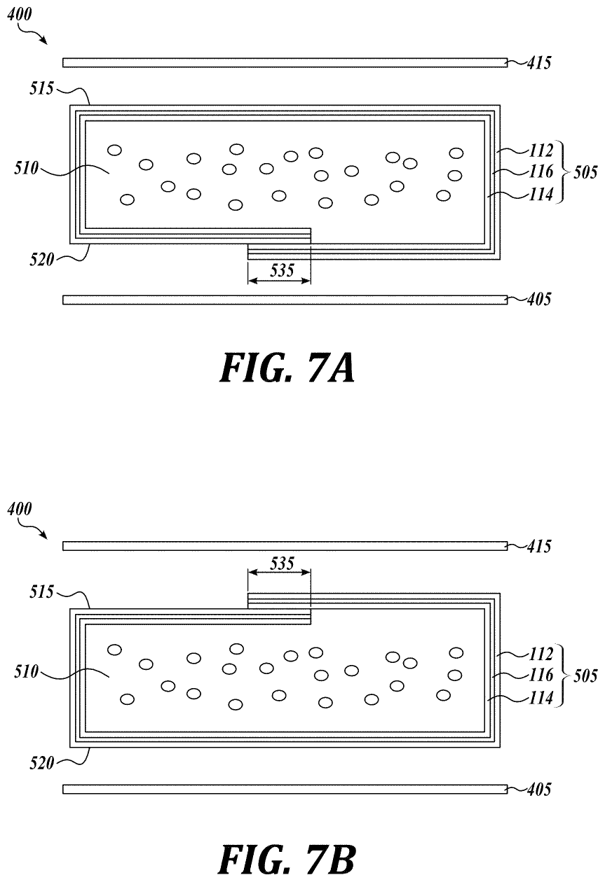

Referring to FIG. 7A and FIG. 7B, in some embodiments, the distribution layer 505 overlaps on the upper surface 515 or the lower surface 520 of the absorbent core 410 by at least a portion 535 of a width of the distribution layer 505. In the example of the rectangular-planar material, the two edges can overlap on the upper surface 515 or on the lower surface 520. Advantageously, the configurations including the overlapping portion can be manufactured with fewer processes, rather than including the steps involved in preparing and disposing the cover distribution layer 530.

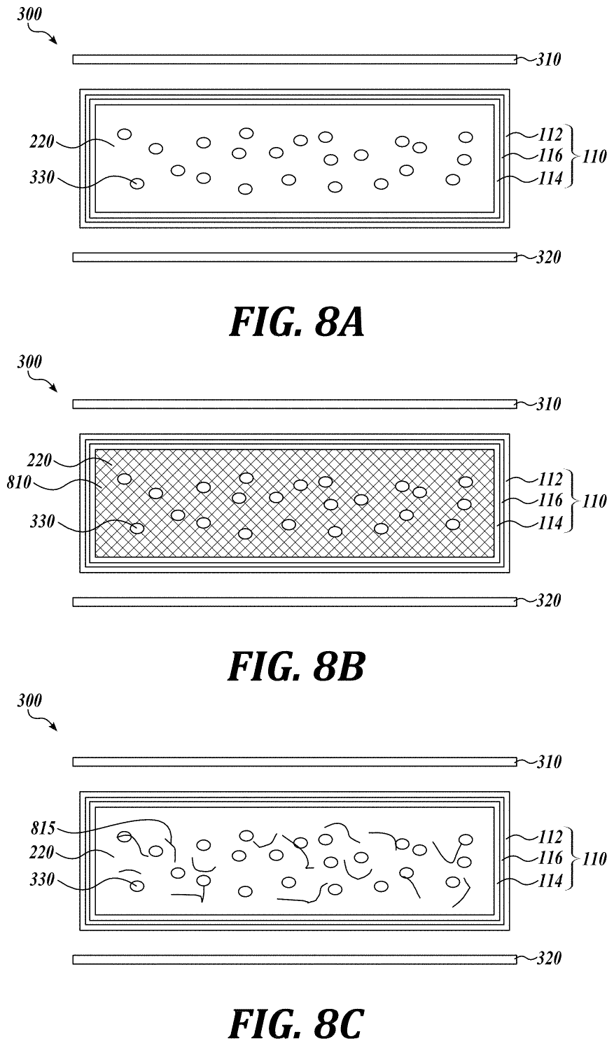

Referring to FIG. 8A, FIG. 8B, and FIG. 8C, absorbent materials 220 are described in reference to absorbent article 300, as may also be included as part of example article 400 of FIG. 4. The absorbent material 220 in the absorbent core can be or include one or more constituent materials selected to provide improved absorbance, wicking, and/or retention properties of the absorbent article 300. For example, the absorbent material 220 can be or include a synthetic absorbent polymer 330 and a high-loft through air bonded carded web (TABCW) 810. In another example, the absorbent material 220 can be or include an absorbent synthetic polymer 330 and a fluff pulp 815. The absorbent material 220 can include the aforementioned materials in combination. In some embodiments, the absorbent material 220 includes from 30% to 90% by weight of the absorbent synthetic polymer 330 and from 10% to 70% by weight of the fluff 815. The composition of the absorbent material can be determined at least in part by a balance of absorbency, weight, density, and other wetting properties, as described in reference to the absorbent article test procedures, below. For example, while absorbent synthetic polymer 330 can exhibit increased retention, fluff 815 can improve acquisition and wicking. In this way, overall performance of the absorbent article can depend on the specific application, for example when wicking can be more desirable, as when relatively high volumes of liquid are to be absorbed quickly, as opposed to applications where volumes are relatively low but are to be absorbed steadily over a period of time.

In this way, the absorbent material 220 can include from 5% to 99% by weight of the absorbent synthetic polymer 330 and from 1% to 95% by weight of the fluff 815, from 10% to 90% by weight of the absorbent synthetic polymer 330 and from 10% to 90% by weight of the fluff 815, from 15% to 90% by weight of the absorbent synthetic polymer 330 and from 10% to 85% by weight of the fluff 815, from 20% to 90% by weight of the absorbent synthetic polymer 330 and from 10% to 80% by weight of the fluff 815, from 25% to 90% by weight of the absorbent synthetic polymer 330 and from 10% to 75% by weight of the fluff 815, from 30% to 90% by weight of the absorbent synthetic polymer 330 and from 10% to 70% by weight of the fluff 815, from 35% to 90% by weight of the absorbent synthetic polymer 330 and from 10% to 65% by weight of the fluff 815, from 40% to 90% by weight of the absorbent synthetic polymer 330 and from 10% to 60% by weight of the fluff 815, from 45% to 90% by weight of the absorbent synthetic polymer 330 and from 10% to 55% by weight of the fluff 815, from 50% to 90% by weight of the absorbent synthetic polymer 330 and from 10% to 50% by weight of the fluff 815, from 55% to 90% by weight of the absorbent synthetic polymer 330 and from 10% to 45% by weight of the fluff 815, from 60% to 90% by weight of the absorbent synthetic polymer 330 and from 10% to 40% by weight of the fluff 815, from 65% to 90% by weight of the absorbent synthetic polymer 330 and from 10% to 40% by weight of the fluff 815, from 70% to 90% by weight of the absorbent synthetic polymer 330 and from 10% to 30% by weight of the fluff 815, from 75% to 90% by weight of the absorbent synthetic polymer 330 and from 10% to 25% by weight of the fluff 815, from 80% to 90% by weight of the absorbent synthetic polymer 330 and from 10% to 20% by weight of the fluff 815, from 85% to 90% by weight of the absorbent synthetic polymer 330 and from 10% to 15% by weight of the fluff 815, including fractions or interpolations thereof.

### Absorbent Article Test Procedures

No Load Saddle Wicking for Absorbent Articles

This test determines how quickly an absorbent hygiene product can absorb a certain amount of fluid while in constrained in a “U” shaped saddle simulating the position of the absorbent article when in human use. Additionally, the test determines the distance wicked by the fluid after all doses of fluid. This test assesses an absorbent article's fluid intake and fluid distribution capabilities in a configuration similar to real life usage.

Equipment and Materials Needed

Equipment and materials needed for this test are as follows: Ruler, simulated urine (0.9% saline solution), saddle device, peristaltic pump with dispensing tubing that has attachment to prevent dispensing tube from touching the diaper, timer, stopwatch, magnetic board, and 4 magnets.

Sample Preparation

1. Determine dimensions of sample.

2. If testing infant products, then measure product length and width.

3. Mark center of sample length.

4. Measure 9 cm towards the front of the sample and mark with an “X” ensuring the “X” is centered in reference to the absorbent core width. The “X” will be the insult point.

5. Optionally, the elastic leg gathers of the diaper may be cut for ease of testing as long as the cut does not interfere with the absorbent capabilities of the diaper.

Calibration

1. Prepare the appropriate amount of 0.9% saline solution for testing in a container that can fit the inlet of the testing pump.

2. Set pump to desired flow rate and dose volume.

Infant products should have a rate of (900 ml/minute) and a dose of 85 ml.

3. Dispense 1 dose into a graduated cylinder. If the dose is incorrect, then calibrate the tubing.

Testing Procedure

1. Place dispensing tube perpendicular to insult point and as close as possible to the absorbent article surface without touching the surface with the dispensing point.

2. Start peristaltic pump, stopwatch, and timer (set to 20 minutes) simultaneously.

3. Stop stopwatch when fluid is absorbed.

4. When 20 minute timer ends, repeat steps 1-3 two more times.

5. After the third round of the 20 minute timer ending, remove the absorbent article and stretch out flat on a magnetic board and secure the absorbent article in place.

6. Measure distance fluid has wicked from the insult point towards the front and back ends of the absorbent article. To determine the wicking distance, the tester shall identify the furthest wicking distance wicked by the bulk of the fluid and exclude outlying wicking distances.

Flat Acquisition Under Load for Absorbent Hygiene Products

This test determines how quickly the absorbent hygiene product can absorb a certain amount of fluid while under high pressure, as well as how well the product retains that fluid. Thus, this test assesses an absorbent article's fluid management capabilities under load.

Equipment and Materials Needed

Equipment and materials needed for this test are as follows: magnetic board and magnets, balance with a 1,000-gram capacity sensitive to 0.01 g, ruler, simulated urine (0.9% saline solution), insult plate, rewet plate, peristaltic pump with dispensing tubing, blotter paper, weights to generate 0.38 psi, 2 timers, stopwatch.

Sample Preparation

1. Use two magnets to attach sample onto a magnetic board from either the front or back two tabs.

2. Pull the diaper taut and use two more magnets to maintain tension by holding the diaper down at the two available tabs.

3. Label the insult point which is 150 mm from the front of the absorbent core and in the center width wise of the absorbent core

Calibration

1. Prepare the appropriate amount of 0.9% saline solution for testing in a container that can fit the inlet of the testing pump.

2. Set pump to desired volume and rate.

3. Infant products should have a rate of 900 mL/min and a dose of 85 mL.

4. Dispense 1 dose into a graduated cylinder. If the dose is incorrect, then calibrate the pump.

Testing Procedure

1st Intake/Rewet

a) Place the insult board onto the product and align the front edge of the insult board to the front edge of the absorbent core. Be sure the insult point is center of the cylinder.

b) Load the board to 0.38 psi.

c) Dispense 85 ml of saline solution into the cylinder.

d) Immediately after dispensing, simultaneously start the stopwatch and the timer set to 15 minutes.

e) When all the saline is absorbed into the product, stop the stopwatch and record the acquisition time.

f) Weigh 1 dry blotter and record the weight.

g) Place the pre-weighed rewet blotter paper with the short edge aligned with the front edge of absorbent core and place the rewet plate centered over the top of the blotter paper.

h) Load the rewet plate with 0.38 psi.

i) Start the timer set to 2 minutes again.

j) After waiting 2 minutes for rewet, remove the rewet plate and rewet blotter paper.

k) Weigh blotter.

l) Measure distance wicked by fluid from the insult point towards the front and back ends of the absorbent article and record each separately as “front wicking distance” and “back wicking distance”, respectively. To determine the wicking distance, the tester shall identify the furthest wicking distance wicked by the bulk of the fluid and exclude outlying fluid wicked.

2nd Intake/Rewet

a) Follow procedure for 1st intake, except use 2 dry blotter paper for rewet.

3rd Intake/Rewet

Follow procedure for 1st intake, except use 3 dry blotter paper for rewet.

Calculations

Rewet value (g)=Blotter paper weight after rewet (g)−blotter paper weight before rewet (g).

In-Plane Radial Permeability (IPRP) Test

Permeability generally refers to the quality of a porous material that causes it to allow liquids or gases to pass through it and, as such, is generally determined from the mass flow rate of a given fluid through it. The permeability of an absorbent structure is related to the material's ability to quickly acquire and transport a liquid within the structure, both of which are key features of an absorbent article. Accordingly, measuring permeability is one metric by which a material's suitability for use in absorbent articles may be assessed. The In-Plane Radial Permeability (IPRP) of a porous material is measured according to the method described in U.S. Pat. No. 10,287,383, herein incorporated by reference in its entirety. The quantity of a saline solution (0.9% NaCl) flowing radially through an annular sample of the material under constant pressure is measured as a function of time, and testing is performed at 23° C.±2 C.° and a relative humidity 50%±5%. All samples are conditioned in this environment for twenty four (24) hours before testing.

Thickness, Bulk, and Density

This method is used to determine the single sheet thickness of material by use of a motor driven micrometer using a specified load applied for a specified time. The method is based upon TAPPI T 411.

This method is suitable for using the IPC Soft Platen technique for measuring apparent thickness. This technique employs a micrometer with pressure faces covered with soft neoprene rubber. This has the effect of reducing thickness readings due to the ability of the latex to conform to surface irregularities. This is useful when measuring materials with rough or irregular surfaces, such as linerboard and corrugating medium.

Equipment needed: Motor driven micrometer, accurate to 0.001 mm.

Wire, or other suitable calibration gauges, with thickness known to within 0.0005 mm. Gauges should extend over a range of thicknesses (e.g., 0.2-1.0 mm.)

Procedure:

Step 1.1: Clean the surfaces of the platens with lint-free paper (Bausch and Lomb Sightsaver silicon wipes) and adjust the micrometer reading to zero.

Step 1.2: With the pressure faces closed, set the reading to zero. Do not reset the zero during the following steps.

Step 1.3: Open the gap between the pressure faces and allow it to close again.

Step 1.4: Insert one of the calibration gauges and read the thickness to the nearest 0.001-mm. Repeat four times. Record each thickness reading and the average.

Step 1.5: Choose another gauge thickness and repeat Step 4. Continue for the remaining thickness gauges (a total of four different thicknesses.)

Step 1.6: Calculate the average and coefficient of variance for readings taken on each gauge. Record. Readings should agree with the calibrated gauge readings to within 0.5%. The coefficient of variance should be 0.5% or less.

Step 2.1: Follow Steps 1.1 to 1.4 for the gauge nearest to the range being worked with.

Step 2.2: Follow step 1.6.

Step 2.3: Check for parallelism of the upper and lower platens by inserting a single gauge on one side of the lower face (1-2 mm from the edge of the face) and allow the faces to close.

Record to the nearest 0.001-mm. Repeat at the edge directly opposite from this edge.

Step 2.4: Repeat Step 2.3, taking readings at positions rotated 90° from the first two (i.e., front and back edges of the lower platen if first readings were taken on the left and right edges).

Calculate the error of parallelism (P):

P=0.5[(d₁)²+(d₂)²]^{1/2}
Where: d₁=difference between readings, step 8.2.3.
   d₂=difference between readings, step 8.2.4.

Record P to the nearest 0.001-mm in the logbook.

If P>0.005 mm, the instrument should be checked by instrumentation before proceeding.

Step 3: Samples should be sufficient to obtain 50 readings (as specified in 8.6).

Step 4: Clean the surfaces of the platens with lint-free paper and adjust the micrometer reading to zero.

Step 5: Insert a single specimen into the caliper opening, allowing the pressure faces to close and the reading to stabilize. Avoid imposing any manual stress on the specimen while the reading is being made. Record the reading by manual or serial port entry using Sample Manager.

Step 6: complete 10 caliper readings in a random format (e.g., 5 readings from the outer ring 15-25 mm in and 5 reading from the center ring 15-25 mm from the center).

Step 7: do 5 readings per sheet: two in the top area, one in the middle, and two in the lower area.

After each sample, check that the instrument “zero” still reads zero.

Calculations

Calculations are done by the computer.

Step 1: To calculate air-dry bulk, cubic centimeters per gram:
Bulk, cm³/g=1000 A/B
Where: A=thickness, mm
   B=air-dry basis weight, g/m²

Step 2: To calculate air-dry (“apparent”) density, in kilograms per cubic meter:
Density, kg/m³=B/A
Where: A=thickness, mm
   B=air-dry basis weight, g/m²

Odor Control Evaluation

Method of Measuring a Reduction in Free TMA

A method of measuring a reduction of free trimethylamine (TMA) sequestered by an absorbent material, such as the composite fabric of the present disclosure or an absorbent article made therefrom. In an embodiment, the absorbent material is disposed in a closed container and is contacted with an amount of TMA. After the absorbent material has had an opportunity to sequester at least a portion of the amount TMA and, for example, the amount of TMA has reached an equilibrium between a gas headspace of the closed container and the absorbent material, a portion of the gas headspace is withdrawn from the closed container. In an embodiment, the amount of TMA is allowed to contact the absorbent material for sufficient time to reach equilibrium before a portion of the gas headspace is withdrawn from the closed container, thereby also providing sufficient time for at least a portion of the initial amount of TMA to be sequestered within the absorbent material. A person of ordinary skill in the art would readily know how to generate an equilibrium curve or other appropriate tool to monitor for and identify equilibrium.

In an embodiment, the closed container is a flexible container configured to at least partially collapse in response to the portion of the gas headspace being withdrawn. In this regard, it is easier for a user to withdraw the portion of the gas headspace from the closed container.

The withdrawn portion of the gas headspace is assayed to determine a gaseous concentration of free TMA present in the headspace. In an embodiment, measuring the amount of free TMA in the withdrawn portion includes passing the withdrawn portion of the gas headspace over a stationary phase loaded with a colorimetric marker that changes color when contacted with TMA; and measuring an amount of color change in the stationary phase in response to passing the withdrawn portion of the gas headspace over the stationary phase. In an embodiment, assaying the withdrawn portion of the gas headspace to measure the gaseous concentration of free TMA includes using a colorimetric gas detector tube, such as a Sensidyne® gas detector tube system. While colorimetric detection methods are described, it will be understood that other methods of TMA detection, for example, and not limited to, gas chromatography, can be used consistent with the methods of the present disclosure.

Reduction of free TMA is measured relative to a control. In an embodiment, the control is a null control, where a null control includes a control that does not include contacting a TMA molecule with an absorbent material. In an embodiment, the control is an absorbent material control, where an absorbent material control is an absorbent material having substantially no or no added carboxylic acid coupled to a fiber matrix (in this case, “substantially no added carboxylic acid” or “substantially free of added carboxylic acid” should be understood to mean no added carboxylic acid or an amount of added carboxylic acid between 0 wt % and 1 wt % as limited by known detection methods). As used herein, “added carboxylic acid” should be understood to mean an amount of carboxylic acid added or otherwise coupled to an absorbent material during processing or manufacturing over and above any carboxylic present in an untreated absorbent material. In an embodiment, the control absorbent material includes a fluff pulp, such as a Southern bleached softwood kraft pulp, that has not been treated with or otherwise coupled to a carboxylic acid. In this regard, a user can determine an amount of TMA reduction by the carboxylic acid coupled to the fiber matrix of the absorbent materials described herein relative to the chosen control.

In an embodiment, an amount of TMA not sequestered by the absorbent material and allowed to equilibrate within the gaseous headspace (TMA_{g}) is compared to an amount of TMA not sequestered in a control experiment that is allowed to equilibrate within the control gaseous headspace (TMA_{c}). The reduction in gaseous concentration of free TMA measured in the headspace above the absorbent material relative to that of a control may be expressed as percent reduction of free TMA (% TMA_{red}). This percent reduction can be calculated with the following equation.

% TMA_{red}=(TMA_{c}−TMA_{g}/TMA_{c})×100%

It should be noted that fluid containing TMA, such as a fluid used to insult an absorbent material or absorbent article, that resides on a side or other portion of the closed container may skew TMA reduction results. Such TMA-containing fluid that does not contact an absorbent material or absorbent article may result in increased volatilization of TMA from the TMA-containing solution into the gas headspace of the closed container. Such increased TMA volatilization may result in higher relative gaseous TMA concentrations than if the TMA-containing solution were insulted directly onto the absorbent material or absorbent article incorrectly indicating a capability (or lack thereof) of the absorbent material or absorbent article to sequester TMA.

Feminine Hygiene Product Evaluation Protocol

The feminine hygiene testing protocol generates data using standardized methods that can be used to compare the performance of one product to another. Testing includes Product Weight, Rewet Performance, and Liquid Distribution.

Measuring physical attributes such as product weight, basis weight and density provides baseline information for comparing one product to another.

Basis weight and density of an absorbent product affect liquid absorption, liquid wicking throughout the pad, and pad integrity. Basis weight and density uniformity throughout the pad or intentional profiling within portions of the pad, impact product performance.

Rewet testing provides evidence of dryness against the skin after an absorbent structure has been insulted with fluid. Rewet values are influenced by the speed liquid is absorbed into the structure, how well liquid is wicked away from the point of insult, and how well liquid is retained within the product. Liquid distribution testing quantifies the amount of fluid wicking from the point of insult out to the ends of an absorbent product. These are both important properties when analyzing absorbent feminine hygiene product performance.

Intake and rewet assays for feminine care products assess the fluid management capabilities of feminine hygiene products while laid on a flat board. Fluid used in this test is a menstrual fluid simulant. Results of the standardized assays allow comparison of products in their ability to intake fluid, mitigate rewet or flow back of fluid, and/or the distance wicked by fluid.

Equipment and Materials Needed

Equipment and materials needed for Feminine Hygiene Testing are as follows: rewet & liquid distribution template for marking the pads; basis weight—density template; filter paper, cut into 7.5 cm×6.2 cm rectangles; peristaltic pump—calibrated to 0.33 mLs/min, with 3 cams for 3 tubes; weight, rectangle, 0.46 psi or equivalent; synthetic menstrual fluid, laboratory timers, balance sensitive to 0.01 g; scissors; ruler; weighing dish; 4-250 ml plastic beakers; stainless steel tube holders; Samco Series 70 press or equivalent; position template & die cutter; cutting board; standard silicone tubing.

For intake and rewet assays, materials needed are as follow: absorbent pad, insult block; 3 or 5 ml syringe; timer; stopwatch; Synthetic Menstrual Fluid (SMF)—Processed Swine Blood (PSB)—see below for detailed process for preparation of SMF; filter paper, Whatman #3 size 11 cm; weight to generate 0.55 psi—standardized pressure setup; balance sensitive to at least 0.001 g; ruler; 250 ml plastic bottle; 50-ml test tubes.

Test Procedures:

Product Weight Variability

Step 1: Weigh all feminine hygiene products using the standard test spreadsheet and a balance to determine an average weight, standard deviation and coefficient of variation.

Step 2: As you weigh the pads, write the weight of each pad somewhere on the wrap or directly on the pad.

Step 3: Stack pads in ascending/descending order by weight.

Step 4: If wraps do not come off easily and testing will be performed with wraps on, carefully remove and weigh at least five wraps.

Step 5: Enter the individual values in the standard test spreadsheet in order to calculate adjusted average product weight.

Step 6: After weighing samples to determine adjusted average product weight, select 6-12 pads (depending on number of replicates you are testing) that have a product weight closest to the adjusted average product weight and set them aside for rewet & liquid distribution testing.

Pad Preparation for Rewet & Liquid Distribution

Step 1: Take the pads set aside for rewet & liquid distribution testing and separate them into two groups:
Group One: 3-6 pads to be tested
Group Two: 3-6 pads to be use for tare weight

Step 2: Loosen and open wrap unfolding samples so they can be laid flat with wings spread.

Step 3: Allow samples to lay flat for some time (4-8 hours) to allow them to breathe and flatten out. Applying some light weight can help to hasten this process.

Step 4: Locate the center of the sample by finding the center of the wings and mark the products for dosing.

Using Rewet & Distribution Template to Prepare for Testing

Step 1: The Rewet & Distribution Template is thin Plexiglas and has two slits that are used to mark and divide samples into three sections. A small hole in the center of the template designates the center of the template and also where it should be positioned on a feminine hygiene pad. Once this is in place over the dosing point, lines can be drawn on the pad with a marker using the slits as guides.

Position the template over the length and width of the pad aligning the center hole of the template over the center mark or dosing point of the pad.

Step 2: Mark the pad (and the wrap if applicable) using a permanent marker by tracing inside the slits of the template. This will divide the pad into three sections. If necessary, use a ruler to extend lines on the pad onto the plastic wrap.

Step 3: Label each section of the sample with a replicate number and a position identification:

For TESTING replicate #1, each section is labeled as follows: #1F (front), #1M (middle) and #1B (back). If the front of the pad cannot be discerned from the back of the pad, label as follows: #1 End-A, #1 Middle and #1 End-B). For TARE replicate #1, each section should be preceded with the letter “T” (for tare)—T #1F, T #1M, T #1.

Step 4: After marking all pads, select the ones to be used for TARE WEIGHT and cut along the lines made using the template.

Step 5: Weigh and record the weights of each section.

An average TARE weight for each section is calculated and applied to the DISTRIBUTION—PAD SECTION of the work sheet in order to determine liquid distribution. Liquid Distribution is accomplished after testing is complete when wetted sections are cut, weighed and recorded in the spreadsheet. (Wet Weight, g.—Dry Weight, g=Rewet, g).

The average of each section (Front=3.04 g, Middle=3.01 g, Back=8.59 g) is added to the Distribution—Pad Sections as “start weight, g”.

Preparing Filter Papers

Before actual testing begins, condition filter papers at ambient room temperature/humidity for at least two hours.

Count and weigh three sets of ten 7.5 cm×6.2 cm filter papers per sample being tested.

As the filter papers are weighed, write the weight (g.) on the filter paper and record it.

Label the filter papers according to the position where they will be applied to the pad after it has been dosed with synthetic menstrual fluid.

Priming and Calibration of the Peristaltic Pump

Step 1: The pump is calibrated to deliver 20 mLs of synthetic menstrual fluid over 60 minutes. If samples are very small, a smaller dose of 10 mLs over 30 minutes can also be used. A second pump is also set up for an even smaller dose of 5 mLs over 30 minutes.

Verify operation and calibration of the peristaltic pump by filling a 250 ml beaker with approximately 50 mLs of synthetic menstrual fluid.

Step 2: Pre-weigh three separate 250 ml beakers and labeled them A, B and Cs.

Step 3: Record the weight of each beaker as a TARE weight.

Step 4: Place the three inlet (also labeled A, B and C) ends of the tubes into the menstrual fluid.

Step 5: Place the outlet ends into an empty 250 ml beaker.

Step 6: To prime the pump, turn it on and allow it to run long enough to rinse out DI water or air trapped in the lines from previous testing or sitting for long periods of time.

Step 7: Once the pump is primed and the tubes are full of synthetic menstrual fluid, confirm there's at least 40 mLs of fluid left in the main 250 ml beaker to use for calibration and testing.

Step 8: Carefully remove each tube and place the each one of the outlet tube ends into the three correspondingly labeled pre-weighed beakers. (Tube A into Beaker A, Tube B into Beaker B etc.)

Step 9: Set the timer for three minutes and start the pump to run—you will see a small amount of the fluid enter into each beaker.

Step 10: When the timer stops, carefully remove the tubes from the beakers and weigh each beaker recording the weights as Gross Weight, g.

Step 11: Subtract Tare weights from Gross weights to calculate Net weights of each individual line and record the Net Weight to confirm calibration.

All three tubes need to be confirmed as calibrated prior to testing. If there are discrepancies in the Net value of any tube greater than 10%, run the calibration again following the same steps mentioned above.

Step 12: If all three tubes are accurately calibrated, thread them through corresponding stainless steel tube holders in preparation for testing

Rewet and Liquid Distribution Test

Step 1: Weigh and record the weight of each pad.

Step 2: Place the pads to be tested on the counter inside the feminine hygiene test cabinet, and position them so the dosing tube is 1 cm above the marked insult point of the pad. If edges of pad curl, tape the pads to the countertop using lab tape so they lay flat.

Step 3: Set the lab timer for 1 hour and start the pump.

Step 4: Close the feminine hygiene test cabinet.

Step 5: At the end of the one hour dosing application, allow samples to rest for 20 minutes.

Step 6: At the end of the 20 minute rest period, position filter paper stacks on top of the corresponding sections of samples by starting in the middle, then placing the other two stacks at the front and back of the pad so they touch the middle stack.

Step 7: Set individual timers for five minutes.

Step 8: Place a rectangular weight on top of the filter papers and pads and start the 5 minute timer.

Step 9: At the end of 5 minutes, remove weight.

Step 10: Weigh filter paper stacks and record the wet weight for each one.

Step 11: Weigh the entire wet pad and record the weight.

Step 12: Cut each sample one at a time along the lines (drawn) on the pads being as precise as possible.

Step 13: Weigh each wet pad section (#1F, #1M and #1B) and record weight. Repeat this process with all other replicates until testing is complete.

Calculations:

Rewet Value—The amount of liquid absorbed by filter papers after dosing. Rewet, g=wet filter papers, g minus dry filter paper, g.

Liquid Distribution—The total amount of liquid absorbed by each (cut) section of a pad: Front, Middle and Back. Liquid Distribution, g=weight of each section+rewet value of each section minus the average dry product weight of each (tare) section.

Step 14: Failure occurs if there is run off from the pad onto the countertop. It is acceptable if run-off goes into the wings and/or side channels.

Step 15: After testing is finished, flush all peristaltic pump lines with DI water

Step 16: Store remaining synthetic menstrual fluid in refrigerator

Intake and Rewet for Feminine Hygiene Product

Preparation of SMF from Processed Swine Blood (PSB)

Step 1: Processed swine blood arrives in bags.

Step 2: Gently knead the bag to mix the contents.

Step 3: Cut off an edge of the bag and pour the contents into a 250 ml plastic bottle.

Step 4: Remove the label from the bag and place it on the 250 ml plastic bottle.

Step 5: From the 250 ml plastic bottle, fill a 50-ml test tube approximately 40-ml. Place the excess PSB back into the fridge. Since the viscosity of the blood can change over time, it is important to remove small quantities at a time.

Step 6: Allow the PSB to come to room temperature for approximately 1 hour.

Step 7: Take the viscosity reading of the PSB. Viscosity can change between bags and overtime within each bag. The viscosity of the blood can change the intake time.

Step 8: While testing, a second 50-ml test tube can be filled with PSB to allow to come to temperature.

Feminine Hygiene Product Sample Preparation

Step 1: A prototype should contain a backsheet and the pad. Measure product absorbent core length and width.

Step: 2 Mark center of absorbent core length and width with an “X”. The “X” will be the insult point.

Step 3: For commercial products that have wings, the center should be the middle of the wings.

Intake and Rewet Testing Procedure for Feminine Hygiene Products:

Step 1: Place protruding side of the insult block on the product so the hole is centered over the insult point.

Step 2: Weigh three filter paper for the “Before rewet” weight and record their initial weight.

Step 3: Prime the syringe with menstrual fluid simulant.

Step 4: Draw 2 mL of PSB into the syringe, ensuring there are no air bubbles.

Step 5: Place the tip of the syringe over the insult point on the product, resting the syringe on the insult block, but not adding any pressure onto the block.

Step 6: Start dispensing the menstrual fluid simulant, targeting a dose rate of 5 mL/3 seconds. Simultaneous, start the stopwatch and 10 minute timer.

Step 7: Stop stopwatch when fluid is absorbed.

Step 8: When the 10 minutes timer ends, repeat steps 3-6 two more times.

Step 9: After the third round of the 10 minutes timer ending, remove the insult block.

Step 10: Place three rewet filter paper on top of the product, covering majority of the fluid.

Step 11: Place the weight on top of the filter paper. Start a timer for 2 minute.

Step 12: When the 2 minute timer ends, remove the weight and filter paper from the product.

Step 13: Weigh the filter paper for the “After rewet” weight and record the weight.

Step 14: If wicking distance is desired, measure the length and width of the PSB on the sample.

Step 15: Record the length and width of fluid distance wicked.

Step 16: Fold the product and dispose of it in a biohazard waste bucket.

Step 17: Clean the acrylic intake rewet board with disinfectant solution.

### EXAMPLES

### Example 1. Fabrication of Composite Fabrics

The crosslinked fiber layer of the present Example was fabricated by using lab scale air-laying equipment. Crosslinked fibers in dry loose fluff form were fed into a chamber with blunt blends blades to disperse the fibers further. Air was supplied to the chamber to push crosslinked fibers through a wire mesh onto a tissue laid on a 14 in×14 in forming wire. The air-laid crosslinked fiber mat was then sandwiched between blotter papers and pressed at 12000 psi. Pressed mats were cut to dimensions of 12 in×12 in, then stored for later use. Resin bonded carded web and spunbond materials were prepared by cutting the nonwovens to the same dimensions as the crosslinked fiber mat, 12 in×12 in. Staple fibers were prepared by putting the loose, dry staple fibers into 2 L of water and subjecting the mixture to 1500 rpm in a British Disintegrator to disperse the staple fibers. A 12 in×12 in lab scale wet-laying piece of equipment was prepared by placing a forming wire over the drainage area and sealing the equipment such that water did not leak out. The staple fiber-water slurry was mixed with low velocity air impingement for 2 minutes. After 2 minutes, air impingement was stopped and the water was drained, depositing staple fibers onto the forming wire. The wet-laid staple fiber mat was sandwiched between blotter papers and dried at 105° C. for 15 minutes.

To prepare the two layers of crosslinked cellulose fiber and nonwoven/staple fiber for hydroentanglement, the air-laid crosslinked cellulose fiber mat was removed from blotters and placed onto either a resin bonded carded web, a carded web, spunbond, or wet-laid staple fiber mat such that the crosslinked cellulose fiber mat was immediately positioned on the nonwoven/staple fiber layer.

Hydroentanglement of samples was performed with lab scale hydroentanglement equipment including of a conveyor belt, forming wire on top of the conveyor belt, jet strip positioned over the conveyor belt to extrude water jets, and a pump to control the pressure of water jets coming out of the jet strip. The forming wire was positioned over the conveyor belt such that it was not under the jet strip. The combined mat of crosslinked fiber and nonwoven/staple fiber was placed onto the forming wire such that it was not directly under the jet strip and the crosslinked layer was directly facing the jet strip while the nonwoven/staple fiber layer was directly contacting the forming wire. The water pump was turned on to provide water jets at a low pressure, below 100 psi. One pass was defined as the material to be hydroentangled being moved through the water jets in one direction from one end to the opposing end without stopping or changing direction of the conveyor belt. The conveyor belt was manipulated to subject the crosslinked fiber and nonwoven/staple fiber mat to four passes at the low pressure condition to pre-wet the fibers. The pressure of the water jets was then manipulated to achieve pressures listed in Table 2 and the crosslinked fiber and nonwoven/staple fiber mat is subjected to one pass at that pressure. E.g., hydroentanglement of sample 10, a crosslinked fiber mat on top of a resin bonded carded web, consisted of 4 pre-wetting passes followed by one pass at 200 psi. Once samples were hydroentangled, they were restrained between two Teflon mats and dried in an over at 105° C. for 15-20 minutes.

Table 2 shows different combinations of crosslinked fiber and nonwoven material hydro-entangled at varying pressures. As the hydro-entanglement pressure increased, the degree of crosslinked cellulose fiber penetration into the nonwoven increased. In Table 2, the nonwoven was either resin bonded carded web: A web included of synthetic fibers that have been bound by a resin; a spunbond web formed of filaments from a melt process; or staple fibers, which are synthetic fibers laid down as a mat and not bonded by any mechanism.

TABLE 2
Composition of composite fabrics and hydro-entanglement
pressures.

| | | | Nonwoven | |
|---|---|---|---|---|
| | Crosslinked | | Basis | Hydro- |
| | Fiber Basis | Nonwoven | Weight | entanglement |
| Name | Weight (g/m²) | Type | (g/m²) | Pressure (psi) |
| Sample 10 | 110 | Resin bonded | 40 | 200 |
| | | carded web | | |
| Sample 11 | 110 | Resin bonded | 40 | 600 |
| | | carded web | | |
| Sample 12 | 110 | Resin bonded | 40 | 1000 |
| | | carded web | | |
| Sample 14 | 40 | Spunbond | 15 | 200 |
| Sample 15 | 40 | Spunbond | 15 | 400 |
| Sample 18 | 110 | Staple fiber | 40 | 200 |
| | | (unbonded) | | |
| Sample 19 | 110 | Staple fiber | 40 | 600 |
| | | (unbonded) | | |

### Example 2. Diaper Constructs and Properties

Referring to Table 3, various BCWs can be combined with a crosslinked cellulose fiber to produce a range of densities for the resulting composite structures. Despite the difference in densities, all composite fabrics including BCW and crosslinked cellulose fiber showed improved rewet and intake values.

TABLE 3
Compositions of composite fabrics.

| | Basis Weight | | |
|---|---|---|---|
| Material | (g/m²) | Caliper (mm) | Density (g/cm³) |
| TABCW/110 | 159 | 1.48 | 0.099 |
| Helix ™ Air ®+ | | | |
| RBCW/110 Helix ™ | 146 | 1.53 | 0.095 |
| Air ®+ | | | |
| RBCW/110 Helix ™ | 143 | 2.76 | 0.052 |
| Air ®+ | | | |

Two diaper constructs were formed for this Example, referred to as ADL and core-wrap constructs. The base diaper for the constructs was Commercial Diaper 1, a diaper with a nonwoven acquisition layer, a crosslinked cellulose fiber under the nonwoven, and a fluffless core with channels. Commercial Diaper 2 has a multi-layer core design and was used as a comparison for core-wrap diaper constructs using a composite fabric of the present disclosure.

For the ADL construct, the nonwoven and crosslinked cellulose fiber were removed and the replacement material was cut to the dimensions of the nonwoven layer.

For the core-wrap construct, the nonwoven and Helix™ fiber were removed. The core was removed and wrapped by either a composite fabric material.

Example 2 shows that nonwoven used in the composite structure can be through-air bonded or resin bonded. Example 2 also shows the magnitude of improvement in absorbent properties are unique to using crosslinked fiber as the cellulosic fiber layer. The nonwoven can range from 7700-18500 IPRP flow rate and maintain performance when utilized in the crosslinked fiber containing composite. Composites with a basis weight of 150 gsm±10% can range in density from 0.052-0.099 g/cm³ and have no change in diaper construct performance.

### Example 3. Diaper Constructs and Properties

Referring to Table 4, a series of TABCW and crosslinked cellulose fiber composite fabrics were made.

Material Attributes—Basis Weight, Caliper, Density

At roughly the same basis weight and hydro-entanglement conditions, Helix™ as the fiber component increases the caliper of the composite by ˜14%. Using the Groz-B jet strip increases the caliper of the composite by ˜14%.

TABLE 4
Material attributes.

| | Basis Weight | | Density |
|---|---|---|---|
| Material | (g/m²) | Caliper (mm) | (g/cm³) |
| TABCW/Helix ™ @ 110 | 153 | 1.57 | 0.098 |
| gsm | | | |
| TABCW/Helix ™ Air ®+ @ | 150 | 1.38 | 0.109 |
| 110 gsm | | | |
| TABCW/Helix ™ Air ®+ @ | 150 | 1.58 | 0.095 |
| 110 gsm with Groz-B 64 | | | |
| NW/Helix ™ Air ®+ 50 gsm | 90 | 1.02 | 0.15 |
| NW/Helix ™ Air ®+ 110 gsm | 150 | 0.65 | 0.13 |

TABCW is a through-air bonded carded web that serves as the nonwoven portion of the composite.

Two diaper constructs were formed for this experiment, referred to as ADL and core-wrap constructs. The base diaper for the constructs is Commercial Diaper 1, a fluffless core diaper with a nonwoven acquisition layer and a Helix™ fiber distribution layer under the nonwoven. Commercial Diaper 2 has a multi-layer core design and was used as a comparison for core-wrap diaper constructs using a composite fabric of the present disclosure.

For the ADL construct, the nonwoven and Helix™ fiber distribution layer were removed and the replacement material was cut to the dimensions of the nonwoven layer.

For the core-wrap construct, the nonwoven and Helix™ fiber distribution layer are also removed. The core is removed and wrapped by either a composite material of the current disclosure.

Diapers Constructed:

TABCW/Helix™ 110 gsm core-wrap

TABCW/Helix™ 110 gsm ADL

TABCW/Helix™ Air®+110 gsm ADL

TABCW/Helix™ Air®+110 gsm core-wrap, Groz-B 64 jet strip

NW/Helix™ Air®+50 gsm core-wrap

The no load saddle wicking test and flat acquisition under load test are as described in test method section.

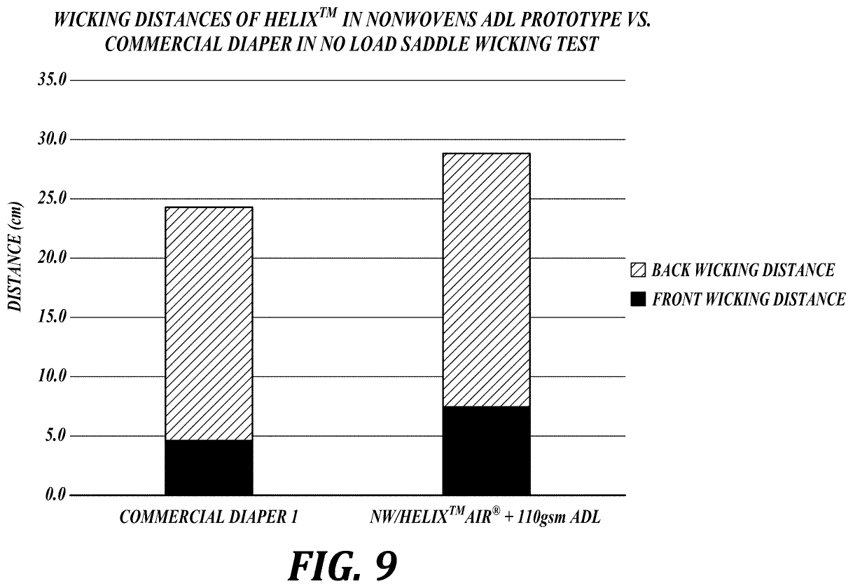

FIG. 9 is a bar graph showing a comparison of wicking distance from insult point of a composite fabric of the present disclosure in ADL diaper constructs in a no load saddle wicking test. Statistically, the deconstructed control diaper wicked less distance towards the back. Both the Helix™ (not shown) and Helix™ Air®+ composite fabrics wicked more distance than the control. The Helix™ composite fabric was able to wick further towards the front than the Helix™ Air®+ composite fabric. Increased wicking distance indicates better utilization of the core.

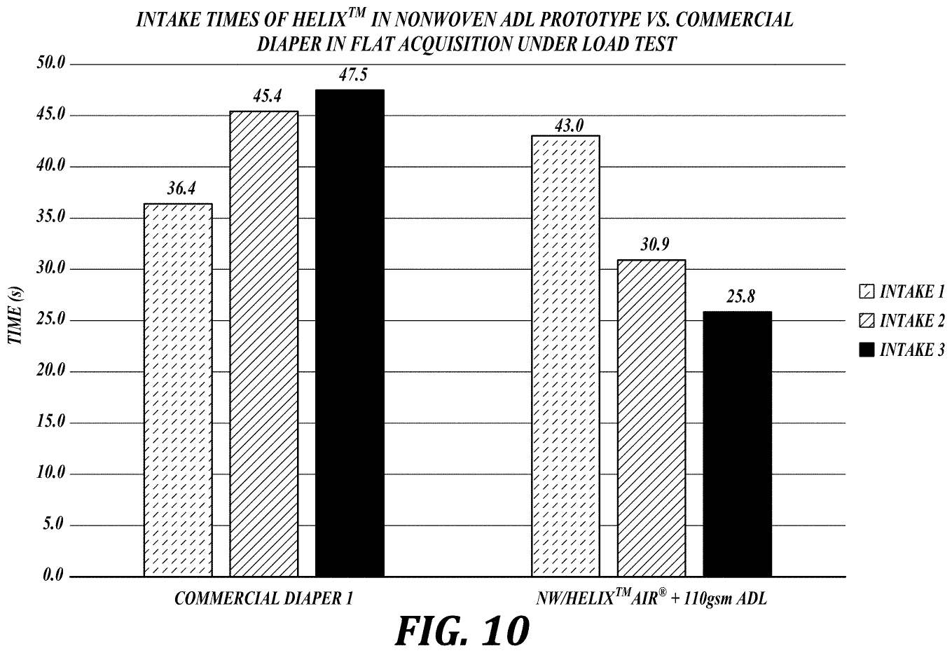

FIG. 10 is a bar graph showing a comparison of a composite fabric of the present disclosure in ADL diaper constructs with respect to intake times for the flat acquisition under load test. Deconstructing and reconstructing the control diaper has no significant impact on the intake time. Crosslinked fiber including composites had significantly lower times for intakes 2 and 3. There was no significant difference in intake times when Helix™ (not shown) and Helix™ Air®+ were used as the fiber component of the composite structure in the diaper constructs.

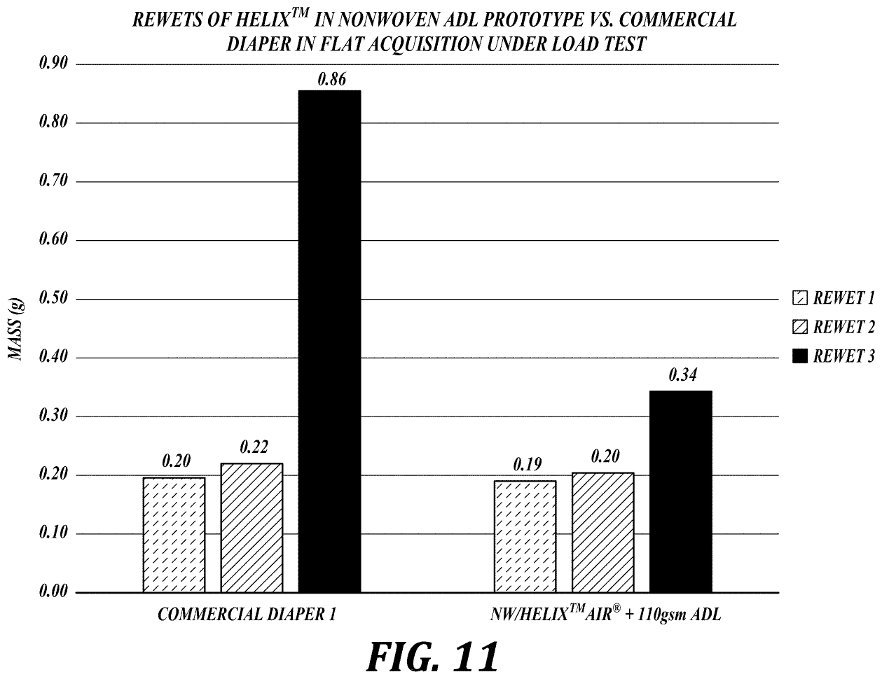

FIG. 11 is a bar graph showing a comparison of a composite fabric of the present disclosure in ADL diaper constructs with respect to rewet values for the flat acquisition under load test. Decreased rewet values were shown for intakes 1, 2, and 3, with the rewet value for intake 3 being dramatically smaller than in Commercial Diaper 1.

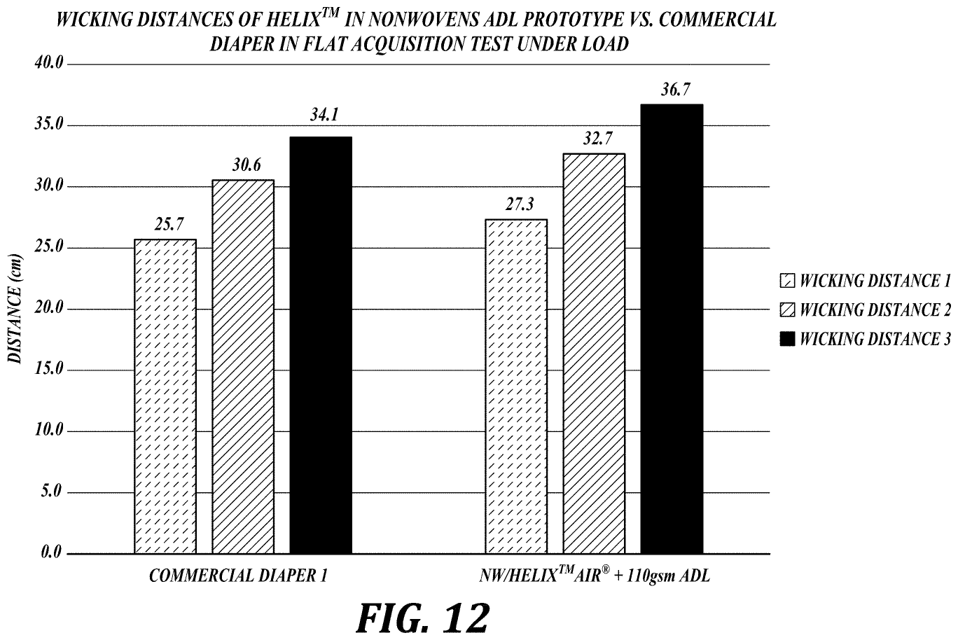

FIG. 12 is a bar graph showing a comparison of average wicking distances of the diaper for a composite fabric of the present disclosure in ADL diaper constructs, as compared to Commercial Diaper 1. The composite fabric in ADL diaper constructs wicked significantly further than the control diaper. Helix™ (not shown) as the crosslinked fiber component of the current disclosure wicks further in intake 1 than the Helix™ Air®+ version. However, the wicking distance from doses 2 and 3 are not statistically different between the two crosslinked fiber constructs.

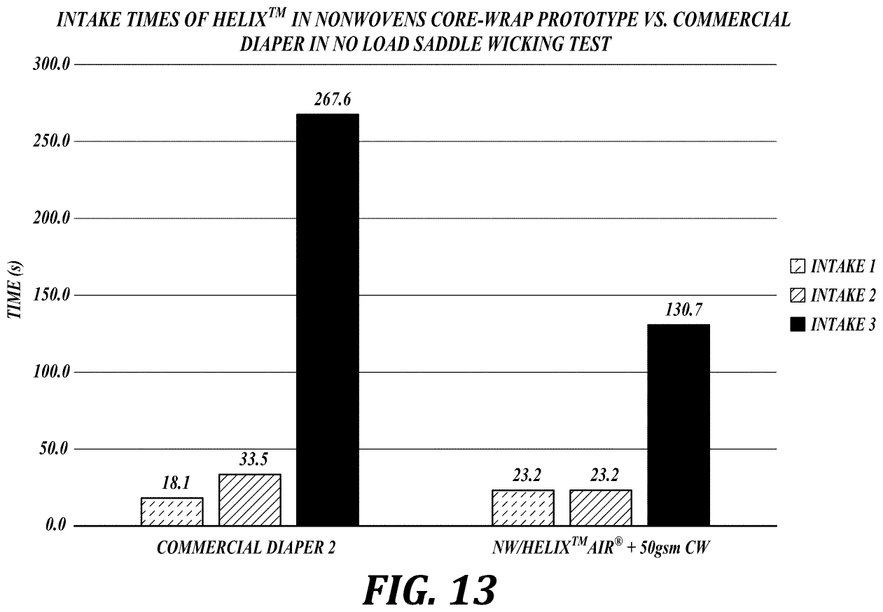

FIG. 13 is a bar graph showing a comparison of average intake times of diapers including composite fabrics of the present disclosure in core-wrap diaper constructs in a no load saddle wicking test. All diapers including composite fabrics of the present disclosure had significantly improved intake times versus the control Commercial Diaper 2. There was no significant difference between intake times of diapers including Helix™ (not shown) or Helix™ Air®+ containing composites. Helix™ Air®+ as the fiber component in the composite shows no significant difference when hydro-entangled with different jet strips.

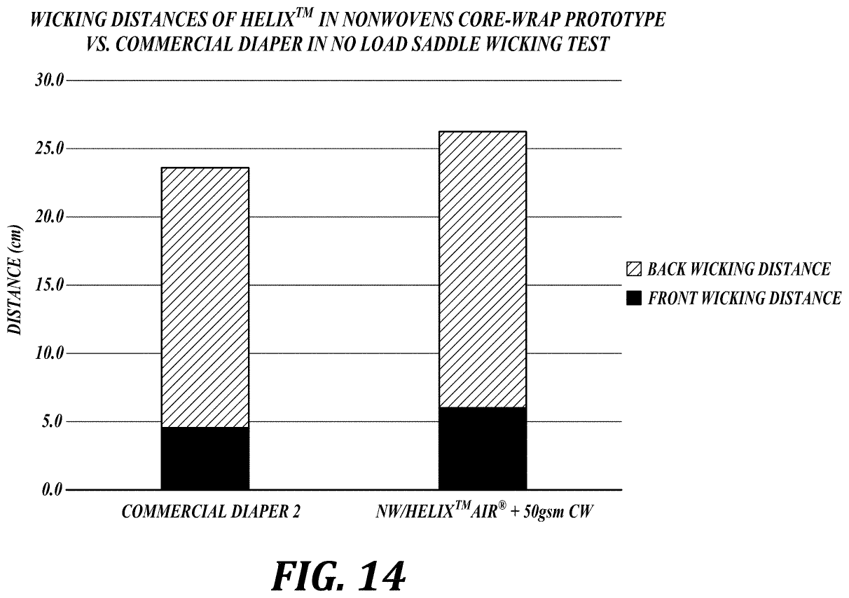

FIG. 14 is a bar graph showing a comparison of wicking distance from insult point of a composite fabric of the present disclosure in core-wrap diaper constructs in a no load saddle wicking test. All diaper constructs including the composite fabrics of the present disclosure showed significantly improved wicking distances versus the control. Helix™ (not shown) as the fiber component in the composite shows improved wicking distance versus the Helix™ Air®+ as the fiber component.

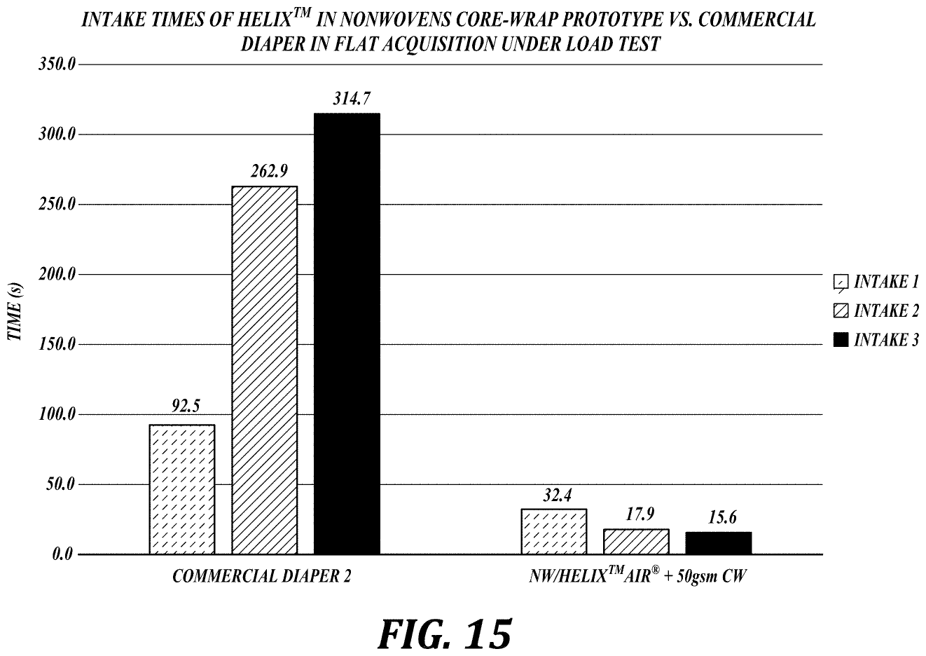

FIG. 15 is a bar graph showing a comparison of a composite fabric of the present disclosure in core-wrap diaper constructs with respect to intake times from the flat acquisition under load test.

All diaper constructs including the composite fabric of the present disclosure showed significant improvement in intake time versus the control.

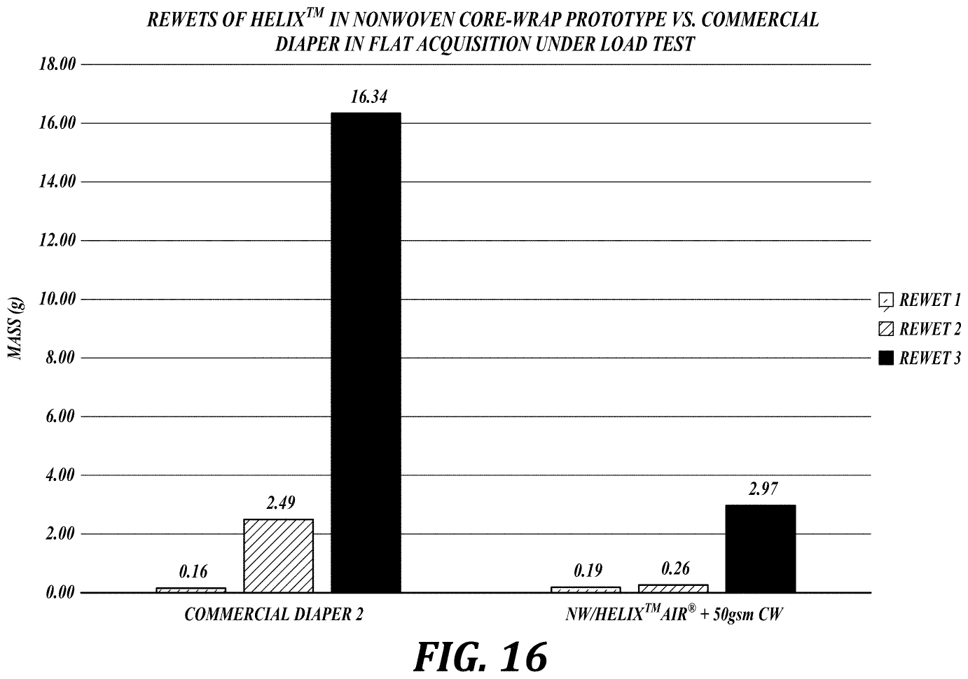

FIG. 16 is a bar graph showing a comparison of a composite fabric of the present disclosure in core-wrap diaper constructs with respect to rewet values from the flat acquisition under load test. All diaper constructs including the composite fabrics of the present disclosure showed significant improvement over the control diaper.

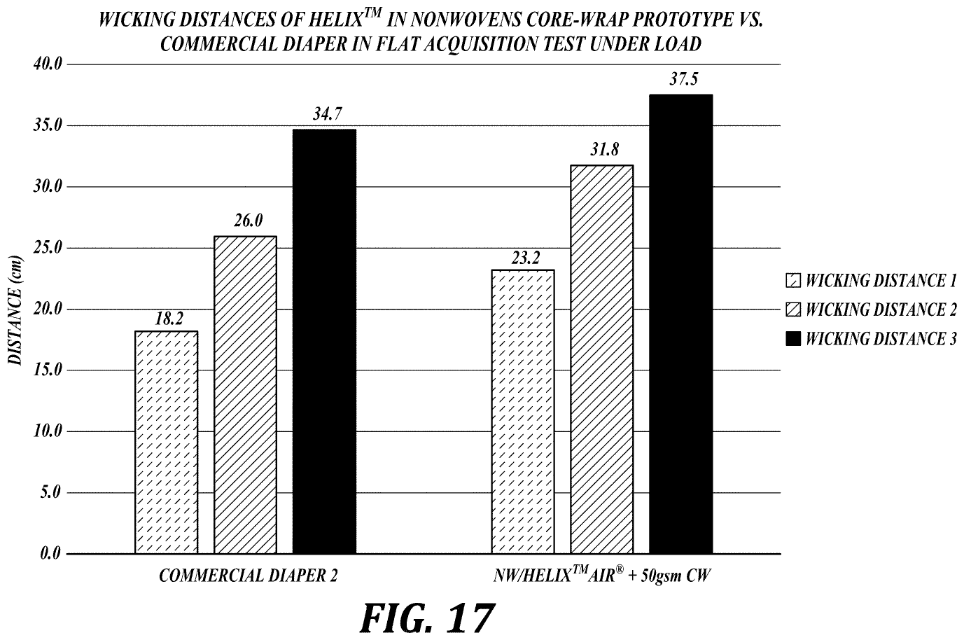

FIG. 17 is a bar graph showing a comparison of average wicking distances of a composite fabric of the present disclosure used in a core-wrap diaper design. The diaper construct including the core-wrap was a more simplified design compared to the Commercial Diaper 2's multi-layer core design. All diaper constructs employing the composite fabrics of the present disclosure showed improved wicking distance towards the front for doses 1 and 2. When Helix™ is used as the crosslinked cellulose fiber component in the composite fabric, the test fluid immediately wicked the full distance of the core of the diaper. All crosslinked fiber composite containing diaper constructs showed significantly improved wicking distances versus the control diaper.

Example 3 showed that there was no significant difference in diaper construct performance when entangling Helix™ Air®+ with a different patterned jet strip. Composites with Helix™ exhibit improved wicking versus Helix™ Air®+ in all diaper constructs. Improved wicking occurs through all insults or the first two insults.

TABLE 5
ADL Application - Flat Acquisition Under Load

| | | 150 gsm | Average |
|---|---|---|---|
| | Commercial | NW/Helix ™ | Commercial Fluff |
| | Diaper 1 | Air ®+ | Core Products |
| l^{st} Intake (s) | 36.4 | 43.0 | 46.8 |
| 2^{nd} Intake (s) | 45.4 | 30.9 | 84.1 |
| 3^{rd} Intake (s) | 47.5 | 25.8 | 98.8 |
| l^{st} Rewet (g) | 0.20 | 0.19 | 0.16 |
| 2^{nd} Rewet (g) | 0.22 | 0.20 | 0.38 |
| 3^{rd} Rewet (g) | 0.86 | 0.34 | 6.07 |
| 1^{st} Wicking | 25.7 | 27.3 | 21.6 |
| Distance (cm) | | | |
| 2^{nd} Wicking | 30.6 | 32.7 | 27.4 |
| Distance (cm) | | | |
| 3^{rd} Wicking | 34.1 | 36.7 | 33.1 |
| Distance (cm) | | | |

TABLE 6
Core-wrap application - Flat Acquisition under Load

| | Commercial | 90 gsm | Average |
|---|---|---|---|
| | | NW/Helix ™ | Commercial Fluffless |
| | Diaper 2 | Air ®+ | Core Products |
| 1^{st} Intake (s) | 92.5 | 32.4 | 86.3 |
| 2^{nd} Intake (s) | 262.9 | 17.9 | 264.5 |
| 3^{rd} Intake (s) | 314.7 | 15.6 | 318.6 |
| 1^{st} Rewet (g) | 0.16 | 0.19 | 0.15 |
| 2^{nd} Rewet (g) | 2.49 | 0.26 | 2.85 |
| 3^{rd} Rewet (g) | 16.34 | 2.97 | 12.39 |
| 1^{st} Wicking | 18.2 | 23.2 | 20.8 |
| Distance (cm) | | | |
| 2^{nd} Wicking | 26.0 | 31.8 | 27.6 |
| Distance (cm) | | | |
| 3^{rd} Wicking | 34.7 | 37.5 | 33.2 |
| Distance (cm) | | | |

### Example 4. Lab Carded Staple Fiber Composites

TABLE 7
Intake Times of Lab Carded Staple Fiber Composites in Flat
Acquisition Under Load test

| Sample | Intake 1 (s) | Intake 2 (s) | Intake 3 (s) |
|---|---|---|---|
| Commercial | 61.0 | 198.4 | 217.6 |
| Diaper 2 | | | |
| NW/Helix ™ | 32.4 | 17.9 | 15.6 |
| Air ®+ 50 gsm | | | |
| Staple Fiber | 36.0 | 26.6 | 18.1 |
| Rayon Fiber | 48.8 | 32.8 | 22.4 |

TABLE 8
Rewet Values of Lab Carded Staple Fiber Composites in Flat
Acquisition Under Load test.

| Sample | Rewet 1 (g) | Rewet 2 (g) | Rewet 3 (g) |
|---|---|---|---|
| Commercial | 0.13 | 0.68 | 9.38 |
| Diaper 2 | | | |
| NW/Helix ™ | 0.19 | 0.26 | 2.97 |
| Air ®+ 50 gsm | | | |
| Staple Fiber | 0.21 | 0.41 | 3.50 |
| Rayon Fiber | 0.16 | 0.44 | 2.90 |

TABLE 9
Wicking Distances of Lab Carded Staple Fiber Composites in Flat
Acquisition Under Load test

| | Wicking | Wicking | Wicking |
|---|---|---|---|
| | Distance 1 | Distance 2 | Distance 3 |
| Sample | (cm) | (cm) | (cm) |
| Commercial | 21.1 | 27.7 | 32.3 |
| Diaper 2 | | | |
| NW/Helix ™ | 23.2 | 31.8 | 37.5 |
| Air ®+ 50 gsm | | | |
| Staple Fiber | 24.4 | 34.2 | 38.0 |
| Rayon Fiber | 27.4 | 36.9 | 38.3 |

The above three tables shows when the nonwoven layer was comprised of unbonded staple fibers, formed by the carding process and followed by subsequent hydroentanglement with Helix™ Air®+ fibers, the resulting composite still performed comparably to the composite when formed with a pre-bonded nonwoven web. Both petroleum-based staple fibers and cellulose derived staple fibers were used as the nonwoven layer in this Example. Composites made with the carded staple fibers were made into core-wrap prototypes following the same procedure described in Example 2. When compared with the composite made with a pre-bonded nonwoven web, the carded web composites exhibit a similar intake time trend in the Flat Acquisition Under Load test. Additionally, both the rewet values and wicking distances of the carded web composites are within value ranges previously measured with the pre-bonded nonwoven composites. The variety of staple fibers that can be used in the nonwoven portion of the composite allows for flexibility in sourcing of raw materials for manufacturing of the composite.

### Example 5. Fluffless Core and Fluff Core Diaper Comparisons

The present Example shows that the benefit offered by the hydroentangled crosslinked fiber and nonwoven composite fabrics of the present disclosure for the core-wrap application (see, e.g., FIG. 4). Further benefits can be observed if the basis weight of crosslinked fiber is increased.

As an ADL, the crosslinked fiber composite reaches parity in saddle wicking results. The crosslinked fiber composite stands out in flat acquisition under load, improving intake times, rewet values, and early wicking distances. It is possible to make multiple grades of material by varying the basis weight.

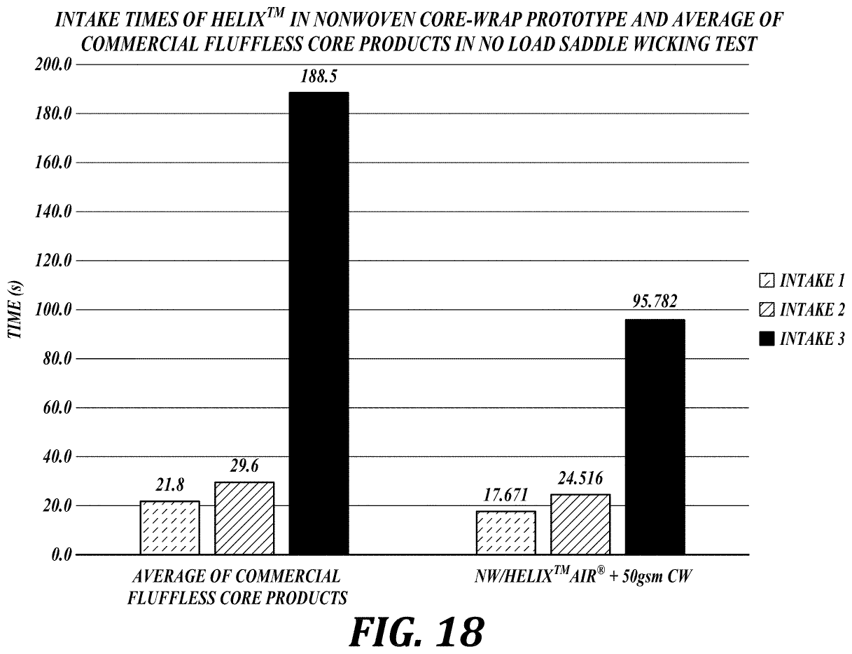

FIG. 18 is a bar graph showing average intake times of fluffless diapers in a no load saddle wicking test for a diaper using the composite fabric in a core-wrap configuration compared to averages of commercial fluffless core diapers. The composite fabric was able to significantly improve intake time of fluid in the core-wrap application for the no load saddle wicking test.

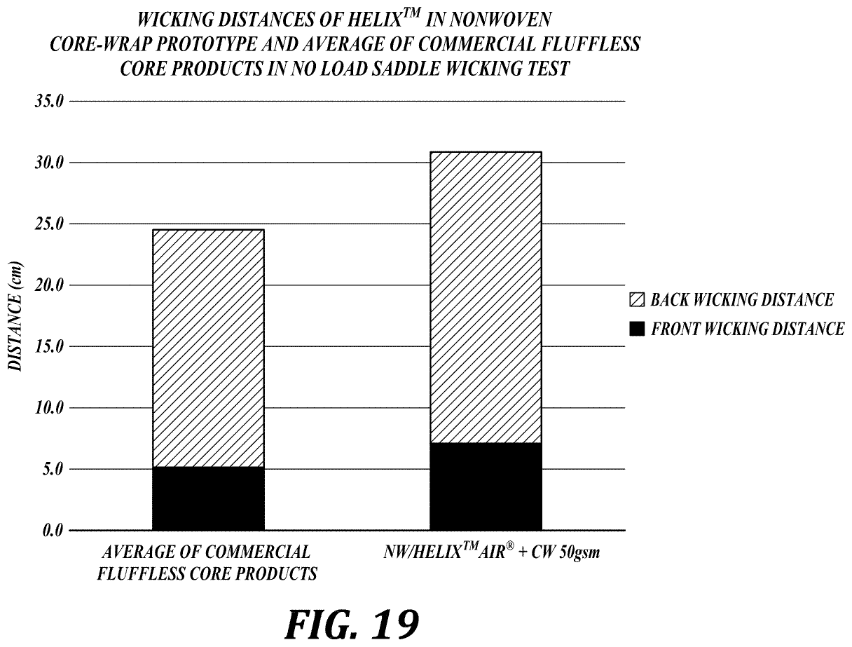

FIG. 19 is a bar graph showing a comparison of wicking distances from insult point for a diaper using the composite fabric in a core-wrap configuration compared to averages of commercial fluffless core diapers. The composite fabric was able to increase wicking distances compared to the average wicking distance of commercial fluffless core diapers.

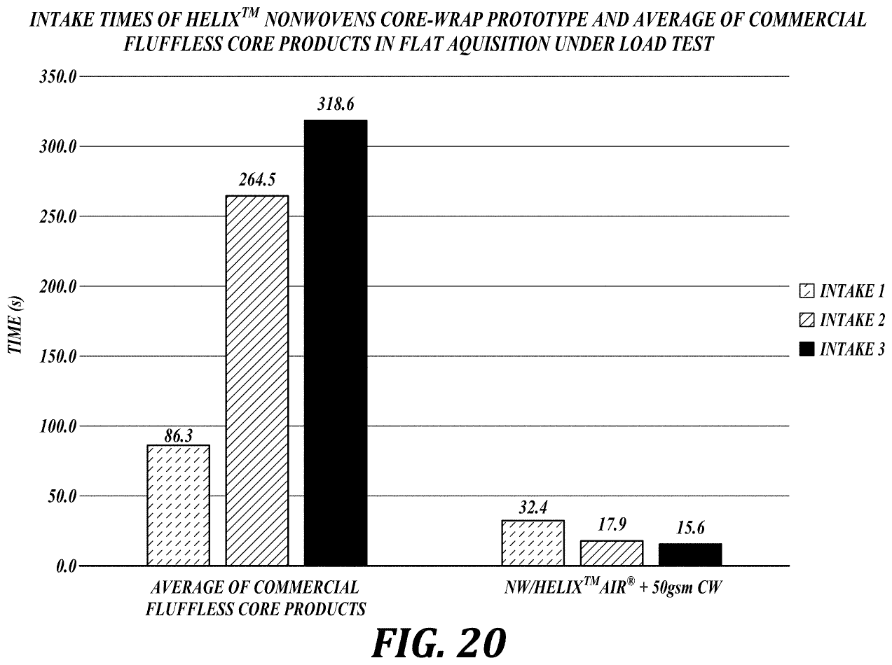

FIG. 20 is a bar graph showing a comparison of fluffless diaper intake times in a flat acquisition under load test for a diaper using the composite fabric in a core-wrap configuration compared to averages of commercial fluffless core diapers. The composite fabric was able to significantly improve the intake time for all three fluid insults in the core-wrap application.

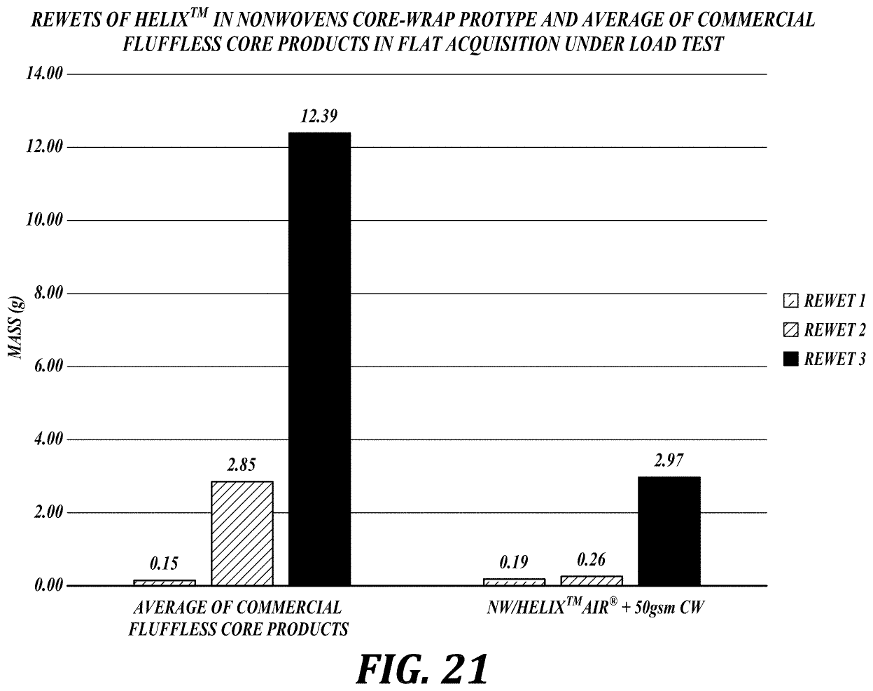

FIG. 21 is a bar graph showing a comparison of fluffless diaper rewet values in a flat acquisition under load test for a diaper using the composite fabric in a core-wrap configuration compared to averages of commercial fluffless core diapers. The composite fabric was able to significantly improve the second and third rewet values in the core-wrap application.

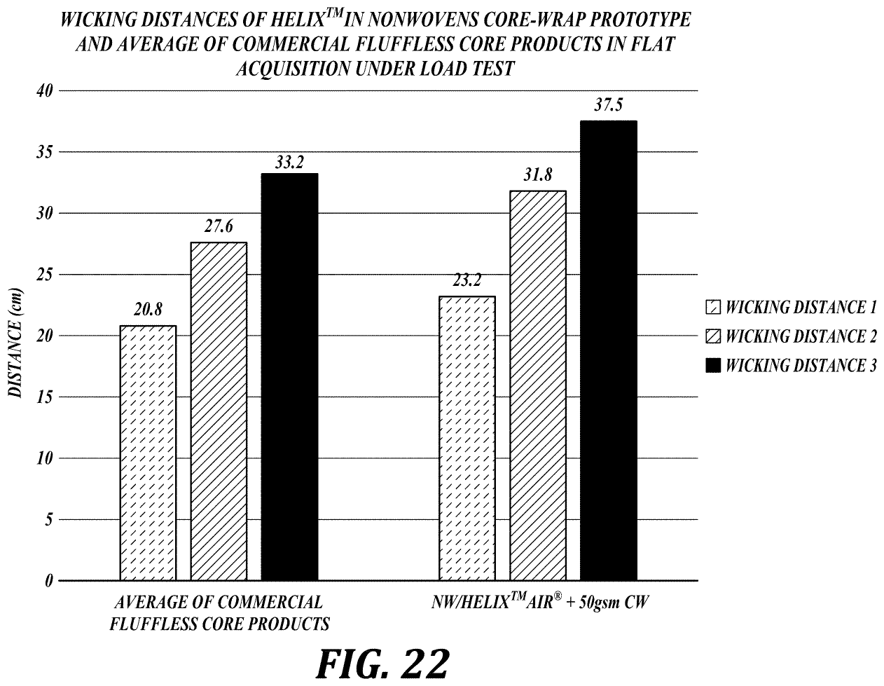

FIG. 22 is a bar graph showing a comparison of average wicking distances of fluffless diapers in a flat acquisition under load test for a diaper using the composite fabric in a core-wrap configuration compared to averages of commercial fluffless core diapers. The composite fabric was able to increase wicking distances for all three fluid insults in the flat acquisition under load test in the core-wrap application.

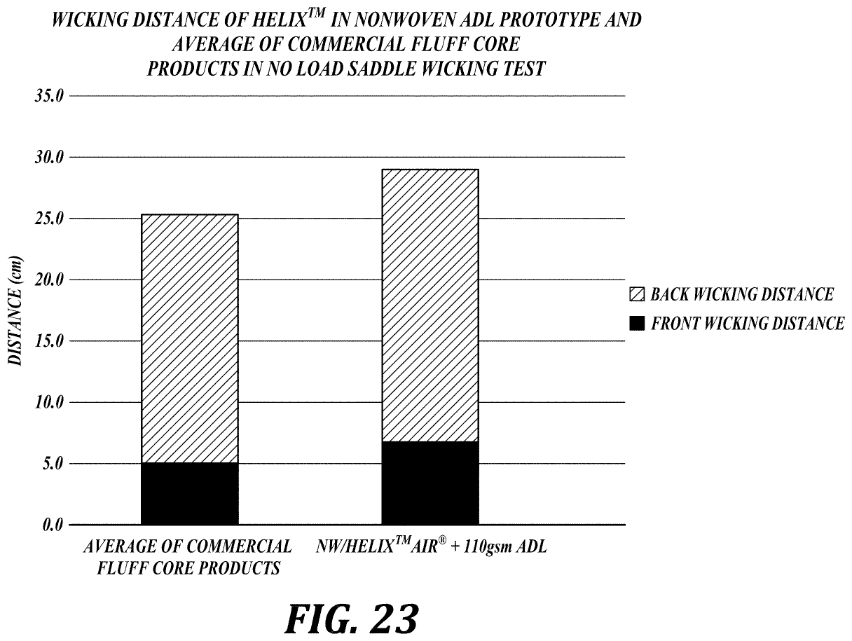

FIG. 23 is a bar graph showing a comparison of fluff core diapers from insult point of diaper constructs in a no load saddle wicking test for a diaper using the composite fabric in an ADL configuration compared to averages of commercial fluff core diapers. The composite fabric was able to increase wicking distance against the average wicking distance of commercial fluff core diapers in the no load saddle wicking test.

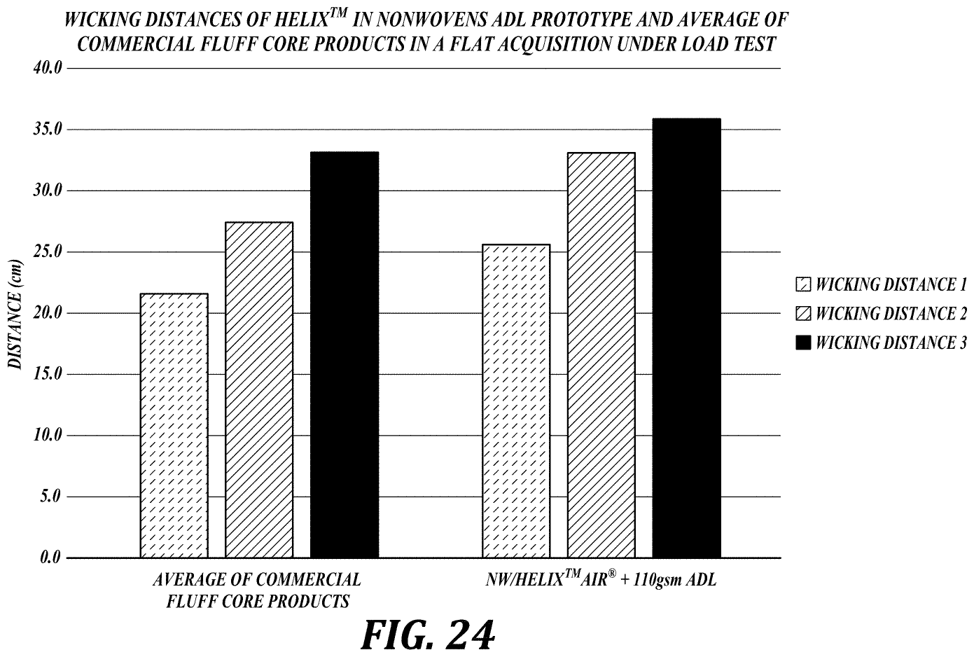

FIG. 24 is a bar graph showing a comparison of wicking distances of a diaper using the composite fabric in an ADL configuration compared to averages of commercial fluff core diapers. The composite fabric was able to significantly increase wicking distances against the average wicking distances of commercial fluff core diapers in the flat acquisition under load test, for all three fluid insults.

### Example 6. Diaper and Adult Incontinence Product (Wet-Laid Composite)—Constructs and Properties

Described below is a pilot approximation of commercially available hybrid carded pulp technology. Production of the Helix™ in nonwovens composite on a wet-laid pilot line began with fiber stock preparation. Dry Helix™ Air®+ fibers were added to a stock tank of water and diluted to a concentration of 22%. The stock tank was constantly stirred with an agitator that did not damage the quality of the fibers. The stock was pumped from the stock tank to the headbox of the wet-laying system. Along the way, the stock was further diluted with water to improve formation of the fibers as they were deposited onto the forming wire. The diluted stock then entered the headbox and was distributed onto the forming wire to form a web of Helix™ Air®+ fibers. Water was then drained from the web from either gravity or vacuum slits below the forming wire. When the web was sufficiently dry, it was transferred from the forming wire onto a pre-bonded nonwoven web. The nonwoven web width was equivalent or greater in width as compared to the Helix™ Air®+ web. The bi-layered nonwoven and fiber web were pre-hydroentangled with low pressure water jets to help keep the two layers together. The water jets first came in contact with the fibrous side of the web to push the fibers into the nonwoven. After pre-hydroentanglement, water was removed via vacuum slits. The web was then threaded through a heated can dryer system where minimal heat was applied to help dewater the web to approximately 50% solids content. The partially-dried web was then wound into a roll and wrapped in plastic to prevent further moisture loss. The plastic wrapped rolls were then saved for further hydroentanglement.

Rolls were loaded onto an unwind stand and unwound such that the nonwoven side of the web contacted the carrier web and the fiber side of the web was faced towards the hydroentanglement jet heads. The carrier web brought the unbonded Helix™ in nonwovens material through at least two hydroentanglement jet heads to further push the Helix™ Air®+ fibers into the nonwoven, bonding the two layers together. The composite structure was dewatered via vacuum slits and passed through a through-air drying system to completely dry the composite to greater than 90% solids content. The dry composite was wound into a roll for further use.

While a 2-step process for making the composite fabric is described in the present Example, a person of skill in the art would understand that a 1-step process can be readily carried out.

Referring to Table 10, the composite material used in this Example was formed of a fiber layer composed of 100% Helix™ Air®+ and the nonwoven layer was a through air bonded carded web. Sample Codes 1-4 were tested for their performance as an ADL; samples 5 and 6 were tested for their performance as a core-wrap.

TABLE 10
Test composite material compositions.

| Sample Code | Composite basis | Helix ™ Air ®+ | Nonwoven |
|---|---|---|---|
| (#) | weight (BW, g/m²) | BW (g/m²) | BW (g/m²) |
| 1 | 150 | 110 | 40 |
| 2 | 140 | 110 | 30 |
| 3 | 120 | 80 | 40 |
| 4 | 110 | 80 | 30 |
| 5 | 90 | 50 | 40 |
| 6 | 80 | 50 | 30 |

Commercial baby diapers and a commercial adult incontinence product were selected as commercial comparatives for prototypes. The ADL from each product was removed and replaced with composite fabric of the exact same dimensions: Codes 1-4 were tested in each commercial product.

The intake times of the Commercial Comparative Diapers in a flat acquisition under load test were obtained. Commercial Comparative Diapers 1 and 4 had the fastest intake times. In an ADL diaper construct, with Code 1, 2, 3, or 4 composite fabric samples replacing the ADL of Commercial Comparative Diapers 1, 2, 3, or 4, in a flat acquisition under load test, composite fabric sample Code 1 exhibited a noticeably reduced intake time compared to Comparative Diapers 2 or 3, respectively. A reduction in intake time compared to Comparative Diaper 1 was seen for all ADL diaper constructs using Codes 1, 2, 3, and 4 composite fabric samples. A reduction in intake time compared to Comparative Diaper 4 was seen for ADL diaper constructs using Codes 1, 3, and 4 composite fabric samples, and for intakes 1 and 3 in the case of an ADL diaper construct using a Code 2 composite fabric sample.

For the intake times of embodiments of core-wrap diaper constructs, with Code 5 or 6 composite fabric samples wrapping the absorbent core of Commercial Comparative Diaper 1, in a flat acquisition under load test, both Code 5 and 6 composite fabric samples provided a significant reduction in intake time compared to Commercial Comparative Diapers 1 and 5.

Commercial Comparative Diapers 1 and 4 have the lowest rewet values at Rewet 3 among the Commercial Comparative Diapers, in a flat acquisition under load test. Across Commercial Comparative Diapers 1-4, code 1 composite fabric offered improvement in rewet values, and this was particularly noticeable in rewet 3. An improvement in rewet values at Rewets 2 and 3 compared to Commercial Comparative Diaper 5 was also observed, in particular in core-wrap diaper constructions where a Code 5 or 6 composite fabric sample wraps the absorbent core of Commercial Comparative Diaper 1, in a flat acquisition under load test.

For the average total wicking distance in embodiments of ADL diaper constructs, using Code 1, 2, 3, or 4 composite fabric samples to replace the ADL of Commercial Comparative Diaper 1, in a flat acquisition under load test, the wicking distances were improved compared to those of Commercial Comparative Diaper 1.

For the average total wicking distance in embodiments of core-wrap diaper constructs, using Code 5 or 6 composite fabric samples to wrap the absorbent core of Commercial Comparative Diaper 1, in a flat acquisition under load test, the wicking distances were improved compared to those of Commercial Comparative Diapers 1 and 5.

The intake times for ADL constructs using Code 1, 2, 3, and 4 were improved compared to the intake times of Comparative Diaper 4, in a no load saddle wicking test.

Core-wrap diaper constructs made with Code 5 and Code 6 composite fabric samples show improvement in intake times 2 and 3 compared to Commercial Comparative Diaper 5, in a no load saddle wicking test.

In an ADL diaper construct, the wicking distances using Code 1, 2, 3, or 4 composite fabric samples were improved (i.e., greater than) to those of the Commercial Comparative Diaper 3 and Commercial Comparative Diaper 1.

In a core-wrap diaper construct, the wicking distances using Code 5 or 6 composite fabric samples to wrap the absorbent core were improved (i.e., greater than) compared to those of the Commercial Comparative Diapers 1 and 5.

For a comparison of average intake times of ADL adult incontinence product constructs in a no load saddle wicking test, using Code 1, 2, 3, or 4 composite fabric samples to replace the ADL of a Commercial Comparative adult incontinence product, the ADL adult incontinence product constructs made with Code 1, 2, 3, and 4 composite fabric samples showed improvement (i.e., lower intake times) in intake times 2 and 3 compared to the Commercial Comparative adult incontinence product.

For a comparison of wicking distances from insult point (front and back) and total wicking distance of ADL adult incontinence product constructs in a no load saddle wicking test, using Codes 1, 2, 3, or 4 composite fabric samples to replace the ADL of a Commercial Comparative adult incontinence product, the ADL adult incontinence product constructs made with Codes 1, 2, 3, and 4 composite fabric samples show improvement (i.e. greater wicking distances) in wicking distances when compared to the Commercial Comparative adult incontinence product.

In the present Example, for the majority of commercial baby diaper comparatives, Code 1 showed improvement in intake times and wicking distance for flat acquisition under load tests. For Commercial Comparative Diaper 1, the composite fabrics could assist absorbent cores with very high SAP content utilize more of the absorbent core than conventional ADLs. In both the flat acquisition under load and no load saddle wicking tests, ADL diaper constructs containing Codes 1, 2, 3, or 4 composite fabrics showed a significant increase in wicking distance.

### Example 7: Sequestration of TMA with Helix in Nonwovens

Helix™ in Nonwovens sheets were cut into 1 g pieces and compared with fiberized fluff, formed into pads, placed in sealed containers, and insulted with trimethylamine solution. The fiberized fluff is treated with a chemical to sequester trimethylamine.

Comparative fluff pulp sheets were cut into strips and then fiberized in a Kamas mill. The fluff pulp was then formed into 2-inch diameter pads with an average weight of 0.94±0.02 g. These pads were compressed in a Carver press to a pressure of 2000 psi.

Testing containers were constructed out of Kirkland 500 mL water bottles, which were selected due to their compressibility. 16 gauge needles were driven through plastic lids of the water bottles, glued in place, and sealed with silicone caulking. Rubber tubing was placed around the hilt of the needles to allow for an airtight seal between the hilt and measurement devices.

The compressed fluff rounds were introduced into the testing containers, insulted with 15 g of solution, sealed, and then the headspace above was tested for TMA after 2 hours. Referring to Table 11, TMA solutions were tested at a concentration of 0.053% by weight. Normal vaginal fluid not associated with bacterial vaginosis has trimethylamine levels 0.0005% by weight according to literature values.

TABLE 11
TMA solutions.

| | g DI | μL 25% | % by weight |
|---|---|---|---|
| | water | solution | TMA solutions |
| 25x literature | 300 | 639 | 0.053 |

The concentration of trimethylamine in the headspace of the containers was tested above both pulps two hours after insult. 105SE model Sensidyne® tubes were used. These tubes are labelled for use with ammonia, but are able to be used with trimethylamine, as well. The actual trimethylamine concentration is found by multiplying the Sensidyne® reading by a conversion factor of 0.5.

Three samples of each material were tested. The trimethylamine concentrations in headspaces above pads were compared for the Helix in Nonwovens composite and fluff pulp. Referring to Table 12, it was found that the Helix in Nonwovens composite decreased the headspace concentration of TMA more than the fluff pulp.

TABLE 12
TMA Headspace Test.

| | | Helix ™ | | |
|---|---|---|---|---|
| | Nonwoven | Air ®+ | | |
| | Basis | Basis | TMA | |
| | Weight | Weight | Concentration | TMA Present |
| Sample | (gsm) | (gsm) | (%) | (ppm) |
| NW/Helix ™ | 40 | 110 | 0.053 | 0.33 |
| Air ® 110 gsm | | | | |
| NW/Helix ™ | 40 | 80 | 0.053 | 0.33 |
| Air ®+ 80 gsm | | | | |
| NW/Helix ™ | 40 | 50 | 0.053 | 0.66 |
| Air ®+ 50 gsm | | | | |
| Bliss ™ | N/A | N/A | 0.053 | 17 |

### Example 8. Feminine Hygiene Product Evaluations

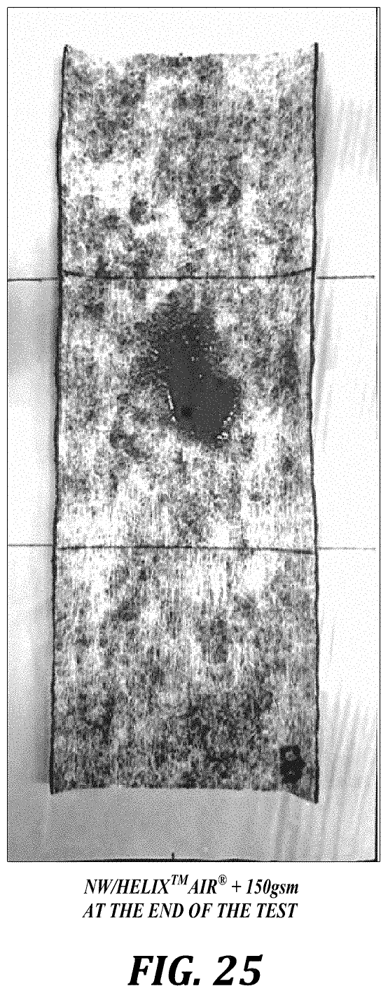

A NW/Helix™ Air®+ composite fabric having a basis weight of 150 g/m² was evaluated in a flat sheet configuration and served as an absorbent core for use in a sanitary pad. The nonwoven side faced the incoming liquid. Compared to 7 Commercial Comparative sanitary pads, referring to FIG. 25, the composite fabric had the most even fluid distribution. The basis weight of composite or the Helix™ Air®+ fraction did not appear to have an effect on distribution.

TABLE 13
Feminine hygiene product dimensions.

| | Absorbent Core |
|---|---|
| Product | (L × W) (cm × cm) |
| Commercial Comparative Sanitary Pad 1 | 19.5 × 7.2 |
| Commercial Comparative Sanitary Pad 2 | 20.5 × 6.2 |
| Commercial Comparative Sanitary Pad 3 | 22 × 7 |
| Commercial Comparative Sanitary Pad 4 | 19 × 7.4 |
| Commercial Comparative Sanitary Pad 5 | 22 × 6.6 |
| Commercial Comparative Sanitary Pad 6 | 18.3 × 6.7 |
| Commercial Comparative Sanitary Pad 7 | 17.4 × 7 |
| NW/Helix ™ Air ®+ composite fabric | 20 × 7 |

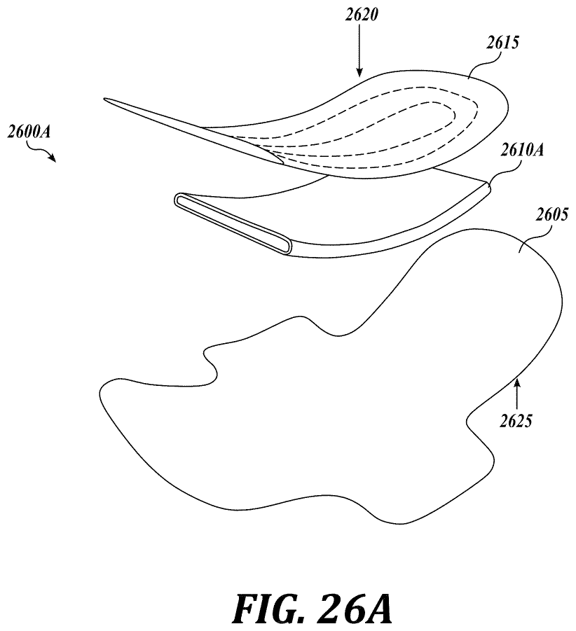

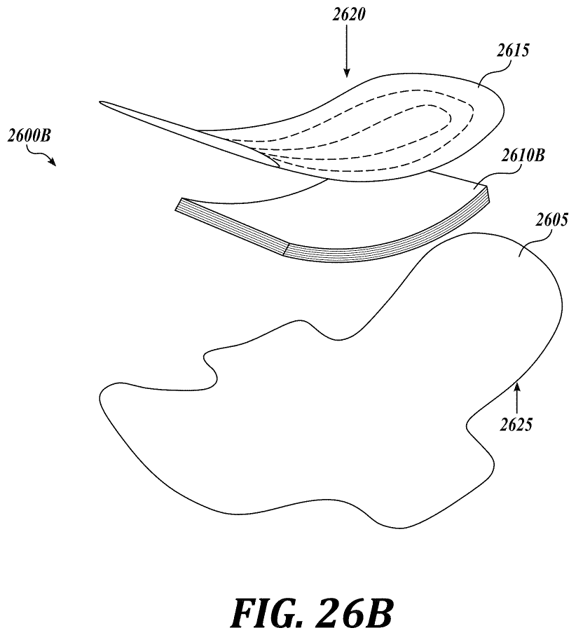

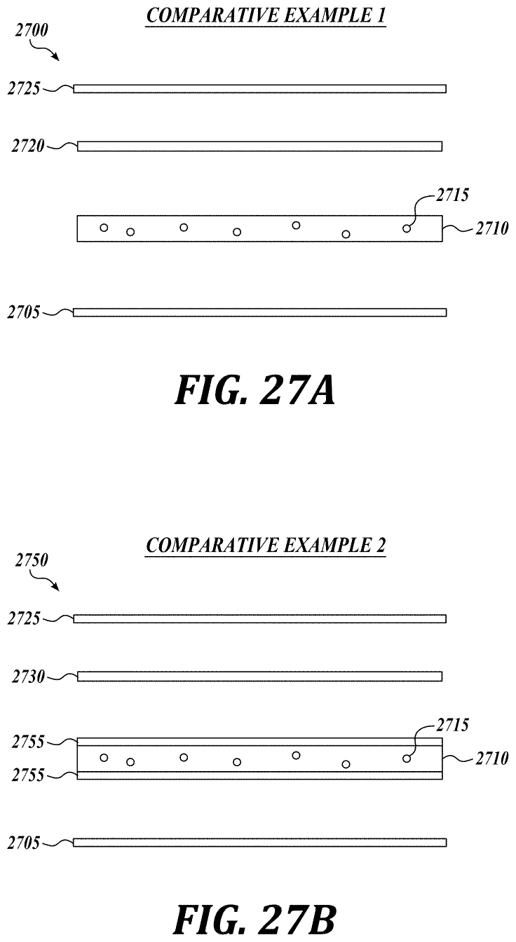

FIG. 26A is a schematic diagram illustrating an example feminine hygiene product 2600A including an absorbent composite 2610A, in accordance with embodiments of the present disclosure. Absorbent composite 2610A can be or include the composite fabrics formed by physically entangling a crosslinked cellulosic fiber layer and a nonwoven polymer layer, as described in more detail in reference to FIG. 1, as an example of composite fabric 110 of FIG. 1. Example feminine hygiene product 2600A also includes a liquid impermeable backsheet 2605 and topsheet 2615, as described in more detail in reference to FIG. 4. In contrast to conventional feminine hygiene products, described in reference to the comparative examples of FIGS. 27A and 27B, example feminine hygiene product 2600A can omit a dedicated acquisition-distribution layer (ADL), at least in part because composite fabric 2610A can perform the same or similar function with improved results, described below.

Example feminine hygiene product 2600A defines an inner surface 2620 and an outer surface 2625, relative to the orientation of donning and the expected direction of incidence of liquid insult. To that end, topsheet 2615 can be or include nonwoven material or another liquid-permeable material, such as a spunbond (SB) material or a through air bonded carded web (TABCW), or a perforated film. In some embodiments, topsheet 2615 can receive an embossed pattern or a pattern of perforations to facilitate or otherwise improve capillary action and/or wicking/distribution of liquid from the inner surface toward the absorbent composite 2610A. As such, the embossing and/or perforations can be propagated into and/or through the absorbent composite 2610A.

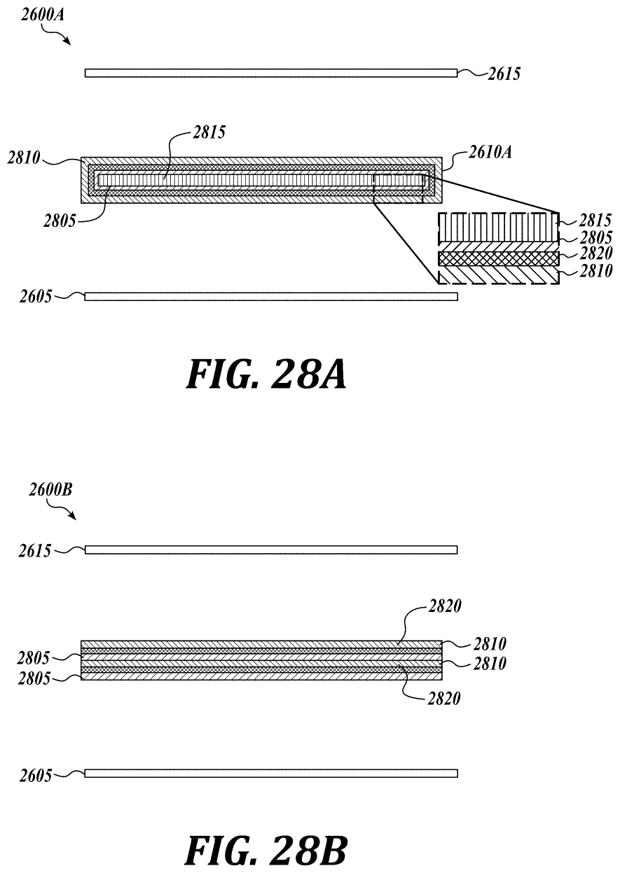

As illustrated in FIG. 26A, composite fabric 2610A can adopt a core-wrap configuration as described in more detail in reference to FIG. 28A. FIG. 26B, in contrast, illustrates a multilayer configuration where an absorbent composite 2610B includes one or more stacked layers of the physically entangled composite fabric described in reference to FIG. 1, in accordance with embodiments of the present disclosure. To that end, the stacked layers can be disposed relative to inner surface 2620 with crosslinked cellulose material physically contacting backsheet 2605.

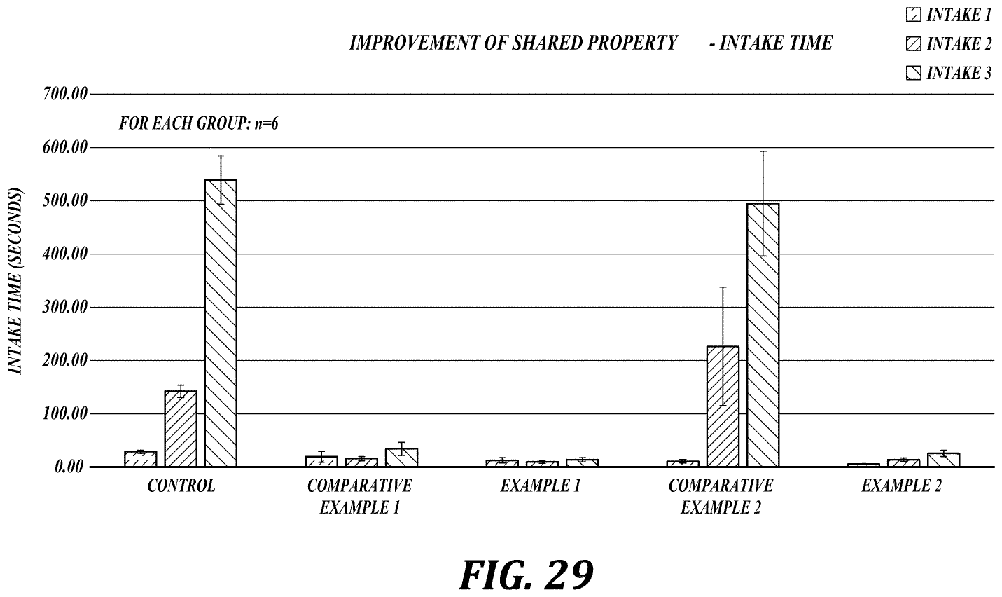

In reference to FIGS. 29-32 that present data generated by comparative testing of example feminine hygiene products 2600A and/or 2600B in accordance with embodiments of the present disclosure, FIGS. 27A-27B are schematic diagrams illustrating cross sections of comparative example 1 and comparative example 2, respectively. Comparative example 1 and comparative example 2 represent existing commercial feminine hygiene products considered to be market leaders in North American and Asian markets, respectively.

FIG. 27A illustrates an example commercial feminine hygiene product 2700 as available in the North American market. Example commercial feminine hygiene product 2700 includes a backsheet 2705, a cellulosic material layer 2710 that includes super-absorbent polymer 2715 dispersed in the cellulosic material layer 2710, an acquisition distribution layer 2720 formed of a spunlace material, and a liquid permeable topsheet 2725 formed of perforated film material. FIG. 27B illustrates an example commercial feminine hygiene product 2750 as available in the East Asian market. Example commercial feminine hygiene product 2750 includes a backsheet 2705, a cellulosic material layer 2710 that includes super-absorbent polymer 2715 dispersed in the cellulosic material layer 2710, an acquisition distribution layer 2730 formed of a through air bonded carded web (TABCW) material, and a liquid permeable topsheet 2725 formed of TABCW material. Additionally, example commercial feminine hygiene product 2750 includes two spunlace layers interposed between the cellulosic layer 2710 and the ADL 2730 and between the cellulosic layer 2710 and the backsheet 2705. Average physical characteristics of example commercial feminine hygiene products 2700 and 2750 are described in Table 14, in comparison to exemplary embodiments of the present disclosure. Data in Table 14 were tabulated from six examples of each product configuration.

TABLE 14
Average Material Data for Comparative Examples and Exemplary Embodiments

| Attribute (mean value) | Comparative | Comparative | | |
|---|---|---|---|---|
| *n = 6 | Example 1 | Example 2 | Example 1 | Example 2 |
| Absorbent Structure Mass (g) | 2.70 | 4.60 | 3.30 | 3.85 |
| Cellulose Mass (g) | 1.30 | 3.70 | 2.20 | 2.57 |
| Petrochemical Mass (g) | 1.40 | 0.9 | 1.10 | 1.28 |
| Absorbent Structure Caliper (mm) | 1.83 | 2.22 | 2.35 | 2.35 |

Advantageously, incorporating a smaller mass of petrochemical materials improves the overall sustainability of feminine hygiene products by reducing the use of petroleum that, on a global scale, represents an annual reduction of non-renewable resource usage on the order of thousands or millions of tons. Additionally, replacing petrochemical materials with cellulosic materials represents a replacement of a non-sustainable material with a renewable resource. In some embodiments, example feminine hygiene products 2600A and 2600 B include from about 0.5 g to about 5 g of cellulosic material, from about 1.0 g to about 5 g of cellulosic material, from about 1.5 g to about 5 g of cellulosic material, from about 2.0 g to about 5 g of cellulosic material, from about 2.5 g to about 5 g of cellulosic material, from about 3.0 g to about 5 g of cellulosic material, from about 3.5 g to about 5 g of cellulosic material, from about 4.0 g to about 5 g of cellulosic material, or from about 4.5 g to about 5 g of cellulosic material, including fractions and interpolations thereof. Understandably, while additional absorbent material will increase the overall volumetric capacity of example feminine hygiene product 2600A or 2600B, generally wearer experience is impaired with increasing product weight. As such, a lower mass can be desirable, balanced against the reduction of performance on acceptance and retention of liquid that results from reducing the quantity of absorbent material.

In some embodiments, example feminine hygiene products 2600A and 2600 B include from about 3.0 g to about 0.1 g of petrochemical material, from about 2.5 g to about 0.1 g of petrochemical material, from about 2.0 g to about 0.1 g of petrochemical material, from about 1.5 g to about 0.1 g of petrochemical material, from about 1.0 g to about 0.1 g of petrochemical material, and from about 0.5 g to about 0.1 g of petrochemical material, including fractions and interpolations thereof. As previously described, from a sustainability perspective, petrochemical materials can be sought to be excluded from example feminine hygiene product 2600A or 2600B to as great an extent as possible. Beyond a threshold value, however, structural and functional performance of products are impaired at the expense of wearer experience. For at least this reason, a petrochemical mass that is too low or zero can impair performance and result in larger sizes and weights, making feminine hygiene products feel bulky and large. Advantageously, Examples 1 and 2 exhibit a reduced mass of petrochemical materials relative to comparative example 1, without introducing the same additional amount of cellulosic material as in comparative example 2. In this way, the improved performance of example feminine hygiene products 2600A and 2600B can be seen to represent an improved approach for transitioning from petrochemical materials to cellulosic materials, in that performance is significantly improved with a smaller increase in product mass.

Advantageously, omitting dedicated ADL materials from example feminine hygiene products 2600A and 2600B, as described in more detail in reference to FIGS. 26A-26B and FIGS. 28A-28B can permit a larger portion of the absorbent structure to comprise cellulosic material without increasing characteristic dimensions, referred to as the “caliper,” at the expense of wearer experience. A thinner caliper, where caliper describes the thickness of components of the products, improves wearer experience by reducing the perception of wearing the product (e.g., during motion, when stretching, when seated, or when standing). Reducing the caliper by too great an extent, however, increases rewet and increases intake time by constraining fluid motion in the transverse direction between backsheet 2605 and topsheet 2615. In some embodiments, example feminine hygiene products 2600A and 2600 B have an absorbent structure caliper from about 3.0 mm to about 0.1 mm, from about 2.5 mm to about 0.1 mm, from about 2.0 mm to about 0.1 mm, from about 1.5 mm to about 0.1 mm, from about 1.0 mm to about 0.1 mm, and from about 0.5 mm to about 0.1 mm, including fractions and interpolations thereof. As such, experiments with example feminine hygiene product configurations resulted in performance data described in reference to FIGS. 29-32 for material and dimension data described in Table 14. As demonstrated in Table 14, exemplary embodiments of the present disclosure, as described in more detail in reference to FIGS. 26A-26B and 28A-28B, exhibit comparable dimensions and weight relative to comparative example 1 and comparative example 2. As such, data in Table 16, below, as well as the graphs presented in FIGS. 29-32, present direct comparisons between comparable products. In this way, the improvements to standard assay measures reported represent a significant and unexpected improvement to shared properties of intake time, rewet, wicking distance, or the like, in terms of both average performance and consistency between products.

To that end, FIG. 28A is a schematic diagram of a cross section of example feminine hygiene product 2600A illustrating material layers of absorbent composite material 2610A. As illustrated, in place of a dedicated ADL layer as is conventional in commercial products, example feminine hygiene product 2600A includes a core-wrap configuration of composite material formed from a nonwoven layer 2810 and a crosslinked cellulose layer 2805, wrapped around an absorbent core 2815. In some embodiments, the composite material is formed by physically entangling nonwoven layer 2810 and crosslinked cellulose layer 2805, which forms an interfacial region 2820 making the nonwoven layer 2810 and crosslinked cellulose layer 2805 mechanically inseparable in a dry state. In some embodiments, the composite material is at least partially wrapped around an absorbent core 2815. In some embodiments, the composite material is at least partially folded to form hollow core (e.g., omitting absorbent core 2815) or folded such that crosslinked cellulose layer 2805 contacts itself. Absorbent core 2815 can be or include a cellulosic fluff pulp material, a superabsorbent polymer, a synthetic absorbent material, or combinations thereof, as described in more detail in reference to FIGS. 8A-8C. The composite material can be wrapped completely around absorbent core 2815 or can be partially wrapped around absorbent core 2815, defining a gap that can be covered as described in more detail in reference to FIGS. 5B-7B. FIG. 28A is not drawn to scale, such that relative thicknesses of layers 2805 and 2810 and interfacial region 2820 are illustrated to facilitate visual interpretation, rather than to demonstrate exact or relative dimensions.

As described in more detail in reference to FIG. 26B, FIG. 28B is a schematic diagram illustrating a cross section of example feminine hygiene product 2600B, including absorbent composite material 2610B formed of multiple stacked layers of composite formed of crosslinked cellulose layer 2805 physically entangled with nonwoven layer 2810, with interfacial region 2820 formed therebetween. Example feminine hygiene product 2600B is illustrated including two layers of composite material. In some embodiments, example feminine hygiene product 2600B includes one layer, two layers, three layers, four layers, five layers, six layers, or more, of composite material. As described in reference to FIGS. 29-32, absorbent capacity increases with increasing layer number, making a higher number of layers beneficial up to a point that the caliper of the absorbent structure and the perceived size and weight of the example feminine hygiene product 2600B affects wearer comfort. To that end, the number of layers is selected to provide improved performance on the series of standardized assays described above without adding caliper or mass to a point that would impair wearer experience.

In some embodiments, example feminine hygiene product 2600B includes two layers of composite material, including two nonwoven layers 2810, two cellulose layers 2805, and two interfacial regions 2820, and outperforms both comparative example 1 and comparative example 2 on intake time, rewet, and wicking distance assays, as described in more detail in reference to FIGS. 29-32. With more layers of composite material, the likelihood of perceived weight and size causing discomfort increases. With fewer layers, the likelihood of impaired performance during liquid insult increases. As illustrated, nonwoven layer 2810 is oriented toward inner surface 2620 (e.g., facing topsheet 2615) and cellulose layer 2805 faces backsheet 2605. In this way, nonwoven layer 2810 can serve to distribute liquid through interfacial region 2820 to cellulose layer 2805 absorbing liquid wicked away from topsheet 2615 with improved performance over comparative examples. For layers nearer to backsheet 2605, liquid is received from layers nearer to topsheet 2615. In this way, while liquid insult can occur at a relatively concentrated region of an innermost layer of composite fabric (relative to inner surface 2620), the region of liquid transfer can increase with increasing layer number toward backsheet 2605, as absorbance in each respective layer includes a wicking component in a lateral direction and a longitudinal direction. In some embodiments, the orientations described above can be reversed, such that cellulose layers 2805 can be oriented toward inner surface 2620 and nonwoven layers 2810 oriented toward outer surface 2625.

In some embodiments, one or more layers of composite material are oriented in alternating or different configurations than those shown in FIG. 28B. For example, layers can be oriented such that at least one cellulose layer 2805 contacts another cellulose layer 2805, forming a dual-layer composite material with nonwoven layers 2810 facing both topsheet 2615 and backsheet 2605. In some embodiments, as when more than two layers of composite material are included, combinations of the two orientations described above can be included. For example, with three layers of composite material, a first layer and a second layer can be stacked with cellulose layer 2805 of the first layer contacting nonwoven layer 2810 of the second layer, with a third layer oriented such that the cellulose layers 2805 of the second and third layers contact each other.

Table 15 describes comparison data for performance of example feminine hygiene products 2600A and 2600B without topsheet 2615 on a series of assays described above in reference to absorbent article testing procedures. Table 15 is provided to demonstrate that both structures exhibit comparable performance for the suite of tests. Data in Table 15 are not representative of the performance of composite fabric in a complete feminine hygiene product, as such the data are not directly comparable to those of FIGS. 29-32. Instead, data in Table 15 demonstrate that the inclusion of composite fabric in one or more configurations can provide comparable performance of feminine hygiene products over a suite of assays representative of typical hygienic use.

TABLE 15
Performance Data of Example Absorbent Composites 2610A-B

| | Absorbent | Absorbent |
|---|---|---|
| Assay | Composite | Composite |
| N = 6, mean (std. dev) | 2610A | 2610B |
| Intake Time-First [Sec] | 9.5 (0.5) | 9.3 (1) |
| Intake Time-Second [Sec] | 24.7 (5) | 24.5 (2.5) |
| Intake Time-Third [Sec] | 54.2 (15) | 54.3 (11) |
| Rewet Mass [g] | 0.71 (0.18) | 0.72 (0.19) |
| Wicking Distance-Longitudinal [cm] | 9.8 (0.9) | 9.8 (0.5) |
| Wicking Distance-Lateral [cm] | 5.8 (0.8) | 6.0 (0.5) |

TABLE 16
Performance Data for Exemplary Embodiments and comparative examples

| | Intake | Intake | Intake | | Wicking | Wicking |
|---|---|---|---|---|---|---|
| Sample\Assay | Time- | Time- | Time- | | Distance- | Distance- |
| N = 6 mean | First | Second | Third | Rewet | Longitudinal | Lateral |
| (std. dev) | [Sec] | [Sec] | [Sec] | Mass [g] | [cm] | [cm] |
| Negative | 28.74 | 142.24 | 538.73 | 0.859 | 7.0 (0.0) | 3.3 (0.3) |
| Control | (2.92) | (11.62) | (45.38) | (0.085) | | |
| Comparative | 19.40 | 15.75 | 34.13 | 0.777 | 9.1 (0.2) | 5.4 (0.2) |
| Example 1 | (10.1) | (4.11) | (12.24) | (0.1) | | |
| Example 1 | 12.61 | 9.72 | 13.69 | 0.181 | 9.8 (0.0) | 6.0 (0.3) |
| | (4.94)) | (2.64) | (3.92) | (0.07) | | |
| Comparative | 10.74 | 226.48 | 494.58 | 1.160 | 8.1 (0.4) | 4.4 (0.2) |
| Example 2 | (2.80) | (111.24) | (98.45) | (0.115) | | |
| Example 2 | 5.88 | 13.79 | 25.77 | 0.583 | 9.2 (0.5) | 5.6 (0.4) |
| | (0.62) | (3.14) | (5.92) | (0.062) | | |

FIG. 29 is a graph of intake time data demonstrating improvement of a shared property between a negative control product without cellulosic materials, comparative example 1, comparative example 2, and example 1 and example 2 in accordance with embodiments of the present disclosure. Data for the graph in FIG. 29, reproduced in the first three columns of Table 16, include statistical measures developed from six replicate assays of each product, such that the bar height represents the average intake time and the error bars represent one standard deviation. As described above in reference to the absorbent article testing procedures, intake time assays include three sequential intake time tests to simulate repeated liquid insults on the same feminine hygiene product. In this way, the performance of the product under typical and extended wear conditions can be assessed. In FIG. 29, the control product is representative of commercially available light-weight feminine hygiene products that are constructed using synthetic materials, such as absorbent synthetic polymers, nonwoven acquisition distribution layers, or the like, without including cellulosic materials. Data for the control product is provided as an indirect comparison for the improvement to intake time performance of embodiments of the present disclosure. Comparative example 1 and comparative example 2, as described in Table 14, incorporate cellulosic materials as part of absorbent structures but do not include the composite fabric as described in more detail in reference to FIG. 1. As such, comparative example products 1 and 2 represent direct comparisons for a shared property to demonstrate the improved performance of embodiments of the present disclosure.

A lower intake time reflects faster distribution of liquid insults from the inner surface of a product (e.g., topsheet 2615) to absorbent materials that accept and retain liquid, keeping the wearer drier and more comfortable. Negative control product exhibits comparable performance to comparative example 2, with intake time increasing with each progressive insult, reaching a peak intake time of about 500 seconds for the third intake. In contrast, comparative example 1 and both examples 1 and 2 exhibit significantly reduced intake times relative to negative control. Relative to comparative example 2, example 2 exhibits significantly improved intake times for each intake, including approximately 20× improvement on second and third intakes. Furthermore, example 2 outperforms comparative example 1, despite incorporating additional internal structures and with different topsheet 2615 materials.

In this way, intake time for a first intake of example feminine hygiene products 2600A or 2600B can be less than or about 30 seconds, less than or about 29 seconds, less than or about 28 seconds, less than or about 27 seconds, less than or about 26 seconds, less than or about 25 seconds, less than or about 24 seconds, less than or about 23 seconds, less than or about 22 seconds, less than or about 21 seconds, less than or about 20 seconds, less than or about 19 seconds, less than or about 18 seconds, less than or about 17 seconds, less than or about 16 seconds, less than or about 15 seconds, less than or about 14 seconds, less than or about 13 seconds, less than or about 12 seconds, less than or about 11 seconds, less than or about 10 seconds, less than or about 9 seconds, less than or about 8 seconds, less than or about 7 seconds, less than or about 6 seconds, less than or about 5 seconds, less than or about 4 seconds, less than or about 3 seconds, less than or about 2 seconds, and less than or about 1 seconds, including fractions and interpolations thereof. Intake time for a second intake of example feminine hygiene products 2600A or 2600B can be less than or about 250 seconds, less than or about 240 seconds, less than or about 230 seconds, less than or about 220 seconds, less than or about 210 seconds, less than or about 200 seconds, less than or about 190 seconds, less than or about 180 seconds, less than or about 170 seconds, less than or about 160 seconds, less than or about 150 seconds, less than or about 140 seconds, less than or about 130 seconds, less than or about 120 seconds, less than or about 110 seconds, less than or about 100 seconds, less than or about 90 seconds, less than or about 80 seconds, less than or about 70 seconds, less than or about 60 seconds, less than or about 50 seconds, less than or about 40 seconds, less than or about 30 seconds, less than or about 20 seconds, less than or about 10 seconds, less than or about 9 seconds, less than or about 8 seconds, less than or about 7 seconds, less than or about 6 seconds, less than or about 5 seconds, less than or about 4 seconds, less than or about 3 seconds, less than or about 2 seconds, and less than or about 1 seconds, including fractions and interpolations thereof. Intake time for a third intake of example feminine hygiene products 2600A or 2600B can be less than or about 550 seconds, less than or about 525 seconds, less than or about 500 seconds, less than or about 475 seconds, less than or about 450 seconds, less than or about 425 seconds, less than or about 400 seconds, less than or about 375 seconds, less than or about 350 seconds, less than or about 325 seconds, less than or about 300 seconds, less than or about 275 seconds, less than or about 250 seconds, less than or about 225 seconds, less than or about 200 seconds, less than or about 175 seconds, less than or about 150 seconds, less than or about 125 seconds, less than or about 100 seconds, less than or about 75 seconds, less than or about 50 seconds, and less than or about 25 seconds, less than or about 10 seconds, less than or about 9 seconds, less than or about 8 seconds, less than or about 7 seconds, less than or about 6 seconds, less than or about 5 seconds, less than or about 4 seconds, less than or about 3 seconds, less than or about 2 seconds, and less than or about 1 seconds, including fractions and interpolations thereof.

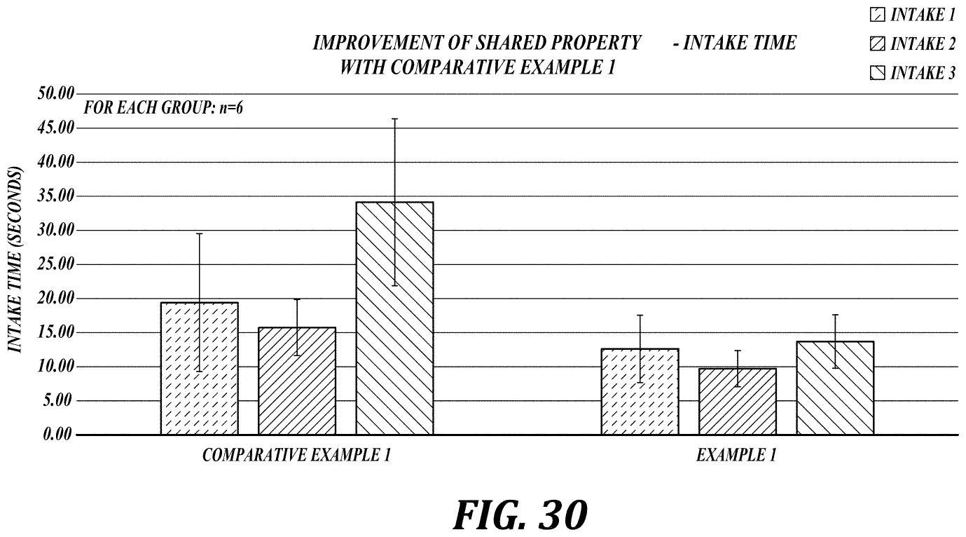

FIG. 30 is a graph of performance assay data of intake time for comparative example 1 and example 1, highlighting improved intake time performance of example feminine hygiene products 2600A and 2600B relative to comparative products from the North American market, in accordance with embodiments of the present disclosure. As shown, example 1 outperforms comparative example 1 on all three intake tests. Advantageously, example 1 exhibits a reduced intake time on all three intakes that is lower than the average intake time for first intake of comparative example 1. Data of FIG. 30 demonstrate that incorporating composite fabric 110 described in reference to FIG. 1 significantly improves performance on intake time, which directly reflects an improved wearer experience, while also omitting a dedicated ADL layer that is included in comparative example 1.

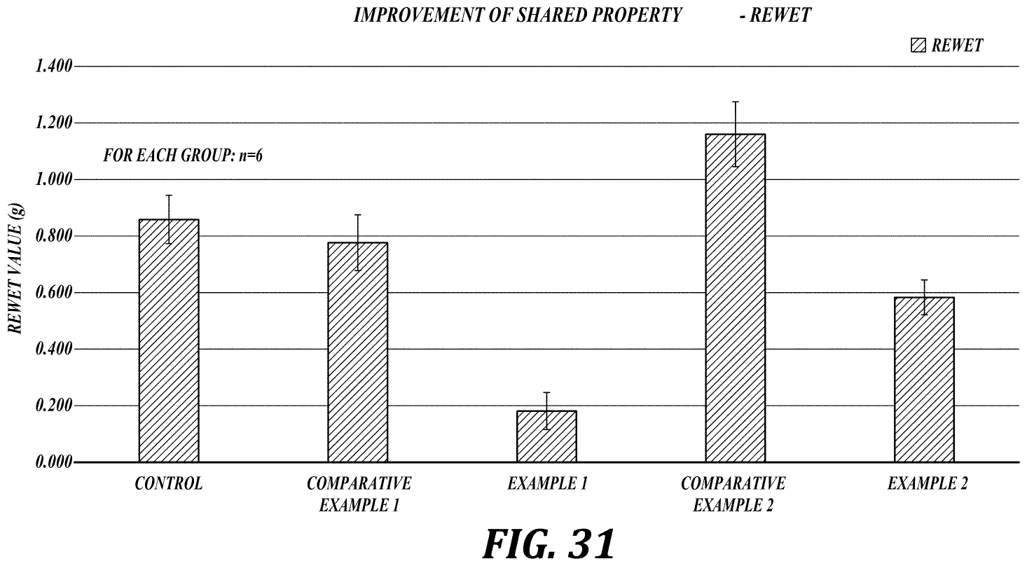

FIG. 31 is a graph of data from rewet mass assays for the same set of five products, in accordance with embodiments of the present disclosure. As previously described, rewet mass measures a quantity of liquid that is exuded from an absorbent article following application of a calibrated pressure. For that reason, a lower value of rewet mass reflects an improved retention of liquid under physically representative pressures, such as that of average bodyweight applied to a feminine hygiene product when seated. In this way, rewet mass for example feminine hygiene products 2600A or 2600B can be less than or about 1.5 grams, less than or about 1.4 grams, less than or about 1.3 grams, less than or about 1.2 grams, less than or about 1.1 grams, less than or about 1.0 grams, less than or about 0.9 grams, less than or about 0.8 grams, less than or about 0.7 grams, less than or about 0.6 grams, less than or about 0.5 grams, less than or about 0.4 grams, less than or about 0.3 grams, less than or about 0.2 grams, or less than or about 0.1 grams, including fractions and interpolations thereof. With improved rewet mass, examples 1 and 2 outperform both negative control and comparative examples 1 and 2, reflecting an improved wearer experience, while also omitting dedicated ADL layers that add material between absorbent components and topsheet 2615 that can serve to retain a portion of exuded liquid.

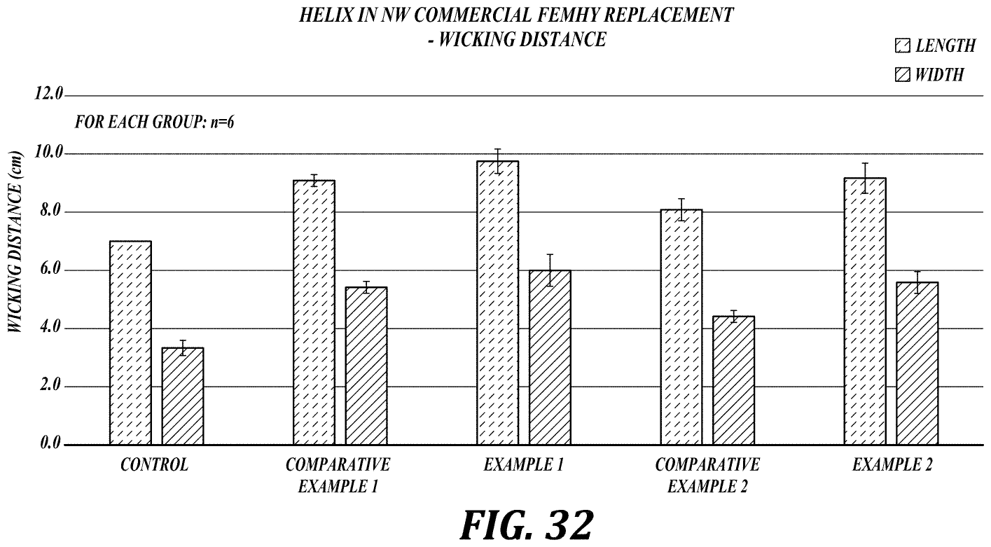

FIG. 32 is a graph of data from wicking distance assays for the same set of five products, in accordance with embodiments of the present disclosure. As previously described, wicking distance measures the performance of feminine hygiene products to accept and distribute a liquid insult during a prescribed period of time, in accordance with embodiments of the present disclosure. In this way, a longer wicking distance reflects improved performance. As examples 1 and 2 of example feminine hygiene product 2600A or 2600B omit a dedicated ADL, a person of ordinary skill would reasonably expect examples 1 and 2 to exhibit impaired performance relative to negative control and comparative examples 1 and 2, each of which include a dedicated ADL. Instead, FIG. 32 demonstrates that example 1 outperforms comparative example 1 in both longitudinal and lateral directions, reflecting improved wicking performance in both characteristic directions. Similarly, example 2 outperforms comparative example 2 in both longitudinal and lateral directions, reflecting improved wicking in both characteristic directions.

Improved performance on wicking distance assays reflects improved wearer experience at least in part due to improved distribution of a localized liquid insult through the feminine hygiene product to increase retention capacity over a time period that is characteristic of a period of wearing a feminine hygiene product. In some embodiments, longitudinal wicking distance for example feminine hygiene products 2600A or 2600B is greater than or about 0.5 cm, greater than or about 1.0 cm, greater than or about 1.5 cm, greater than or about 2.0 cm, greater than or about 2.5 cm, greater than or about 3.0 cm, greater than or about 3.5 cm, greater than or about 4.0 cm, greater than or about 4.5 cm, greater than or about 5.0 cm, greater than or about 5.5 cm, greater than or about 6.0 cm, greater than or about 6.5 cm, greater than or about 7.0 cm, greater than or about 7.5 cm, greater than or about 8.0 cm, greater than or about 8.5 cm, greater than or about 9.0 cm, greater than or about 9.5 cm, greater than or about 10.0 cm, greater than or about 10.5 cm, greater than or about 11.0 cm, greater than or about 11.5 cm, greater than or about 12.0 cm, greater than or about 12.5 cm, greater than or about 13.0 cm, greater than or about 13.5 cm, greater than or about 14.0 cm, greater than or about 14.5 cm, and greater than or about 15.0 cm, including fractions and interpolations thereof. In some embodiments, lateral wicking distance for example feminine hygiene products 2600A or 2600B is greater than or about 0.5 cm, greater than or about 1.0 cm, greater than or about 1.5 cm, greater than or about 2.0 cm, greater than or about 2.5 cm, greater than or about 3.0 cm, greater than or about 3.5 cm, greater than or about 4.0 cm, greater than or about 4.5 cm, greater than or about 5.0 cm, greater than or about 5.5 cm, greater than or about 6.0 cm, greater than or about 6.5 cm, greater than or about 7.0 cm, greater than or about 7.5 cm, greater than or about 8.0 cm, greater than or about 8.5 cm, greater than or about 9.0 cm, greater than or about 9.5 cm, greater than or about 10.0 cm, including fractions and interpolations thereof. Advantageously, data for example feminine hygiene products 2600A and 2600B, as illustrated in Table 16 and FIGS. 29-32, reveal improved performance in terms of average values and improved consistency as reflected by variability of measured values. For example, standard deviation values determined for each standard assay are lower for both Example 1 and Example 2 configurations, relative to the respective comparative example and negative control. In this way, embodiments of the present disclosure exhibit improved performance on average and more consistent performance, reflecting a significant improvement to wearer experience relative to conventional absorbent products currently available on the market.

By example and without limitation, embodiments are disclosed according to the following enumerated Paragraphs:

A1. A composite fabric, comprising:

a nonwoven layer comprising polymeric fibers and/or filaments;

a crosslinked cellulose layer comprising crosslinked cellulose fibers; wherein the crosslinked cellulose layer is positioned opposed to the nonwoven layer; and

an interfacial region between the nonwoven layer and the crosslinked cellulose layer, comprising physically entangled polymeric fibers and/or filaments from the nonwoven layer and crosslinked cellulose fibers from the crosslinked cellulose layer,

wherein the nonwoven layer and the crosslinked cellulose layer are mechanically inseparable in a dry state; and

wherein the composite fabric has a density of from 0.06 g/cm³ to 0.15 g/cm³ (e.g., 0.06 g/cm³, 0.12 g/cm³, 0.08 g/cm³, or 0.06-0.08 g/cm³).

A2. The composite fabric of Paragraph A1, wherein the nonwoven layer and the crosslinked cellulose layer overlap with one another and interpenetrate at the interfacial region.

A3. The composite fabric of Paragraph A1 or Paragraph A2, wherein the crosslinked cellulose layer and the nonwoven layer fully interpenetrate.

A4. The composite fabric of any one of the preceding Paragraphs, wherein the nonwoven layer has a first thickness, the crosslinked cellulose layer has a second thickness, and the interfacial region has a thickness less than or equal to the thickness of the first or the second thickness.

A5. The composite fiber of Paragraph A1, wherein the polymeric fibers and/or filaments comprises synthetic polymer fibers and/or filaments.

A6. The composite fabric of any one of the preceding Paragraphs, wherein the nonwoven layer comprises a bonded carded web fabric, a carded web, a spunbond fabric, a melt blown fabric, an unbonded synthetic fiber, or any combination thereof.

A7. The composite fabric of any one of the preceding Paragraphs, wherein the crosslinked cellulose fibers comprise polyacrylic acid crosslinked fibers.

A8. The composite fabric of any one of the preceding Paragraphs, wherein the crosslinked cellulose layer is air-laid or dry-laid onto the nonwoven layer.

A9. The composite fabric of any one of Paragraphs A1 to A7, wherein the crosslinked cellulose layer is wet-laid onto the nonwoven layer.

A10. The composite fabric of any one of Paragraphs A1 to A9, wherein the crosslinked cellulose fibers from the crosslinked cellulose layer are hydro-entangled into polymeric fibers and/or filaments from the nonwoven layer in the interfacial region.

A11. The composite fabric of any one of the preceding Paragraphs, wherein the nonwoven layer has a dry basis weight of 15 g/m² to 50 g/m² in the composite fabric.

A12. The composite fabric of any one of the preceding Paragraphs, wherein the crosslinked cellulose layer comprises a dry basis weight of 20 g/m² to 185 g/m² in the composite fabric.

A13. The composite fabric of any one of the preceding Paragraphs, wherein composite fabric is embossed, folded, pleated, and/or perforated, and wherein the folded or pleated composite fabric optionally comprises an absorbent material in a fold or a pleat.

A14. The composite fabric of any one of the preceding Paragraphs, wherein the composite fabric does not comprise latex, latex-bonded fibers, a hydroengorged layer, a pretreated nonwoven layer, lyocell, rayon, or any combination thereof.

A15. The composite fabric of any one of the preceding Paragraphs, consisting of the nonwoven layer and the crosslinked cellulose layer, and an interfacial region between the nonwoven layer and the crosslinked cellulose layer.

A16. The composite fabric of any one of the preceding Paragraphs, wherein the composite fabric neutralizes odor when subjected to biological fluids.

A17. An absorbent article, comprising the composite fabric of any one of the preceding Paragraphs.

A18. The absorbent article of Paragraph A17, wherein the article comprises a personal care absorbent product.

A19. The absorbent article of Paragraph A18, wherein the personal care absorbent product is selected from a diaper, an incontinence product, a feminine hygiene product, a wipe, a towel, and a tissue.

A20. The absorbent article of any one of Paragraphs A17 to A19, wherein the absorbent article comprises a fluid acquisition distribution layer comprising the composite fabric.

A21. The absorbent article of any one of Paragraphs A17 to A20, wherein the composite fabric is disposed over an absorbent material, wherein the crosslinked cellulose layer faces the surface of the absorbent material, and the absorbent material optionally comprises a superabsorbent polymer.

A22. The absorbent article of any one of Paragraphs A17 to A19, further comprising an absorbent core.

A23. The absorbent article of Paragraph A22, wherein the absorbent core comprises a first layer of composite fabric overlying an absorbent material and a second layer of composite fabric underlying the absorbent material, wherein the absorbent material optionally comprises a superabsorbent polymer.

A24. The absorbent article of Paragraph A22, wherein the absorbent core comprises the composite fabric enveloping an absorbent material, wherein the absorbent material optionally comprises a superabsorbent polymer.

A25. The absorbent article of Paragraph A24, wherein the composite fabric fully envelops the absorbent material, wherein the absorbent material optionally comprises a superabsorbent polymer.

A26. The absorbent article of Paragraph A24 or Paragraph A25, wherein the crosslinked cellulose layer contacts the surface of the absorbent material.

A27. The absorbent article of Paragraphs A17 to A20 and A22 to A25, wherein the absorbent article comprises an absorbent material, wherein either the nonwoven layer or the crosslinked cellulose layer contacts the surface of the absorbent material, when the composite fabric is folded or pleated.

A28. The absorbent article of any one of Paragraphs A18 to A27, wherein the absorbent article is a diaper or an incontinence product.

A29. The absorbent article of any one of Paragraphs A20, A21, A26 and A27, wherein the absorbent article has an intake time decrease of at least 23% from a first fluid exposure to a second subsequent fluid exposure in a flat acquisition under load test, when the absorbent article comprises a fluid acquisition distribution layer comprising the composite fabric.

A30. The absorbent article of any one of Paragraphs A24 to A28, wherein the absorbent article has an intake time decrease of at least 25% from a first fluid exposure to a second subsequent fluid exposure in a flat acquisition under load test, when the absorbent article comprises the composite fabric enveloping the absorbent core.

A31. The absorbent article of any one of Paragraphs A20, A21, and A28, wherein the absorbent article has an intake time decrease of at least 8% from a second fluid exposure to a third subsequent fluid exposure in a flat acquisition under load test, when the absorbent article comprises a fluid acquisition distribution layer comprising the composite fabric.

A32. The absorbent article of any one of Paragraphs A24 to A28, wherein the absorbent article has an intake time decrease of at least 12% from a second fluid exposure to a third subsequent fluid exposure in a flat acquisition under load test, when the absorbent article comprises the composite fabric enveloping the absorbent material.

A33. The absorbent article of any one of Paragraphs A20, A21, and A28, wherein the absorbent article has a wicking distance percentage of at least 60% after a third fluid exposure in a no load saddle wicking test when the absorbent article comprises a fluid acquisition distribution layer comprising the composite fabric.

A34. The absorbent article of any one of Paragraphs A24 to A28, wherein the absorbent article has a wicking distance percentage of at least 60% after a third fluid exposure in a no load saddle wicking test when the absorbent article comprises the composite fabric enveloping the absorbent material.

A35. The absorbent article of any one of Paragraphs A17 to A21 and A28, wherein the composite fabric comprises the nonwoven layer at a dry basis weight of 20 g/m² to 50 g/m² (e.g., 30 g/m² to 40 g/m²) and the crosslinked cellulose layer at a dry basis weight of 70 g/m² to 120 g/m² (e.g., 80 g/m² to 110 g/m²).

A36. The absorbent article of any one of Paragraphs A17 to A19, and A22 to A28, wherein the composite fabric comprises the nonwoven layer at a dry basis weight of 20 g/m² to 50 g/m² (e.g., 30 g/m² to 40 g/m²) and the crosslinked cellulose layer at a dry basis weight of 40 g/m² to less than 70 g/m² (e.g., 40 g/m² to 60 g/m², or 50 g/m²).

A37. The absorbent article of Paragraph A35, wherein the absorbent article has a wicking distance percentage of at least 60% after a third fluid exposure in a no load saddle wicking test when the absorbent article comprises a fluid acquisition distribution layer comprising the composite fabric.

A38. The absorbent article of Paragraph A36, wherein the absorbent article has a wicking distance percentage of at least 60% after a third fluid exposure in a no load saddle wicking test when the absorbent article comprises the composite fabric enveloping the absorbent material.

A39. An absorbent article, comprising:

a liquid-impermeable backsheet defining an inner surface and an outer surface;

an absorbent core, disposed on the inner surface of the backsheet, wherein the absorbent core comprises:
an absorbent material defining an upper surface and a lower surface of the absorbent core; and
a composite fabric surrounding at least a portion of the upper surface and the lower surface, comprising:
   a nonwoven layer comprising polymeric fibers and/or filaments;
   a crosslinked cellulose layer comprising crosslinked cellulose fibers, wherein the crosslinked cellulose layer is positioned opposed to the nonwoven layer; and
   an interfacial region between the nonwoven layer and the crosslinked cellulose layer, comprising physically entangled polymeric fibers and/or filaments from the nonwoven layer and crosslinked cellulose fibers from the crosslinked cellulose layer,
   wherein the nonwoven layer and the crosslinked cellulose layer are mechanically inseparable in a dry state; and

a topsheet overlying the upper surface of the absorbent core and contacting the inner surface of the backsheet.

A40. The absorbent article of Paragraph A39, wherein the composite fabric fully surrounds the upper surface and the lower surface of the absorbent core.

A41. The absorbent article of Paragraph A39, wherein the composite fabric overlaps on the upper surface or the lower surface of the absorbent core by at least a portion of a width of the composite fabric.

A42. The absorbent article of Paragraph A39, wherein the composite fabric defines a gap on the upper surface or the lower surface of the absorbent core, the absorbent core further comprising a cover layer disposed over the gap.

A43. The absorbent article of Paragraph A42, wherein the cover layer overlies at least a portion of the composite fabric, the composite fabric being disposed between at least a portion of the cover layer and the absorbent material.

A44. The absorbent article of Paragraph A42, wherein the cover layer underlies the composite fabric, and at least a portion of the cover layer is disposed between the composite fabric and the absorbent material.

A45. The absorbent article of any one of Paragraphs A42 to A44, wherein the cover layer is formed of the composite fabric.

A46. The absorbent article of any one of Paragraphs A42 to A45, wherein the cover layer comprises a spunbond meltblown spunbond (SMS) material.

A47. The absorbent article of any one of Paragraphs A42 to A45, wherein the cover layer comprises a spunbond (SB) material.

A48. The absorbent article of any one of Paragraphs A39 to A47, wherein the absorbent material comprises an absorbent synthetic polymer and a high-loft through air bonded carded web (TABCW).

A49. The absorbent article of any one of Paragraphs A39 to A47, wherein the absorbent material comprises an absorbent synthetic polymer (e.g., SAP), a fluff pulp, or any combination thereof.

A50. The absorbent article of Paragraph A49, wherein the absorbent material comprises from 30% to 90% by weight of the absorbent synthetic polymer and from 10% to 70% by weight of the fluff.

A51. The absorbent article of any one of Paragraphs A39 to A50, wherein the polymeric fibers and/or filaments of the nonwoven layer of the composite fabric comprises synthetic polymer fibers and/or filaments.

A52. The absorbent article of any one of Paragraphs A39 to A51, wherein the nonwoven layer and the crosslinked cellulose layer of the composite fabric overlap with one another and interpenetrate at the interfacial region.

A53. The absorbent article of any one of Paragraphs A39 to A52, wherein the crosslinked cellulose layer and the nonwoven layer of the composite fabric fully interpenetrate.

A54. The absorbent article of any one of Paragraphs A39 to A52, wherein the nonwoven layer has a first thickness, the crosslinked cellulose layer has a second thickness, and interfacial region comprises a thickness less than or equal to the thickness of the first or the second thickness.

A55. The absorbent article of any one of Paragraphs A39 to A54, wherein the nonwoven layer comprises a bonded carded web fabric, a carded web, a spunbond fabric, a melt blown fabric, or any combination thereof.

A56. The absorbent article of any one of Paragraphs A39 to A55, wherein the crosslinked cellulose fibers comprise polyacrylic acid crosslinked fibers.

A57. The absorbent article of any one of Paragraphs A39 to A56, wherein the crosslinked cellulose fibers from the crosslinked cellulose layer are hydro-entangled into polymeric fibers and/or filaments from the nonwoven layer in the interfacial region.

A58. The absorbent article of any one of Paragraphs A39 to A57, wherein the nonwoven layer has a dry basis weight of 15 g/m² to 50 g/m² in the composite fabric.

A59. The absorbent article of any one of Paragraphs A39 to A58, wherein the crosslinked cellulose layer comprises a dry basis weight of 20 g/m² to 185 g/m² in the composite fabric.

A60. The absorbent article of any one of Paragraphs A39 to A59, wherein the composite fabric does not comprise latex, latex-bonded fibers, a hydroengorged layer, a pretreated nonwoven layer, lyocell, rayon, or any combination thereof.

A61. The absorbent article of any one of Paragraphs A39 to A60, wherein the article comprises a personal care absorbent product.

A62. The absorbent article of Paragraph A61, wherein the personal care absorbent product is selected from a diaper, an incontinence product, and a feminine hygiene product.

A63. The absorbent article of any one of Paragraphs A39 to A62, wherein the composite fabric fully envelops an absorbent material, wherein the absorbent material optionally comprises a superabsorbent polymer.

A64. The absorbent article of any one of Paragraphs A39 to A63, wherein the crosslinked cellulose layer contacts the surface of the absorbent material.

A65. The absorbent article of any one of Paragraphs A39 to A64, wherein the absorbent article has an intake time decrease of at least 25% from a first fluid exposure to a second subsequent fluid exposure in a flat acquisition under load test.

A66. The absorbent article of any one of Paragraphs A39 to A65, wherein the absorbent article has an intake time decrease of at least 12% from a second fluid exposure to a third subsequent fluid exposure in a flat acquisition under load test.

A67. The absorbent article of any one of Paragraphs A39 to A66, wherein the absorbent article has a wicking distance percentage of at least 60% after a third fluid exposure in a no load saddle wicking test when the absorbent article comprises the composite fabric enveloping the absorbent material.

A68. The absorbent article of any one of Paragraphs A39 to A67, wherein the composite fabric comprises the nonwoven layer at a dry basis weight of 20 g/m² to 50 g/m² (e.g., 30 g/m² to 40 g/m²) and the crosslinked cellulose layer at a dry basis weight of 40 g/m² to less than 70 g/m² (e.g., 40 g/m² to 60 g/m², or 50 g/m²).

A69. A feminine hygiene product, comprising:

a composite fabric comprising:
a nonwoven layer comprising polymeric fibers and/or filaments;
a crosslinked cellulose layer comprising crosslinked cellulose fibers, wherein the crosslinked cellulose layer is positioned opposed to the nonwoven layer; and
an interfacial region between the nonwoven layer and the crosslinked cellulose layer, comprising physically entangled polymeric fibers and/or filaments from the nonwoven layer and crosslinked cellulose fibers from the crosslinked cellulose layer,
wherein the nonwoven layer and the crosslinked cellulose layer are mechanically inseparable in a dry state.

A70. The feminine hygiene product of Paragraph A69, further comprising an absorbent core comprising an absorbent material.

A71. The feminine hygiene product of Paragraph A69 or Paragraph A70, wherein when subjected to a fluid insult, the composite fabric distributes the fluid to a front portion, a middle portion, and a back portion of the feminine hygiene product.

A72. The feminine hygiene product of Paragraph A71, wherein the front portion, middle portion, and back portion each comprises an amount of fluid within 20 wt % to 45 wt % of each portion.

A73. The feminine hygiene product of any one of Paragraphs A70 to A72, wherein the composite fabric is disposed over the absorbent core.

A74. The feminine hygiene product of any one of Paragraphs A70 to A72, wherein the composite fabric envelops at least a portion of the absorbent material.

A75. A method of making a composite fabric of any one of Paragraphs A1 to A15, comprising:

supplying polymeric fibers and/or filaments;

supplying crosslinked cellulose fibers;

air-laying or wet-laying the crosslinked cellulose fibers to provide a crosslinked cellulose layer on a nonwoven layer of polymeric fibers and/or filaments, wherein the crosslinked cellulose layer is positioned opposed to the nonwoven layer; and physically entangling the polymeric fibers and/or filaments from the nonwoven layer and the crosslinked cellulose fibers from the crosslinked cellulose layer to provide the composite fabric, wherein the composite fabric comprises an interfacial region between the nonwoven layer and the crosslinked cellulose layer, wherein the nonwoven layer and the crosslinked cellulose layer are mechanically inseparable in a dry state.

A76. The method of Paragraph A75, wherein physically entangling the polymeric fibers and/or filaments from the nonwoven layer and the crosslinked cellulose fibers from the crosslinked cellulose layer comprises hydro-entangling the crosslinked cellulose fibers into the polymeric fibers and/or filaments.

A77. The method of Paragraph A75 or Paragraph A76, wherein the polymeric fibers and/or filaments is in the form of a bonded carded web fabric, a carded web, a spunbond fabric, a melt blown fabric, an unbonded synthetic fiber, or any combination thereof.

A78. The method of any one of Paragraphs A75 to A77, wherein the polymeric fibers are synthetic.

A79. The method of any one of Paragraphs A75 to A78, wherein the nonwoven layer is a top layer, and the crosslinked cellulose layer is a bottom layer.

A80. The method of any one of Paragraphs A75 to A78, wherein the nonwoven layer is a bottom layer, and the crosslinked cellulose layer is a top layer.

A81. The method of any one of Paragraphs A75 to A80, wherein the crosslinked cellulose layer is pre-formed prior to entangling with the nonwoven layer, and/or the nonwoven layer is pre-formed prior to entangling with the crosslinked cellulose layer.

A82. The method of any one of Paragraphs A75 to A80, wherein the crosslinked cellulose layer is not pre-formed prior to entangling with the nonwoven layer, and/or the nonwoven layer is not pre-formed prior to entangling with the crosslinked cellulose layer.

A83. A feminine hygiene product, comprising:

a composite fabric comprising:
a nonwoven layer comprising polymeric fibers and/or filaments;
a crosslinked cellulose layer comprising crosslinked cellulose fibers, wherein the crosslinked cellulose layer is positioned opposed to the nonwoven layer; and
an interfacial region between the nonwoven layer and the crosslinked cellulose layer, comprising physically entangled polymeric fibers and/or filaments from the nonwoven layer and crosslinked cellulose fibers from the crosslinked cellulose layer,

wherein the nonwoven layer and the crosslinked cellulose layer are mechanically inseparable in a dry state.

A84. The feminine hygiene product of any one of Paragraph A83, further comprising:

an absorbent core including a superabsorbent polymer,

wherein the composite fabric is wrapped at least partially around the absorbent core with the crosslinked cellulose layer physically contacting the absorbent core.

A85. The feminine hygiene product of any one of Paragraphs A83 to A84, wherein the nonwoven layer is a first nonwoven layer, the crosslinked cellulose layer is a first crosslinked cellulose layer, and the interfacial region is a first interfacial region, the composite fabric further comprising:

a second nonwoven layer;

a second crosslinked cellulose layer; and

a second interfacial region between the second nonwoven layer and the second crosslinked cellulose layer, comprising physically entangled polymeric fibers and/or filaments from the second nonwoven layer and crosslinked cellulose fibers from the second crosslinked cellulose layer,

wherein the second nonwoven layer and the second crosslinked cellulose layer are mechanically inseparable in a dry state, and wherein the second crosslinked cellulose layer is mechanically coupled with the first nonwoven layer.

A86. The feminine hygiene product of any one of Paragraphs A83 to A85, further comprising an average intake time on a first intake of less than or equal to about 15 seconds, as measured by a standardized intake time method.

A87. The feminine hygiene product of any one of Paragraphs A83 to A86, further comprising an average intake time on a second intake of less than or equal to about 15 seconds, as measured by a standardized intake time method.

A88. The feminine hygiene product of any one of Paragraphs A83 to A87, further comprising an average intake time on a second intake of less than or equal to about 10 seconds, as measured by the standardized intake time method.

A89. The feminine hygiene product of any one of Paragraphs A83 to A88, further comprising an average intake time on a third intake of less than or equal to about 30 seconds, as measured by a standardized intake time method.

A90. The feminine hygiene product of any one of Paragraphs A83 to A89, further comprising an average intake time on a third intake of less than or equal to about 15 seconds, as measured by the standardized intake time method.

A91. The feminine hygiene product of any one of Paragraphs A83 to A90, further comprising an average rewet mass of less than or equal to about 0.75 grams, as measured by a standardized rewet performance method.

A92. The feminine hygiene product of any one of Paragraphs A83 to A91, further comprising an average rewet mass of less than or equal to about 0.20 grams, as measured by the standardized rewet performance method.

A93. The feminine hygiene product of any one of Paragraphs A83 to A92, further comprising an average wicking distance in a longitudinal direction of greater than or equal to about 9.2 cm, as measured by a standardized liquid distribution method.

A94. The feminine hygiene product of any one of Paragraphs A83 to A93, further comprising an average wicking distance in a longitudinal direction of greater than or equal to about 9.8 cm, as measured by the standardized liquid distribution method.

A95. The feminine hygiene product of any one of Paragraphs A83 to A94, further comprising:

a topsheet comprising a nonwoven material, the topsheet mechanically coupled with the composite fabric and defining an inner surface of the feminine hygiene product; and

a liquid impermeable backsheet mechanically coupled with the composite fabric and defining an outer surface of the feminine hygiene product.

A96. The feminine hygiene product of any one of Paragraphs A83 to A95, wherein the topsheet comprises a perforated film material or a through air bonded carded web (TABCW).

A97. The feminine hygiene product of any one of Paragraphs A83 to A96, further comprising:

a first spunlace layer interposed between the topsheet and the composite fabric; and

a second spunlace layer interposed between the liquid impermeable backsheet and the composite fabric.

A98. The feminine hygiene product of any one of Paragraphs A83 to A97, further comprising an average intake time on a second intake of less than or equal to about 225 seconds, as measured by a standardized intake time method.

A99. The feminine hygiene product of any one of Paragraphs A83 to A98, further comprising an average intake time on a third intake of less than or equal to about 490 seconds, as measured by a standardized intake time method.

A100. The feminine hygiene product of any one of Paragraphs A83 to A99, further comprising an average rewet mass of less than or equal to about 1.15 grams, as measured by a standardized rewet performance method.

A101. The feminine hygiene product of any one of Paragraphs A83 to A100, further comprising an average wicking distance in a longitudinal direction of greater than or equal to about 8.2 cm, as measured by a standardized rewet performance method.

A102. The feminine hygiene product of any one of Paragraphs A83 to A101, further comprising an average wicking distance in a lateral direction of greater than or equal to about 4.5 cm, as measured by a standardized liquid distribution method.

A103. The feminine hygiene product of any one of Paragraphs A83 to A102, further comprising a total mass of composite fabric of less than or equal to about 4.00 g.

A104. The feminine hygiene product of any one of Paragraphs A83 to A103, further comprising a total mass of cellulosic material of greater than or equal to about 1.50 g.

A105. The feminine hygiene product of any one of Paragraphs A83 to A104, further comprising a total mass of petrochemical material of less than or equal to about 1.30 g.

While illustrative embodiments have been illustrated and described, it will be appreciated that various changes can be made therein without departing from the spirit and scope of the disclosure.

The embodiments of the invention in which an exclusive property or privilege is claimed are defined as follows:

1. A feminine hygiene product, comprising:
a composite fabric comprising:
a nonwoven layer comprising polymeric fibers and/or filaments;
a crosslinked cellulose layer comprising crosslinked cellulose fibers, wherein the crosslinked cellulose layer is positioned opposed to the nonwoven layer; and
an interfacial region between the nonwoven layer and the crosslinked cellulose layer, comprising physically entangled polymeric fibers and/or filaments from the nonwoven layer and crosslinked cellulose fibers from the crosslinked cellulose layer,
wherein the nonwoven layer and the crosslinked cellulose layer are mechanically inseparable in a dry state.

2. The feminine hygiene product of claim 1, further comprising:
an absorbent core including a superabsorbent polymer,
wherein the composite fabric is wrapped at least partially around the absorbent core with the crosslinked cellulose layer physically contacting the absorbent core.

3. The feminine hygiene product of claim 1, wherein the nonwoven layer is a first nonwoven layer, the crosslinked cellulose layer is a first crosslinked cellulose layer, and the interfacial region is a first interfacial region, the composite fabric further comprising:
a second nonwoven layer;
a second crosslinked cellulose layer; and
a second interfacial region between the second nonwoven layer and the second crosslinked cellulose layer, comprising physically entangled polymeric fibers and/or filaments from the second nonwoven layer and crosslinked cellulose fibers from the second crosslinked cellulose layer,
wherein the second nonwoven layer and the second crosslinked cellulose layer are mechanically inseparable in a dry state, and wherein the second crosslinked cellulose layer is mechanically coupled with the first nonwoven layer.

4. The feminine hygiene product of claim 1, further comprising an average intake time on a first intake of less than or equal to about 15 seconds, as measured by a standardized intake time method.

5. The feminine hygiene product of claim 1, further comprising an average intake time on a second intake of less than or equal to about 15 seconds, as measured by a standardized intake time method.

6. The feminine hygiene product of claim 4, further comprising an average intake time on a second intake of less than or equal to about 10 seconds, as measured by the standardized intake time method.

7. The feminine hygiene product of claim 1, further comprising an average intake time on a third intake of less than or equal to about 30 seconds, as measured by a standardized intake time method.

8. The feminine hygiene product of claim 7, further comprising an average intake time on a third intake of less than or equal to about 15 seconds, as measured by the standardized intake time method.

9. The feminine hygiene product of claim 1, further comprising an average rewet mass of less than or equal to about 0.75 grams, as measured by a standardized rewet performance method.

10. The feminine hygiene product of claim 9, further comprising an average rewet mass of less than or equal to about 0.20 grams, as measured by the standardized rewet performance method.

11. The feminine hygiene product of claim 1, further comprising an average wicking distance in a longitudinal direction of greater than or equal to about 9.2 cm, as measured by a standardized liquid distribution method.

12. The feminine hygiene product of claim 11, further comprising an average wicking distance in a longitudinal direction of greater than or equal to about 9.8 cm, as measured by the standardized liquid distribution method.

13. The feminine hygiene product of claim 1, further comprising an average wicking distance in a lateral direction of greater than or equal to about 5.5 cm, as measured by a standardized liquid distribution method.

14. The feminine hygiene product of claim 1, further comprising:
a topsheet comprising a nonwoven material, the topsheet mechanically coupled with the composite fabric and defining an inner surface of the feminine hygiene product; and
a liquid impermeable backsheet mechanically coupled with the composite fabric and defining an outer surface of the feminine hygiene product.

15. The absorbent article of claim 14, wherein the topsheet comprises a perforated film material or a through air bonded carded web (TABCW).

16. The feminine hygiene product of claim 14, further comprising:
a first spunlace layer interposed between the topsheet and the composite fabric; and
a second spunlace layer interposed between the liquid impermeable backsheet and the composite fabric.

17. The feminine hygiene product of claim 16, further comprising an average intake time on a second intake of less than or equal to about 225 seconds, as measured by a standardized intake time method.

18. The feminine hygiene product of claim 16, further comprising an average intake time on a third intake of less than or equal to about 490 seconds, as measured by a standardized intake time method.

19. The feminine hygiene product of claim 16, further comprising an average rewet mass of less than or equal to about 1.15 grams, as measured by a standardized rewet performance method.

20. The feminine hygiene product of claim 16, further comprising an average wicking distance in a longitudinal direction of greater than or equal to about 8.2 cm, as measured by a standardized rewet performance method.

21. The feminine hygiene product of claim 16, further comprising an average wicking distance in a lateral direction of greater than or equal to about 4.5 cm, as measured by a standardized liquid distribution method.

22. The feminine hygiene product of claim 1, further comprising a total mass of composite fabric of less than or equal to about 4.00 g.

23. The feminine hygiene product of claim 1, further comprising a total mass of cellulosic material of greater than or equal to about 1.50 g.

24. The feminine hygiene product of claim 1, further comprising a total mass of petrochemical material of less than or equal to about 1.30 g.
